(19) United States
(12) Patent Application Publication
(10) Pub. No.: US 2009/0124610 A1
(43) Pub. Date: May 14, 2009

(54) PHARMACEUTICAL COMPOUNDS

(76) Inventors: Gordon Saxty, Cambridge (GB); Marinus Leendert Verdonk, Burwell (GB); John Caldwell, Sutton (GB); Ian Collins, Redhill (GB); Kwai-Ming Cheung, Staines (GB); Tatiana Faria Da Fonseca McHardy, Sunbury-on-Thames (GB)

Correspondence Address: HESLIN ROTHENBERG FARLEY & MESITI PC, 5 COLUMBIA CIRCLE, ALBANY, NY, 12203, US

(21) Appl. No.: 12/298,384

(22) PCT Filed: Apr. 25, 2007
(86) PCT No.: PCT/GB2007/001517

§ 371 Date: Oct. 24, 2008

Related U.S. Application Data
(60) Provisional application No. 60/745,555, filed on Apr. 25, 2006.

(30) Foreign Application Priority Data
Apr. 25, 2006 (GB) 0608162.4

(51) Int. Cl. A61K 31/5383; C07D 495/04; A61K 31/519; A61K 31/496; C07D 211/56; A61K 31/44; C07D 409/14; A61K 31/445; C07D 211/06; C07D 401/14; A61K 31/437; C07D 413/14

(52) U.S. Cl. 514/230.5; 544/278; 514/260.1; 514/252.16; 546/223; 514/352; 514/354; 546/226; 546/113; 514/300; 544/105

(57) ABSTRACT

Compounds of the formula (I), and salts, solvates, tautomers and N-oxide thereof, wherein TG is selected from groups (1) and (2): wherein the asterisk (*) represents the point of attachment of the group E to the group X; R^{1a} is an optionally substituted aryl or heteroaryl group; R^{1b} is hydrogen or a group R1a; X is an optionally substituted bicyclic heterocyclic group having 8 to 12 ring members of which up to 5 are heteroatoms selected from O, N and S; and A, E, R², R³, R⁴, Q¹ and Q² are as defined in the claims; provided that when E is aryl or heteroaryl, then Q² is other than a bond; and further provided that the moiety (a) is other than a group (BG1) or (BG2); wherein (BG1) and (BG2) are each optionally substituted; T is N or CR^{Z}; J¹-J² is selected from N═C(R^{Z}), (R^{Z})C═N, (R^{Z})N—C(O), (R^{Z})₂C—C(O), N═N and (R^{Z})C═C(R⁶); J⁴-J³ is a group N═C(R^{Z}) or a group (R^{Z})N—CO; and R^{Z} is hydrogen or a substituent. The compounds of the formula (I) have PKA and PKB kinase inhibiting activity and are useful in the treatment of cancers.

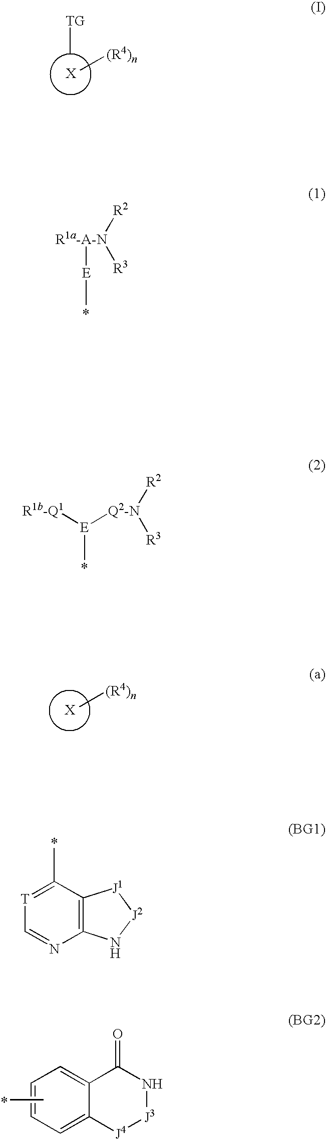

This invention relates to aryl- and heteroaryl-alkylamine compounds that inhibit or modulate the activity of protein kinase B (PKB) and protein kinase A (PKA), to the use of the compounds in the treatment or prophylaxis of disease states or conditions mediated by PKB and PKA, and to novel compounds having PKB and PKA inhibitory or modulating activity. Also provided are pharmaceutical compositions containing the compounds and novel chemical intermediates.

### BACKGROUND OF THE INVENTION

Protein kinases constitute a large family of structurally related enzymes that are responsible for the control of a wide variety of signal transduction processes within the cell (Hardie, G. and Hanks, S. (1995) The Protein Kinase Facts Book. I and II, Academic Press, San Diego, Calif.). The kinases may be categorized into families by the substrates they phosphorylate (e.g. protein-tyrosine, protein-serine/threonine, lipids, etc.). Sequence motifs have been identified that generally correspond to each of these kinase families (e.g. Hanks, S. K., Hunter, T., FASEB J., 9:576-596 (1995); Knighton, et al., Science, 253:407-414 (1991); Hiles, et al., Cell, 70:419-429 (1992); Kunz, et al., Cell, 73:585-596 (1993); Garcia-Bustos, et al., EMBO J, 13:2352-2361 (1994)).

Protein kinases may be characterized by their regulation mechanisms. These mechanisms include, for example, autophosphorylation, transphosphorylation by other kinases, protein-protein interactions, protein-lipid interactions, and protein-polynucleotide interactions. An individual protein kinase may be regulated by more than one mechanism.

Kinases regulate many different cell processes including, but not limited to, proliferation, differentiation, apoptosis, motility, transcription, translation and other signalling processes, by adding phosphate groups to target proteins. These phosphorylation events act as molecular on/off switches that can modulate or regulate the target protein biological function. Phosphorylation of target proteins occurs in response to a variety of extracellular signals (hormones, neurotransmitters, growth and differentiation factors, etc.), cell cycle events, environmental or nutritional stresses, etc. The appropriate protein kinase functions in signalling pathways to activate or inactivate (either directly or indirectly), for example, a metabolic enzyme, regulatory protein, receptor, cytoskeletal protein, ion channel or pump, or transcription factor. Uncontrolled signalling due to defective control of protein phosphorylation has been implicated in a number of diseases, including, for example, inflammation, cancer, allergy/asthma, diseases and conditions of the immune system, diseases and conditions of the central nervous system, and angiogenesis.

Apoptosis or programmed cell death is an important physiological process which removes cells no longer required by an organism. The process is important in early embryonic growth and development allowing the non-necrotic controlled breakdown, removal and recovery of cellular components. The removal of cells by apoptosis is also important in the maintenance of chromosomal and genomic integrity of growing cell populations. There are several known checkpoints in the cell growth cycle at which DNA damage and genomic integrity are carefully monitored. The response to the detection of anomalies at such checkpoints is to arrest the growth of such cells and initiate repair processes. If the damage or anomalies cannot be repaired then apoptosis is initiated by the damaged cell in order to prevent the propagation of faults and errors. Cancerous cells consistently contain numerous mutations, errors or rearrangements in their chromosomal DNA. It is widely believed that this occurs in part because the majority of tumours have a defect in one or more of the processes responsible for initiation of the apoptotic process. Normal control mechanisms cannot kill the cancerous cells and the chromosomal or DNA coding errors continue to be propagated. As a consequence restoring these pro-apoptotic signals or suppressing unregulated survival signals is an attractive means of treating cancer.

The signal transduction pathway containing the enzymes phosphatidylinositol 3-kinase (PI3K), PDK1 and PKB amongst others, has long been known to mediate increased resistance to apoptosis or survival responses in many cells. There is a substantial amount of data to indicate that this pathway is an important survival pathway used by many growth factors to suppress apoptosis. The enzyme PI3K is activated by a range of growth and survival factors e.g. EGF, PDGF and through the generation of polyphosphatidylinositols, initiates the activation of the downstream signalling events including the activity of the kinases PDK1 and protein kinase B (PKB) also known as Akt. This is also true in host tissues, e.g. vascular endothelial cells as well as neoplasias. PKB is a protein ser/thr kinase consisting of a kinase domain together with an N-terminal PH domain and C-terminal regulatory domain. The enzyme PKB itself is phosphorylated on Thr 308 by PDK1 and on Ser 473 by an as yet unidentified kinase. Full activation requires phosphorylation at both sites whilst association between PIP3 and the PH domain is required for anchoring of the enzyme to the cytoplasmic face of the lipid membrane providing optimal access to substrates.

Activated PKB in turn phosphorylates a range of substrates contributing to the overall survival response. Whilst we cannot be certain that we understand all of the factors responsible for mediating the PKB dependent survival response, some important actions are believed to be phosphorylation and inactivation of the pro-apoptotic factor BAD and caspase 9, phosphorylation of Forkhead transcription factors e.g. FKHR leading to their exclusion from the nucleus, and activation of the NfkappaB pathway by phosphorylation of upstream kinases in the cascade.

In addition to the anti-apoptotic and pro-survival actions of the PKB pathway, the enzyme also plays an important role in promoting cell proliferation. This action is again likely to be mediated via several actions, some of which are thought to be phosphorylation and inactivation of the cyclin dependent kinase inhibitor of p21^{Cip1/WAF1}, and phosphorylation and activation of mTOR, a kinase controlling several aspects of cell growth.

The phosphatase PTEN which dephosphorylates and inactivates polyphosphatidyl-inositols is a key tumour suppressor protein which normally acts to regulate the PI3K/PKB survival pathway. The significance of the PI3K/PKB pathway in tumorigenesis can be judged from the observation that PTEN is one of the most common targets of mutation in human tumours, with mutations in this phosphatase having been found in 50% or more of melanomas (Guldberg et al 1997, Cancer Research 57, 3660-3663) and advanced prostate cancers (Cairns et al 1997 Cancer Research 57, 4997). These observations and others suggest that a wide range of tumour types are dependent on the enhanced PKB activity for growth and survival and would respond therapeutically to appropriate inhibitors of PKB.

There are 3 closely related isoforms of PKB called alpha, beta and gamma, which genetic studies suggest have distinct but overlapping functions. Evidence suggests that they can all independently play a role in cancer. For example PKB beta has been found to be over-expressed or activated in 10-40% of ovarian and pancreatic cancers (Bellacosa et al 1995, Int. J. Cancer 64, 280-285; Cheng et al 1996, PNAS 93, 3636-3641; Yuan et al 2000, Oncogene 19, 2324-2330), PKB alpha is amplified in human gastric, prostate and breast cancer (Staal 1987, PNAS 84, 5034-5037; Sun et al 2001, Am. J. Pathol. 159, 431-437) and increased PKB gamma activity has been observed in steroid independent breast and prostate cell lines (Nakatani et al 1999, J. Biol. Chem. 274, 21528-21532).

The PKB pathway also functions in the growth and survival of normal tissues and may be regulated during normal physiology to control cell and tissue function. Thus disorders associated with undesirable proliferation and survival of normal cells and tissues may also benefit therapeutically from treatment with a PKB inhibitor. Examples of such disorders are disorders of immune cells associated with prolonged expansion and survival of cell population leading to a prolonged or up regulated immune response. For example, T and B lymphocyte response to cognate antigens or growth factors such as interleukin-2 activates the PI3K/PKB pathway and is responsible for maintaining the survival of the antigen specific lymphocyte clones during the immune response. Under conditions in which lymphocytes and other immune cells are responding to inappropriate self or foreign antigens, or in which other abnormalities lead to prolonged activation, the PKB pathway contributes an important survival signal preventing the normal mechanisms by which the immune response is terminated via apoptosis of the activated cell population. There is a considerable amount of evidence demonstrating the expansion of lymphocyte populations responding to self antigens in autoimmune conditions such as multiple sclerosis and arthritis. Expansion of lymphocyte populations responding inappropriately to foreign antigens is a feature of another set of conditions such as allergic responses and asthma. In summary inhibition of PKB could provide a beneficial treatment for immune disorders.

Other examples of inappropriate expansion, growth, proliferation, hyperplasia and survival of normal cells in which PKB may play a role include but are not limited to atherosclerosis, cardiac myopathy and glomerulonephritis.

In addition to the role in cell growth and survival, the PKB pathway functions in the control of glucose metabolism by insulin. Available evidence from mice deficient in the alpha and beta isoforms of PKB suggests that this action is mediated by the beta isoform. As a consequence, modulators of PKB activity may also find utility in diseases in which there is a dysfunction of glucose metabolism and energy storage such as diabetes, metabolic disease and obesity.

Cyclic AMP-dependent protein kinase (PKA) is a serine/threonine protein kinase that phosphorylates a wide range of substrates and is involved in the regulation of many cellular processes including cell growth, cell differentiation, ion-channel conductivity, gene transcription and synaptic release of neurotransmitters. In its inactive form, the PKA holoenzyme is a tetramer comprising two regulatory subunits and two catalytic subunits.

PKA acts as a link between G-protein mediated signal transduction events and the cellular processes that they regulate. Binding of a hormone ligand such as glucagon to a transmembrane receptor activates a receptor-coupled G-protein (GTP-binding and hydrolyzing protein). Upon activation, the alpha subunit of the G protein dissociates and binds to and activates adenylate cyclase, which in turn converts ATP to cyclic-AMP (cAMP). The cAMP thus produced then binds to the regulatory subunits of PKA leading to dissociation of the associated catalytic subunits. The catalytic subunits of PKA, which are inactive when associated with the regulatory sub-units, become active upon dissociation and take part in the phosphorylation of other regulatory proteins.

For example, the catalytic sub-unit of PKA phosphorylates the kinase Phosphorylase Kinase which is involved in the phosphorylation of Phosphorylase, the enzyme responsible for breaking down glycogen to release glucose. PKA is also involved in the regulation of glucose levels by phosphorylating and deactivating glycogen synthase. Thus, modulators of PKA activity (which modulators may increase or decrease PKA activity) may be useful in the treatment or management of diseases in which there is a dysfunction of glucose metabolism and energy storage such as diabetes, metabolic disease and obesity.

PKA has also been established as an acute inhibitor of T cell activation. Anndahl et al, have investigated the possible role of PKA type I in HIV-induced T cell dysfunction on the basis that T cells from HIV-infected patients have increased levels of cAMP and are more sensitive to inhibition by cAMP analogues than are normal T cells. From their studies, they concluded that increased activation of PKA type I may contribute to progressive T cell dysfunction in HIV infection and that PKA type I may therefore be a potential target for immunomodulating therapy.—Aandahl, E. M., Aukrust, P., Skålhegg, B. S., Müller, F., Frøland, S. S., Hansson, V., Taskén, K. Protein kinase A type I antagonist restores immune responses of T cells from HIV-infected patients. FASEB J. 12, 855-862 (1998).

It has also been recognised that mutations in the regulatory sub-unit of PKA can lead to hyperactivation in endocrine tissue.

Because of the diversity and importance of PKA as a messenger in cell regulation, abnormal responses of cAMP can lead to a variety of human diseases such as irregular cell growth and proliferation (Stratakis, C. A.; Cho-Chung, Y. S.; Protein Kinase A and human diseases. Trends Endrocri. Metab. 2002, 13, 50-52). Over-expression of PKA has been observed in a variety of human cancer cells including those from ovarian, breast and colon patients. Inhibition of PKA would therefore be an approach to treatment of cancer (Li, Q.; Zhu, G-D.; Current Topics in Medicinal Chemistry, 2002, 2, 939-971).

For a review of the role of PKA in human disease, see for example, Protein Kinase A and Human Disease, Edited by Constantine A. Stratakis, Annals of the New York Academy of Sciences, Volume 968, 2002, ISBN 1-57331-412-9.

hERG

In the late 1990s a number of drugs, approved by the US FDA, had to be withdrawn from sale in the US when it was discovered they were implicated in deaths caused by heart malfunction. It was subsequently found that a side effect of these drugs was the development of arrhythmias caused by the blocking of hERG channels in heart cells. The hERG channel is one of a family of potassium ion channels the first member of which was identified in the late 1980s in a mutant Drosophila melanogaster fruitfly (see Jan, L. Y. and Jan, Y. N. (1990). A Superfamily of Ion Channels. Nature, 345(6277):672). The biophysical properties of the hERG potassium ion channel are described in Sanguinetti, M. C., Jiang, C., Curran, M. E., and Keating, M. T. (1995). A Mechanistic Link Between an Inherited and an Acquired Cardiac Arrhythmia: HERG encodes the Ikr potassium channel. Cell, 81:299-307, and Trudeau, M. C., Warmke, J. W., Ganetzky, B., and Robertson, G. A. (1995). HERG, a Human Inward Rectifier in the Voltage-Gated Potassium Channel Family. Science, 269:92-95.

### PRIOR ART

Several classes of compounds have been disclosed as having PKA and PKB inhibitory activity.

For example, a class of isoquinolinyl-sulphonamido-diamines having PKB inhibitory activity is disclosed in WO 01/91754 (Yissum).

WO 2005/061463 (Astex) discloses pyrazole compounds having PKB and PKA inhibiting activity.

### SUMMARY OF THE INVENTION

The invention provides compounds that have protein kinase B (PKB) and protein A (PKA) inhibiting or modulating activity, and which it is envisaged will be useful in preventing or treating disease states or conditions mediated by PKB or PKA.

In a first aspect, the invention provides a compound of the formula (I):

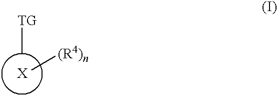

or a salt, solvate, tautomer or N-oxide thereof;
wherein TG is selected from:
(1) a group:

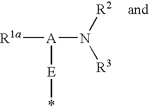

(2) a group:

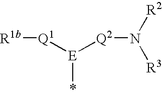

wherein the asterisk (*) represents the point of attachment of the group E to the group X;
A is a saturated hydrocarbon linker group containing from 1 to 7 carbon atoms, the linker group having a maximum chain length of 5 atoms extending between R^{1a} and NR²R³ and a maximum chain length of 4 atoms extending between E and NR²R³, wherein one of the carbon atoms in the linker group may optionally be replaced by an oxygen or nitrogen atom; and wherein the carbon atoms of the linker group A may optionally bear one or more substituents selected from oxo, fluorine and hydroxy, provided that the hydroxy group when present is not located at a carbon atom α with respect to the NR²R³ group and provided that the oxo group when present is located at a carbon atom α with respect to the NR²R³ group;
E is a monocyclic or bicyclic carbocyclic or heterocyclic group;
the group X is a bicyclic heterocyclic group having 8 to 12 ring members of which up to 5 are heteroatoms selected from O, N and S;
R^{1a} is an aryl or heteroaryl group;
R^{1b} is hydrogen or a group R^{1a};
R² and R³ are independently selected from hydrogen, C₁₋₄ hydrocarbyl and C₁₋₄ acyl wherein the hydrocarbyl and acyl moieties are optionally substituted by one or more substituents selected from fluorine, hydroxy, amino, methylamino, dimethylamino and methoxy;
or R² and R³ together with the nitrogen atom to which they are attached form a cyclic group selected from an imidazole group and a saturated monocyclic heterocyclic group having 4-7 ring members and optionally containing a second heteroatom ring member selected from O and N;
or one of R² and R³ together with the nitrogen atom to which they are attached and one or more atoms from the linker group A form a saturated monocyclic heterocyclic group having 4-7 ring members and optionally containing a second heteroatom ring member selected from O and N;
or NR²R³ and the carbon atom of linker group A to which it is attached together form a cyano group;
n is 0 to 4;
each R⁴ is independently selected from oxo; halogen; C₁₋₆ hydrocarbyl optionally substituted by halogen, hydroxy or C₁₋₂ alkoxy; cyano; C₁₋₆ hydrocarbyloxy optionally substituted by halogen, hydroxy or C₁₋₂ alkoxy; CONH₂; CONHR⁹; CF₃; NH₂; NHCOR⁹; NHCONHR⁹; and NHR⁹;
R⁹ is a group R^{9a} or (CH₂)R^{9a}, wherein R^{9a} is a monocyclic or bicyclic group which may be carbocyclic or heterocyclic;
the carbocyclic group or heterocyclic group R^{9a} being optionally substituted by one or more substituents selected from halogen, hydroxy, trifluoromethyl, cyano, nitro, carboxy, amino, mono- or di-C₁₋₄ hydrocarbylamino; a group R^{a}-R^{b} wherein R^{a} is a bond, O, CO, X¹C(X²), C(X²)X¹, X¹C(X²)X¹, S, SO, SO₂, NR^{c}, SO₂NR^{c} or NR^{c}SO₂; and R^{b} is selected from hydrogen, heterocyclic groups having from 3 to 12 ring members, and a C₁₋₈ hydrocarbyl group optionally substituted by one or more substituents selected from hydroxy, oxo, halogen, cyano, nitro, carboxy, amino, mono- or di-C₁₋₄ hydrocarbylamino, carbocyclic and heterocyclic groups having from 3 to 12 ring members and wherein one or more carbon atoms of the C₁₋₈ hydrocarbyl group may optionally be replaced by O, S, SO, SO₂, NR^{c}, X¹C(X²), C(X²)X¹ or X¹C(X²)X¹;
R^{c} is selected from hydrogen and C₁₋₄ hydrocarbyl; and
X¹ is O, S or NR^{c} and X² is ═O, ═S or ═NR^{c};
Q¹ is a bond or a saturated hydrocarbon linker group containing from 1 to 3 carbon atoms, wherein one of the carbon atoms in the linker group may optionally be replaced by an oxygen or nitrogen atom, or an adjacent pair of carbon atoms may be replaced by CONR^{q} or NR^{q}CO where R^{q} is hydrogen, C₁₋₄ alkyl or cyclopropyl, or R^{q} is a C₁₋₄ alkylene chain that links to R^{1b} or to another carbon atom of Q¹ to form a cyclic moiety; and wherein the carbon atoms of the linker group Q¹ may optionally bear one or more substituents selected from fluorine and hydroxy;
Q² is a bond or a saturated hydrocarbon linker group containing from 1 to 3 carbon atoms, wherein one of the carbon atoms in the linker group may optionally be replaced by an oxygen or nitrogen atom; and wherein the carbon atoms of the linker group may optionally bear one or more substituents selected from fluorine and hydroxy, provided that the hydroxy group when present is not located at a carbon atom α with respect to the NR²R³ group; and provided that when E is aryl or heteroaryl, then Q² is other than a bond;
and further provided that the moiety

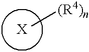

is other than a group (BG1) or (BG2);

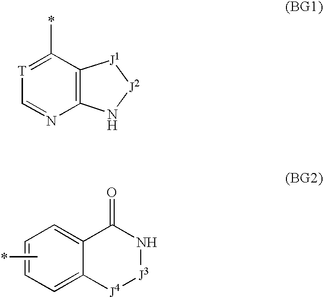

wherein (BG1) and (BG2) are each optionally substituted;
the asterisk (*) represents the point of attachment of the group E;
T is N or a group CR^{z};
J¹-J² represents a group selected from N═C(R^{z}), (R^{z})C═N, (R^{z})N—C(O), (R^{z})₂C—C(O), N═N and (R^{z})C═C(R⁶);
J⁴-J³ is a group N═C(R^{z}) or a group (R^{z})N—CO; and
R^{z} is hydrogen or a substituent.

Compounds containing the groups BG1 and BG2 are disclosed in our earlier International patent applications PCT/GB2005/004115, PCT/GB2005/004119 and PCT/GB2005/004323 (the contents of which are incorporated herein by reference) and such compounds are explicitly excluded from the scope of formula (I) of the present application.

The invention further provides:
A compound of the formula (I), (II), (IIa), (IIb), (IIc), (III), (IV), (V), (VI), (VII), (VIII) and (IX) or any sub-group thereof as defined herein for use in the prophylaxis or treatment of a disease state or condition mediated by protein kinase B.
The use of a compound of formula (I), (II), (IIa), (IIb), (IIc), (III), (IV), (V), (VI), (VII), (VIII), (IX) or any sub-group thereof as defined herein for the manufacture of a medicament for the prophylaxis or treatment of a disease state or condition mediated by protein kinase B.
A method for the prophylaxis or treatment of a disease state or condition mediated by protein kinase B, which method comprises administering to a subject in need thereof a compound of the formula (I), (II), (IIa), (IIb), (IIc), (III), (IV), (V), (VI), (VII), (VIII), (IX) or any sub-group thereof as defined herein.
A compound of the formula (I), (II), (IIa), (IIb), (IIc), (III), (IV), (V), (VI), (VII), (VIII) and (IX) or any sub-group thereof as defined herein for use in treating a disease or condition comprising or arising from abnormal cell growth or abnormally arrested cell death in a mammal.
The use of a compound of formula (I), (II), (IIa), (IIb), (IIc), (III), (IV), (V), (VI), (VII), (VIII), (IX) or any sub-group thereof as defined herein for the manufacture of a medicament for treating a disease or condition comprising or arising from abnormal cell growth or abnormally arrested cell death in a mammal.
A method for treating a disease or condition comprising or arising from abnormal cell growth or abnormally arrested cell death in a mammal, the method comprising administering to the mammal a compound of the formula (I), (II), (IIa), (IIb), (IIc), (III), (IV), (V), (VI), (VII), (VIII), (IX) or any sub-group thereof as defined herein in an amount effective to inhibit protein kinase B activity.
A compound of the formula (I), (II), (IIa), (IIb), (IIc), (III), (IV), (V), (VI), (VII), (VIII) and (IX) or any sub-group thereof as defined herein for use in inhibiting protein kinase B.
A method of inhibiting protein kinase B, which method comprises contacting the kinase with a kinase-inhibiting compound of the formula (I), (II), (IIa), (IIb), (IIc), (III), (IV), (V), (VI), (VII), (VIII), (IX) or any sub-group thereof as defined herein.
A compound of the formula (I), (II), (IIa), (IIb), (IIc), (III), (IV), (V), (VI), (VII), (VIII) and (IX) or any sub-group thereof as defined herein for use in modulating a cellular process (for example cell division) by inhibiting the activity of a protein kinase B.
A method of modulating a cellular process (for example cell division) by inhibiting the activity of a protein kinase B using a compound of the formula (I), (II), (IIa), (IIb), (IIc), (III), (IV), (V), (VI), (VII), (VIII), (IX) or any sub-group thereof as defined herein.
A compound of the formula (I), (II), (IIa), (IIb), (IIc), (III), (IV), (V), (VI), (VII), (VIII), (IX) or any sub-group or embodiment thereof as defined herein for use in the prophylaxis or treatment of a disease state or condition mediated by protein kinase A.
The use of a compound of formula (I), (II), (IIa), (IIb), (IIc), (III), (IV), (V), (VI), (VII), (VIII), (IX) or any sub-group or embodiment thereof as defined herein for the manufacture of a medicament for the prophylaxis or treatment of a disease state or condition mediated by protein kinase A.
A method for the prophylaxis or treatment of a disease state or condition mediated by protein kinase A, which method comprises administering to a subject in need thereof a compound of the formula (I), (II), (IIa), (IIb), (IIc), (III), (IV), (V), (VI), (VII), (VIII), (IX) or any sub-group or embodiment thereof as defined herein.
A method for treating a disease or condition comprising or arising from abnormal cell growth or abnormally arrested cell death in a mammal, the method comprising administering to the mammal a compound of the formula (I), (II), (IIa), (IIb), (IIc), (III), (IV), (V), (VI), (VII), (VIII), (IX) or any sub-group or embodiment thereof as defined herein in an amount effective to inhibit protein kinase A activity.
A method of inhibiting protein kinase A, which method comprises contacting the kinase with a kinase-inhibiting compound of the formula (I), (II), (IIa), (IIb), (IIc), (III), (IV), (V), (VI), (VII), (VIII), (IX) or any sub-group or embodiment thereof as defined herein.
A method of modulating a cellular process (for example cell division) by inhibiting the activity of a protein kinase A using a compound of the formula (I), (II), (IIa), (IIb), (IIc), (III), (IV), (V), (VI), (VII), (VIII), (IX) or any sub-group or embodiment thereof as defined herein.
A method for treating a disease or condition comprising or arising from abnormal cell growth in a mammal, which method comprises administering to the mammal a compound of the formula (I), (II), (IIa), (IIb), (IIc), (III), (IV), (V), (VI), (VII), (VIII), (IX) or any sub-group thereof as defined herein in an amount effective in inhibiting abnormal cell growth or abnormally arrested cell death.
A compound of the formula (I), (II), (IIa), (IIb), (IIc), (III), (IV), (V), (VI), (VII), (VIII), (IX) or any sub-group or embodiment thereof as defined herein for use in alleviating or reducing the incidence of a disease or condition comprising or arising from abnormal cell growth or abnormally arrested cell death in a mammal.
A method for alleviating or reducing the incidence of a disease or condition comprising or arising from abnormal cell growth or abnormally arrested cell death in a mammal, which method comprises administering to the mammal a compound of the formula (I), (II), (IIa), (IIb), (IIc), (III), (IV), (V), (VI), (VII), (VIII), (IX) or any sub-group thereof as defined herein in an amount effective in inhibiting abnormal cell growth.
A pharmaceutical composition comprising a novel compound of the formula (I), (II), (IIa), (IIb), (IIc), (III), (IV), (V), (VI), (VII), (VIII), (IX) or any sub-group thereof as defined herein and a pharmaceutically acceptable carrier.
A compound of the formula (I), (II), (IIa), (IIb), (IIc), (III), (IV), (V), (VI), (VII), (VIII), (IX) or any sub-group thereof as defined herein for use in medicine.
The use of a compound of the formula (I), (II), (IIa), (IIb), (IIc), (III), (IV), (V), (VI), (VII), (VIII), (IX) or any sub-group thereof as defined herein for the manufacture of a medicament for the prophylaxis or treatment of any one of the disease states or conditions disclosed herein.
A method for the treatment or prophylaxis of any one of the disease states or conditions disclosed herein, which method comprises administering to a patient (e.g. a patient in need thereof) a compound (e.g. a therapeutically effective amount) of the formula (I), (II), (IIa), (IIb), (IIc), (III), (IV), (V), (VI), (VII), (VIII), (IX) or any sub-group thereof as defined herein.
A method for alleviating or reducing the incidence of a disease state or condition disclosed herein, which method comprises administering to a patient (e.g. a patient in need thereof) a compound (e.g. a therapeutically effective amount) of the formula (I), (II), (IIa), (IIb), (IIc), (III), (IV), (V), (VI), (VII), (VIII), (IX) or any sub-group thereof as defined herein.
A compound of the formula (I), (II), (IIa), (IIb), (IIc), (III), (IV), (V), (VI), (VII), (VIII), (IX) or any sub-group or embodiment thereof as defined herein for use in the treatment or prophylaxis of a disease state or condition in a patient who has been screened and has been determined as suffering from, or being at risk of suffering from, a disease or condition which would be susceptible to treatment with a compound having activity against protein kinase B.
A method for the diagnosis and treatment of a disease state or condition mediated by protein kinase B, which method comprises (i) screening a patient to determine whether a disease or condition from which the patient is or may be suffering is one which would be susceptible to treatment with a compound having activity against protein kinase B; and (ii) where it is indicated that the disease or condition from which the patient is thus susceptible, thereafter administering to the patient a compound of the formula (I), (II), (IIa), (IIb), (IIc), (III), (IV), (V), (VI), (VII), (VIII), (IX) or any sub-group thereof as defined herein.
The use of a compound of the formula (I), (II), (IIa), (IIb), (IIc), (III), (IV), (V), (VI), (VII), (VIII), (IX) or any sub-group thereof as defined herein for the manufacture of a medicament for the treatment or prophylaxis of a disease state or condition in a patient who has been screened and has been determined as suffering from, or being at risk of suffering from, a disease or condition which would be susceptible to treatment with a compound having activity against protein kinase B.
A method for the diagnosis and treatment of a disease state or condition mediated by protein kinase A, which method comprises (i) screening a patient to determine whether a disease or condition from which the patient is or may be suffering is one which would be susceptible to treatment with a compound having activity against protein kinase A; and (ii) where it is indicated that the disease or condition from which the patient is thus susceptible, thereafter administering to the patient a compound of the formula (I), (II), (IIa), (IIb), (IIc), (III), (IV), (V), (VI), (VII), (VIII), (IX) or any sub-group or embodiment thereof as defined herein.
The use of a compound of the formula (I), (II), (IIa), (IIb), (IIc), (III), (IV), (V), (VI), (VII), (VIII), (IX) or any sub-group or embodiment thereof as defined herein for the manufacture of a medicament for the treatment or prophylaxis of a disease state or condition in a patient who has been screened and has been determined as suffering from, or being at risk of suffering from, a disease or condition which would be susceptible to treatment with a compound having activity against protein kinase A.
A compound of the formula (I), (II), (IIa), (IIb), (IIc), (III), (IV), (V), (VI), (VII), (VIII), (IX) or any sub-group or embodiment thereof as defined herein for use in the treatment or prophylaxis of a disease state or condition in a patient who has been screened and has been determined as suffering from, or being at risk of suffering from, a disease or condition which would be susceptible to treatment with a compound having activity against protein kinase A.

### General Preferences and Definitions

The following general preferences and definitions shall apply to each of the moieties A, E, Q¹, Q², and R^{1a} to R⁹, and any sub-definition, sub-group or embodiment thereof, unless the context indicates otherwise.

Any references to Formula (I) herein shall be taken also to refer to formulae (II), (IIa), (IIb), (IIc), (III), (IV), (V), (VI), (VII), (VIII), (IX) and any other sub-group of compounds within formula (I) unless the context requires otherwise.

In this application, the moiety:

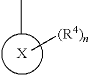

may be referred to for convenience as “the bicyclic group X” or “the bicyclic group”.

References to “carbocyclic” and “heterocyclic” groups as used herein shall, unless the context indicates otherwise, include both aromatic and non-aromatic ring systems. In general, such groups may be monocyclic or bicyclic and may contain, for example, 3 to 12 ring members, more usually 5 to 10 ring members. Examples of monocyclic groups are groups containing 3, 4, 5, 6, 7, and 8 ring members, more usually 3 to 7, and preferably 5 or 6 ring members. Examples of bicyclic groups are those containing 8, 9, 10, 11 and 12 ring members, and more usually 9 or 10 ring members.

The carbocyclic or heterocyclic groups can be aryl or heteroaryl groups having from 5 to 12 ring members, more usually from 5 to 10 ring members. The term “aryl” as used herein refers to a carbocyclic group having aromatic character and the term “heteroaryl” is used herein to denote a heterocyclic group having aromatic character. The terms “aryl” and “heteroaryl” embrace polycyclic (e.g. bicyclic) ring systems wherein one or more rings are non-aromatic, provided that at least one ring is aromatic. In such polycyclic systems, the group may be attached by the aromatic ring, or by a non-aromatic ring. The aryl or heteroaryl groups can be monocyclic or bicyclic groups and can be unsubstituted or substituted with one or more substituents, for example one or more groups R¹⁰ as defined herein.

The term non-aromatic group embraces unsaturated ring systems without aromatic character, partially saturated and fully saturated carbocyclic and heterocyclic ring systems. The terms “unsaturated” and “partially saturated” refer to rings wherein the ring structure(s) contains atoms sharing more than one valence bond i.e. the ring contains at least one multiple bond e.g. a C═C, C≡C or N═C bond. The term “fully saturated” refers to rings where there are no multiple bonds between ring atoms. Saturated carbocyclic groups include cycloalkyl groups as defined below. Partially saturated carbocyclic groups include cycloalkenyl groups as defined below, for example cyclopentenyl, cycloheptenyl and cyclooctenyl.

Examples of heteroaryl groups are monocyclic and bicyclic groups containing from five to twelve ring members, and more usually from five to ten ring members. The heteroaryl group can be, for example, a five membered or six membered monocyclic ring or a bicyclic structure formed from fused five and six membered rings or two fused six membered rings. Each ring may contain up to about four heteroatoms typically selected from nitrogen, sulphur and oxygen. Typically the heteroaryl ring will contain up to 3 heteroatoms, more usually up to 2, for example a single heteroatom. In one embodiment, the heteroaryl ring contains at least one ring nitrogen atom. The nitrogen atoms in the heteroaryl rings can be basic, as in the case of an imidazole or pyridine, or essentially non-basic as in the case of an indole or pyrrole nitrogen. In general the number of basic nitrogen atoms present in the heteroaryl group, including any amino group substituents of the ring, will be less than five.

Examples of five membered heteroaryl groups include but are not limited to pyrrole, furan, thiophene, imidazole, furazan, oxazole, oxadiazole, oxatriazole, isoxazole, thiazole, isothiazole, pyrazole, triazole and tetrazole groups.

Examples of six membered heteroaryl groups include but are not limited to pyridine, pyrazine, pyridazine, pyrimidine and triazine.

A bicyclic heteroaryl group may be, for example, a group selected from:
a) a benzene ring fused to a 5- or 6-membered ring containing 1, 2 or 3 ring heteroatoms;
b) a pyridine ring fused to a 5- or 6-membered ring containing 1, 2 or 3 ring heteroatoms;
c) a pyrimidine ring fused to a 5- or 6-membered ring containing 1 or 2 ring heteroatoms;
d) a pyrrole ring fused to a 5- or 6-membered ring containing 1, 2 or 3 ring heteroatoms;
e) a pyrazole ring fused to a 5- or 6-membered ring containing 1 or 2 ring heteroatoms;
f) a pyrazine ring fused to a 5- or 6-membered ring containing 1 or 2 ring heteroatoms;
g) an imidazole ring fused to a 5- or 6-membered ring containing 1 or 2 ring heteroatoms;
h) an oxazole ring fused to a 5- or 6-membered ring containing 1 or 2 ring heteroatoms;
i) an isoxazole ring fused to a 5- or 6-membered ring containing 1 or 2 ring heteroatoms;
j) a thiazole ring fused to a 5- or 6-membered ring containing 1 or 2 ring heteroatoms;
k) an isothiazole ring fused to a 5- or 6-membered ring containing 1 or 2 ring heteroatoms;
l) a thiophene ring fused to a 5- or 6-membered ring containing 1, 2 or 3 ring heteroatoms;
m) a furan ring fused to a 5- or 6-membered ring containing 1, 2 or 3 ring heteroatoms;
n) a cyclohexyl ring fused to a 5- or 6-membered ring containing 1, 2 or 3 ring heteroatoms; and
o) a cyclopentyl ring fused to a 5- or 6-membered ring containing 1, 2 or 3 ring heteroatoms.

Examples of bicyclic heteroaryl groups containing a six membered ring fused to a five membered ring include but are not limited to benzfuran, benzthiophene, benzimidazole, benzoxazole, benzisoxazole, benzthiazole, benzisothiazole, isobenzofuran, indole, isoindole, indolizine, indoline, isoindoline, purine (e.g., adenine, guanine), indazole, benzodioxole and pyrazolopyridine groups.

Examples of bicyclic heteroaryl groups containing two fused six membered rings include but are not limited to quinoline, isoquinoline, chroman, thiochroman, chromene, isochromene, chroman, isochroman, benzodioxan, quinolizine, benzoxazine, benzodiazine, pyridopyridine, quinoxaline, quinazoline, cinnoline, phthalazine, naphthyridine and pteridine groups.

Examples of polycyclic aryl and heteroaryl groups containing an aromatic ring and a non-aromatic ring include tetrahydronaphthalene, tetrahydroisoquinoline, tetrahydroquinoline, dihydrobenzthiene, dihydrobenzofuran, 2,3-dihydro-benzo[1,4]dioxine, benzo[1,3]dioxole, 4,5,6,7-tetrahydrobenzofuran, indoline and indane groups.

Examples of carbocyclic aryl groups include phenyl, naphthyl, indenyl, and tetrahydronaphthyl groups.

Examples of non-aromatic heterocyclic groups are groups having from 3 to 12 ring members, more usually 5 to 10 ring members. Such groups can be monocyclic or bicyclic, for example, and typically have from 1 to 5 heteroatom ring members (more usually 1, 2, 3 or 4 heteroatom ring members), usually selected from nitrogen, oxygen and sulphur.

The heterocylic groups can contain, for example, cyclic ether moieties (e.g. as in tetrahydrofuran and dioxane), cyclic thioether moieties (e.g. as in tetrahydrothiophene and dithiane), cyclic amine moieties (e.g. as in pyrrolidine), cyclic sulphones (e.g. as in sulpholane and sulpholene), cyclic sulphoxides, cyclic sulphonamides and combinations thereof (e.g. thiomorpholine). Other examples of non-aromatic heterocyclic groups include cyclic amide moieties (e.g. as in pyrrolidone) and cyclic ester moieties (e.g. as in butyrolactone).

Examples of monocyclic non-aromatic heterocyclic groups include 5-, 6- and 7-membered monocyclic heterocyclic groups. Particular examples include morpholine, thiomorpholine and its S-oxide and S,S-dioxide (particularly thiomorpholine), piperidine (e.g. 1-piperidinyl, 2-piperidinyl 3-piperidinyl and 4-piperidinyl), N-alkyl piperidines such as N-methyl piperidine, piperidone, pyrrolidine (e.g. 1-pyrrolidinyl, 2-pyrrolidinyl and 3-pyrrolidinyl), pyrrolidone, azetidine, pyran (2H-pyran or 4H-pyran), dihydrothiophene, dihydropyran, dihydrofuran, dihydrothiazole, tetrahydrofuran, tetrahydrothiophene, dioxane, tetrahydropyran (e.g. 4-tetrahydro pyranyl), imidazoline, imidazolidinone, oxazoline, thiazoline, 2-pyrazoline, pyrazolidine, piperazone, piperazine, and N-alkyl piperazines such as N-methyl piperazine, N-ethyl piperazine and N-isopropylpiperazine.

One sub-group of monocyclic non-aromatic heterocyclic groups includes morpholine, piperidine (e.g. 1-piperidinyl, 2-piperidinyl 3-piperidinyl and 4-piperidinyl), piperidone, pyrrolidine (e.g. 1-pyrrolidinyl, 2-pyrrolidinyl and 3-pyrrolidinyl), pyrrolidone, pyran (2H-pyran or 4H-pyran), dihydrothiophene, dihydropyran, dihydrofuran, dihydrothiazole, tetrahydrofuran, tetrahydrothiophene, dioxane, tetrahydropyran (e.g. 4-tetrahydro pyranyl), imidazoline, imidazolidinone, oxazoline, thiazoline, 2-pyrazoline, pyrazolidine, piperazone, piperazine, and N-alkyl piperazines such as N-methyl piperazine. In general, preferred non-aromatic heterocyclic groups include piperidine, pyrrolidine, azetidine, morpholine, piperazine and N-alkyl piperazines. A further particular example of a non-aromatic heterocyclic group, which also forms part of the above group of preferred non-aromatic heterocyclic groups, is azetidine.

Examples of non-aromatic carbocyclic groups include cycloalkane groups such as cyclohexyl and cyclopentyl, cycloalkenyl groups such as cyclopentenyl, cyclohexenyl, cycloheptenyl and cyclooctenyl, as well as cyclohexadienyl, cyclooctatetraene, tetrahydronaphthenyl and decalinyl.

Where reference is made herein to carbocyclic and heterocyclic groups, the carbocyclic or heterocyclic ring can, unless the context indicates otherwise, be unsubstituted or substituted by one or more substituent groups R¹⁰ selected from halogen, hydroxy, trifluoromethyl, cyano, nitro, carboxy, amino, mono- or di-C₁₋₄ hydrocarbylamino, carbocyclic and heterocyclic groups having from 3 to 12 ring members; a group R^{a}-R^{b} wherein R^{a} is a bond, O, CO, X¹C(X²), C(X²)X¹, X¹C(X²)X¹, S, SO, SO₂, NR^{c}, SO₂NR^{c} or NR^{c}SO₂; and R^{b} is selected from hydrogen, carbocyclic and heterocyclic groups having from 3 to 12 ring members, and a C₁₋₈ hydrocarbyl group optionally substituted by one or more substituents selected from hydroxy, oxo, halogen, cyano, nitro, carboxy, amino, mono- or di-C₁₋₄ hydrocarbylamino, carbocyclic and heterocyclic groups having from 3 to 12 ring members and wherein one or more carbon atoms of the C₁₋₈ hydrocarbyl group may optionally be replaced by O, S, SO, SO₂, NR^{c}, X¹C(X²), C(X²)X¹ or X¹C(X²)X¹;
R^{c} is selected from hydrogen and C₁₋₄ hydrocarbyl; and
X¹ is O, S or NR^{c} and X² is ═O, ═S or ═NR^{c}.

Where the substituent group R¹⁰ comprises or includes a carbocyclic or heterocyclic group, the said carbocyclic or heterocyclic group may be unsubstituted or may itself be substituted with one or more further substituent groups R¹⁰. In one sub-group of compounds of the formula (I), such further substituent groups R¹⁰ may include carbocyclic or heterocyclic groups, which are typically not themselves further substituted. In another sub-group of compounds of the formula (I), the said further substituents do not include carbocyclic or heterocyclic groups but are otherwise selected from the groups listed above in the definition of R¹⁰.

The substituents R¹⁰ may be selected such that they contain no more than 20 non-hydrogen atoms, for example, no more than 15 non-hydrogen atoms, e.g. no more than 12, or 10, or 9, or 8, or 7, or 6, or 5 non-hydrogen atoms.

Where the carbocyclic and heterocyclic groups have a pair of substituents on adjacent ring atoms, the two substituents may be linked so as to form a cyclic group. For example, an adjacent pair of substituents on adjacent carbon atoms of a ring may be linked via one or more heteroatoms and optionally substituted alkylene groups to form a fused oxa-, dioxa-, aza-, diaza- or oxa-aza-cycloalkyl group. Examples of such linked substituent groups include:

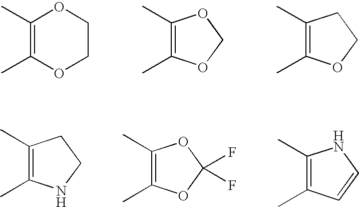

Examples of halogen substituents include fluorine, chlorine, bromine and iodine. Fluorine and chlorine are particularly preferred.

In the definition of the compounds of the formula (I) above and as used hereinafter, the term “hydrocarbyl” is a generic term encompassing aliphatic, alicyclic and aromatic groups having an all-carbon backbone, except where otherwise stated. In certain cases, as defined herein, one or more of the carbon atoms making up the carbon backbone may be replaced by a specified atom or group of atoms. Examples of hydrocarbyl groups include alkyl, cycloalkyl, cycloalkenyl, carbocyclic aryl, alkenyl, alkynyl, cycloalkylalkyl, cycloalkenylalkyl, and carbocyclic aralkyl, aralkenyl and aralkenyl groups. Such groups can be unsubstituted or, where stated, can be substituted by one or more substituents as defined herein. The examples and preferences expressed below apply to each of the hydrocarbyl substituent groups or hydrocarbyl-containing substituent groups referred to in the various definitions of substituents for compounds of the formula (I) unless the context indicates otherwise.

Generally by way of example, the hydrocarbyl groups can have up to eight carbon atoms, unless the context requires otherwise. Within the sub-set of hydrocarbyl groups having 1 to 8 carbon atoms, particular examples are C₁₋₆ hydrocarbyl groups, such as C₁₋₄ hydrocarbyl groups (e.g. C₁₋₃ hydrocarbyl groups or C₁₋₂ hydrocarbyl groups), specific examples being any individual value or combination of values selected from C₁, C₂, C₃, C₄, C₅, C₆, C₇ and C₈ hydrocarbyl groups.

The term “alkyl” covers both straight chain and branched chain alkyl groups. Examples of alkyl groups include methyl, ethyl, propyl, isopropyl, n-butyl, isobutyl, tert-butyl, n-pentyl, 2-pentyl, 3-pentyl, 2-methyl butyl, 3-methyl butyl, and n-hexyl and its isomers. Within the sub-set of alkyl groups having 1 to 8 carbon atoms, particular examples are C₁₋₆ alkyl groups, such as C₁₋₄ alkyl groups (e.g. C₁₋₃ alkyl groups or C₁₋₂ alkyl groups).

Examples of cycloalkyl groups are those derived from cyclopropane, cyclobutane, cyclopentane, cyclohexane and cycloheptane. Within the sub-set of cycloalkyl groups the cycloalkyl group will have from 3 to 8 carbon atoms, particular examples being C₃₋₆ cycloalkyl groups.

Examples of alkenyl groups include, but are not limited to, ethenyl (vinyl), 1-propenyl, 2-propenyl (allyl), isopropenyl, butenyl, buta-1,4-dienyl, pentenyl, and hexenyl. Within the sub-set of alkenyl groups the alkenyl group will have 2 to 8 carbon atoms, particular examples being C₂₋₆ alkenyl groups, such as C₂₋₄ alkenyl groups.

Examples of cycloalkenyl groups include, but are not limited to, cyclopropenyl, cyclobutenyl, cyclopentenyl, cyclopentadienyl and cyclohexenyl. Within the sub-set of cycloalkenyl groups the cycloalkenyl groups have from 3 to 8 carbon atoms, and particular examples are C₃₋₆ cycloalkenyl groups.

Examples of alkynyl groups include, but are not limited to, ethynyl and 2-propynyl (propargyl) groups. Within the sub-set of alkynyl groups having 2 to 8 carbon atoms, particular examples are C₂₋₆ alkynyl groups, such as C₂₋₄ alkynyl groups.

Examples of carbocyclic aryl groups include substituted and unsubstituted phenyl, naphthyl, indane and indene groups.

Examples of cycloalkylalkyl, cycloalkenylalkyl, carbocyclic aralkyl, aralkenyl and aralkenyl groups include phenethyl, benzyl, styryl, phenylethynyl, cyclohexylmethyl, cyclopentylmethyl, cyclobutylmethyl, cyclopropylmethyl and cyclopentenylmethyl groups.

The terms C₁₋₁₀ hydrocarbyl and C₁₋₈ hydrocarbyl as used herein encompass alkyl, alkenyl, alkynyl, cycloalkyl, cycloalkenyl, phenyl, benzyl and phenylethyl groups wherein the preferences for and examples of each of the aforesaid groups are as defined above. Within this definition, particular hydrocarbyl groups are alkyl, cycloalkyl, phenyl, benzyl and phenylethyl (e.g. 1-phenylethyl or 2-phenylethyl) groups, one subset of hydrocarbyl groups consisting of alkyl and cycloalkyl groups and in particular C₁₋₄ alkyl and cycloalkyl groups such as methyl, ethyl, n-propyl, isopropyl, n-butyl, isobutyl, tert-butyl, cyclopropyl and cyclobutyl.

The term C₁₋₄ hydrocarbyl as used herein encompasses alkyl, alkenyl, alkynyl, cycloalkyl and cycloalkenyl groups wherein the preferences for and examples of the aforesaid groups are as defined above. Within this definition, particular C₁₋₄ hydrocarbyl groups are alkyl and cycloalkyl groups, such as methyl, ethyl, n-propyl, isopropyl, n-butyl, isobutyl, tert-butyl, cyclopropyl and cyclobutyl.

When present, and where stated, a hydrocarbyl group can be optionally substituted by one or more substituents selected from hydroxy, oxo, alkoxy, carboxy, halogen, cyano, nitro, amino, mono- or di-C₁₋₄ hydrocarbylamino, and monocyclic or bicyclic carbocyclic and heterocyclic groups having from 3 to 12 (typically 3 to 10 and more usually 5 to 10) ring members. Preferred substituents include halogen such as fluorine. Thus, for example, the substituted hydrocarbyl group can be a partially fluorinated or perfluorinated group such as difluoromethyl or trifluoromethyl. In one embodiment preferred substituents include monocyclic carbocyclic and heterocyclic groups having 3-7 ring members.

Where stated, one or more carbon atoms of a hydrocarbyl group may optionally be replaced by O, S, SO, SO₂, NR^{c}, X¹C(X²), C(X²)X¹ or X¹C(X²)X¹ (or a sub-group thereof) wherein X¹ and X² are as hereinbefore defined, provided that at least one carbon atom of the hydrocarbyl group remains. For example, 1, 2, 3 or 4 carbon atoms of the hydrocarbyl group may be replaced by one of the atoms or groups listed, and the replacing atoms or groups may be the same or different. In general, the number of linear or backbone carbon atoms replaced will correspond to the number of linear or backbone atoms in the group replacing them. Examples of groups in which one or more carbon atom of the hydrocarbyl group have been replaced by a replacement atom or group as defined above include ethers and thioethers (C replaced by O or S), amides, esters, thioamides and thioesters (C—C replaced by X¹C(X²) or C(X²)X¹), sulphones and sulphoxides (C replaced by SO or SO₂), amines (C replaced by NR^{c}). Further examples include ureas, carbonates and carbamates (C—C—C replaced by X¹C(X²)X¹).

Where an amino group has two hydrocarbyl substituents, they may, together with the nitrogen atom to which they are attached, and optionally with another heteroatom such as nitrogen, sulphur, or oxygen, link to form a ring structure of 4 to 7 ring members.

The definition “R^{a}-R^{b}” as used herein, either with regard to substituents present on a carbocyclic or heterocyclic moiety, or with regard to other substituents present at other locations on the compounds of the formula (I), includes inter alia compounds wherein R^{a} is selected from a bond, O, CO, OC(O), SC(O), NR^{c}C(O), OC(S), SC(S), NR^{c}C(S), OC(NR^{c}), SC(NR^{c}), NR^{c}C(NR^{c}), C(O)O, C(O)S, C(O)NR^{c}, C(S)O, C(S)S, C(S)NR^{c}, C(NR^{c})O, C(NR^{c})S, C(NR^{c})NR^{c}, OC(O)O, SC(O)O, NR^{c}C(O)O, OC(S)O, SC(S)O, NR^{c}C(S)O, OC(NR^{c})O, SC(NR^{c})O, NR^{c}C(NR^{c})O, OC(O)S, SC(O)S, NR^{c}C(O)S, OC(S)S, SC(S)S, NR^{c}C(S)S, OC(NR^{c})S, SC(NR^{c})S, NR^{c}C(NR^{c})S, OC(O)NR^{c}, SC(O)NR^{c}, NR^{c}C(O)NR^{c}, OC(S)NR^{c}, SC(S)NR^{c}, NR^{c}C(S)NR^{c}, OC(NR^{c})NR^{c}, SC(NR^{c})NR^{c}, NR^{c}C(NR^{c}NR^{c}, S, SO, SO₂, NR^{c}, SO₂NR^{c} and NR^{c}SO₂ wherein R^{c} is as hereinbefore defined.

The moiety R^{b} can be hydrogen or it can be a group selected from carbocyclic and heterocyclic groups having from 3 to 12 ring members (typically 3 to 10 and more usually from 5 to 10), and a C₁₋₈ hydrocarbyl group optionally substituted as hereinbefore defined. Examples of hydrocarbyl, carbocyclic and heterocyclic groups are as set out above.

When R^{a} is O and R^{b} is a C₁₋₈ hydrocarbyl group, R^{a} and R^{b} together form a hydrocarbyloxy group. Preferred hydrocarbyloxy groups include saturated hydrocarbyloxy such as alkoxy (e.g. C₁₋₆ alkoxy, more usually C₁₋₄ alkoxy such as ethoxy and methoxy, particularly methoxy), cycloalkoxy (e.g. C₃₋₆ cycloalkoxy such as cyclopropyloxy, cyclobutyloxy, cyclopentyloxy and cyclohexyloxy) and cycloalkylalkoxy (e.g. C₃₋₆ cycloalkyl-C₁₋₂ alkoxy such as cyclopropylmethoxy).

The hydrocarbyloxy groups can be substituted by various substituents as defined herein. For example, the alkoxy groups can be substituted by halogen (e.g. as in difluoromethoxy and trifluoromethoxy), hydroxy (e.g. as in hydroxyethoxy), C₁₋₂ alkoxy (e.g. as in methoxyethoxy), hydroxy-C₁₋₂ alkyl (as in hydroxyethoxyethoxy) or a cyclic group (e.g. a cycloalkyl group or non-aromatic heterocyclic group as hereinbefore defined). Examples of alkoxy groups bearing a non-aromatic heterocyclic group as a substituent are those in which the heterocyclic group is a saturated cyclic amine such as morpholine, piperidine, pyrrolidine, piperazine, C₁₋₄-alkyl-piperazines, C₃₋₇-cycloalkyl-piperazines, tetrahydropyran or tetrahydrofuran and the alkoxy group is a C₁₋₄ alkoxy group, more typically a C₁₋₃ alkoxy group such as methoxy, ethoxy or n-propoxy.

Alkoxy groups may be substituted by, for example, a monocyclic group such as pyrrolidine, piperidine, morpholine and piperazine and N-substituted derivatives thereof such as N-benzyl, N—C₁₋₄ acyl and N—C₁₋₄ alkoxycarbonyl. Particular examples include pyrrolidinoethoxy, piperidinoethoxy and piperazinoethoxy.

When R^{a} is a bond and R^{b} is a C₁₋₈ hydrocarbyl group, examples of hydrocarbyl groups R^{a}-R^{b} are as hereinbefore defined. The hydrocarbyl groups may be saturated groups such as cycloalkyl and alkyl and particular examples of such groups include methyl, ethyl and cyclopropyl. The hydrocarbyl (e.g. alkyl) groups can be substituted by various groups and atoms as defined herein. Examples of substituted alkyl groups include alkyl groups substituted by one or more halogen atoms such as fluorine and chlorine (particular examples including bromoethyl, chloroethyl, difluoromethyl, 2,2,2-trifluoroethyl and perfluoroalkyl groups such as trifluoromethyl), or hydroxy (e.g. hydroxymethyl and hydroxyethyl), C₁₋₈ acyloxy (e.g. acetoxymethyl and benzyloxymethyl), amino and mono- and dialkylamino (e.g. aminoethyl, methylaminoethyl, dimethylaminomethyl, dimethylaminoethyl and tert-butylaminomethyl), alkoxy (e.g. C₁₋₂ alkoxy such as methoxy—as in methoxyethyl), and cyclic groups such as cycloalkyl groups, aryl groups, heteroaryl groups and non-aromatic heterocyclic groups as hereinbefore defined).

Particular examples of alkyl groups substituted by a cyclic group are those wherein the cyclic group is a saturated cyclic amine such as morpholine, piperidine, pyrrolidine, piperazine, C₁₋₄-alkyl-piperazines, C₃₋₇-cycloalkyl-piperazines, tetrahydropyran or tetrahydrofuran and the alkyl group is a C₁₋₄ alkyl group, more typically a C₁₋₃ alkyl group such as methyl, ethyl or n-propyl. Specific examples of alkyl groups substituted by a cyclic group include pyrrolidinomethyl, pyrrolidinopropyl, morpholinomethyl, morpholinoethyl, morpholinopropyl, piperidinylmethyl, piperazinomethyl and N-substituted forms thereof as defined herein.

Particular examples of alkyl groups substituted by aryl groups and heteroaryl groups include benzyl, phenethyl and pyridylmethyl groups.

When R^{a} is SO₂NR^{c}, R^{b} can be, for example, hydrogen or an optionally substituted C₁₋₈ hydrocarbyl group, or a carbocyclic or heterocyclic group. Examples of R^{a}-R^{b} where R^{a} is SO₂NR^{c} include aminosulphonyl, C₁₋₄ alkylaminosulphonyl and di-C₁₋₄ alkylaminosulphonyl groups, and sulphonamides formed from a cyclic amino group such as piperidine, morpholine, pyrrolidine, or an optionally N-substituted piperazine such as N-methyl piperazine.

Examples of groups R^{a}-R^{b} where R^{a} is SO₂ include alkylsulphonyl, heteroarylsulphonyl and arylsulphonyl groups, particularly monocyclic aryl and heteroaryl sulphonyl groups. Particular examples include methylsulphonyl, phenylsulphonyl and toluenesulphonyl.

When R^{a} is NR^{c}, R^{b} can be, for example, hydrogen or an optionally substituted C₁₋₈ hydrocarbyl group, or a carbocyclic or heterocyclic group. Examples of R^{a}-R^{b} where R^{a} is NR^{c} include amino, C₁₋₄ alkylamino (e.g. methylamino, ethylamino, propylamino, isopropylamino, tert-butylamino), di-C₄ alkylamino (e.g. dimethylamino and diethylamino) and cycloalkylamino (e.g. cyclopropylamino, cyclopentylamino and cyclohexylamino).

### Specific Embodiments of and Preferences for A, E, T, Q¹, Q², J¹, J² and R¹ to R¹⁰

### The Group “A”

In compounds of the formula (I) wherein TG is:

(1) a group:

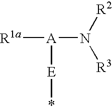

A is a saturated hydrocarbon linker group containing from 1 to 7 carbon atoms, the linker group having a maximum chain length of 5 atoms extending between R^{1a} and NR²R³ and a maximum chain length of 4 atoms extending between E and NR²R³. Within these constraints, the moieties E and R^{1a} can each be attached at any location on the group A.

The term “maximum chain length” as used herein refers to the number of atoms lying directly between the two moieties in question, and does not take into account any branching in the chain or any hydrogen atoms that may be present. For example, in the structure (A) shown below:

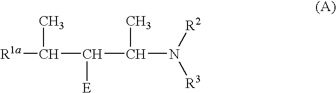

the chain length between R^{1a} and NR²R³ is 3 atoms whereas the chain length between E and NR²R³ is 2 atoms.

In general it is presently preferred that the linker group has a maximum chain length of 3 atoms (for example 1 or 2 atoms).

In one embodiment, the linker group has a chain length of 1 atom extending between R^{1a} and NR²R³.

In another embodiment, the linker group has a chain length of 2 atoms extending between R^{1a} and NR²R³.

In a further embodiment, the linker group has a chain length of 3 atoms extending between R¹ and NR²R³.

It is preferred that the linker group has a maximum chain length of 3 atoms extending between E and NR²R³.

In one particularly preferred group of compounds, the linker group has a chain length of 2 or 3 atoms extending between R^{1a} and NR²R³ and a chain length of 2 or 3 atoms extending between E and NR²R³.

One of the carbon atoms in the linker group may optionally be replaced by an oxygen or nitrogen atom.

When present, the nitrogen atom may be linked directly to the group E.

In one embodiment, the carbon atom to which the group R^{1a} is attached is replaced by an oxygen atom.

In another embodiment, R^{1a} and E are attached to the same carbon atom of the linker group, and a carbon atom in the chain extending between E and NR²R³ is replaced by an oxygen atom.

When a nitrogen atom or oxygen atom are present, it is preferred that the nitrogen or oxygen atom and the NR²R³ group are spaced apart by at least two intervening carbon atoms.

In one particular group of compounds within formula (I), the linker atom linked directly to the group E is a carbon atom and the linker group A has an all-carbon skeleton.

The carbon atoms of the linker group A may optionally bear one or more substituents selected from oxo, fluorine and hydroxy, provided that the hydroxy group is not located at a carbon atom α with respect to the NR²R³ group, and provided also that the oxo group is located at a carbon atom α with respect to the NR²R³ group. Typically, the hydroxy group, if present, is located at a position β with respect to the NR²R³ group. In general, no more than one hydroxy group will be present. Where fluorine is present, it may be present as a single fluorine substituent or may be present in a difluoromethylene or trifluoromethyl group, for example. In one embodiment, a fluorine atom is located at a position p with respect to the NR²R³ group.

It will be appreciated that that when an oxo group is present at the carbon atom adjacent the NR²R³ group, the compound of the formula (I) will be an amide.

In one embodiment of the invention, no fluorine atoms are present in the linker group A.

In another embodiment of the invention, no hydroxy groups are present in the linker group A.

In a further embodiment, no oxo group is present in the linker group A.

In one group of compounds of the formula (I) neither hydroxy groups nor fluorine atoms are present in the linker group A, e.g. the linker group A is unsubstituted.

Preferably, when a carbon atom in the linker group A is replaced by a nitrogen atom, the group A bears no more than one hydroxy substituent and more preferably bears no hydroxy substituents.

When there is a chain length of four atoms between E and NR²R³, it is preferred that the linker group A contains no nitrogen atoms and more preferably has an all carbon skeleton.

The linker group A can have a branched configuration at the carbon atom attached to the NR²R³ group. For example, the carbon atom attached to the NR²R³ group can be attached to a pair of gem-dimethyl groups.

In one particular group of compounds of the formula (I), the portion R^{1a}-A-NR²R³ of the compound is represented by the formula R^{1a}-(G)ₖ-(CH₂)ₘ—W—O_{b}—(CH₂)ₙ—(CR⁶R⁷)ₚ—NR²R³ wherein G is NH, NMe or O; W is attached to the group E and is selected from (CH₂)ⱼ—CR²⁰, (CH₂)ⱼ—N and (NH)ⱼ—CH; b is 0 or 1, j is 0 or 1, k is 0 or 1, m is 0 or 1, n is 0, 1, 2, or 3 and p is 0 or 1; the sum of b and k is 0 or 1; the sum of j, k, m, n and p does not exceed 4; R⁶ and R⁷ are the same or different and are selected from methyl and ethyl, or CR⁶R⁷ forms a cyclopropyl group; and R²⁰ is selected from hydrogen, methyl, hydroxy and fluorine;

In another sub-group of compounds of the formula (I), the portion R^{1a}-A-NR²R³ of the compound is represented by the formula R^{1a}-(G)ₖ-(CH₂)ₘ—X^{x}—(CH₂)ₙ—(CR⁶R⁷)ₚ—NR²R³ wherein G is NH, NMe or O; X^{x} is attached to the group E and is selected from (CH₂)ⱼ—CH, (CH₂)ⱼ—N and (NH)ⱼ—CH; j is 0 or 1, k is 0 or 1, m is 0 or 1, n is 0, 1, 2, or 3 and p is 0 or 1, and the sum of j, k, m, n and p does not exceed 4; and R⁶ and R⁷ are the same or different and are selected from methyl and ethyl, or CR⁶R⁷ forms a cyclopropyl group.

A particular group CR⁶R⁷ is C(CH₃)₂.

Preferably X^{x} is (CH₂)ⱼ—CH.

Particular configurations where the portion R^{1a}-A-NR²R³ of the compound is represented by the formula R¹-(G)ₖ-(CH₂)ₘ—X^{x}—(CH₂)ₙ—(CR⁶R⁷)ₚ—NR² are those wherein:
k is 0, m is 0 or 1, n is 0, 1, 2 or 3 and p is 0.
k is 0, m is 0 or 1, n is 0, 1 or 2 and p is 1.
X^{x} is (CH₂)ⱼ—CH, k is 1, m is 0, n is 0, 1, 2 or 3 and p is 0.
X^{x} is (CH₂)ⱼ—CH, k is 1, m is 0, n is 0, 1 or 2 and p is 1.
X^{x} is (CH₂)ⱼ—CH, G is O, k is 1, m is 0, n is 0, 1, 2 or 3 and p is 0.

Particular configurations wherein the portion R^{1a}-A-NR²R³ of the compound is represented by the formula R¹-(G)ₖ-(CH₂)ₘ—W—O_{b}—(CH₂)ₙ—(CR⁶R⁷)ₚ—NR²R³ are those wherein:
k is 0, m is 0, W is (CH₂)ⱼ—CR²⁰, j is 0, R²⁰ is hydrogen, b is 1, n is 2 and p is 0.
k is 0, m is 0, W is (CH₂)ⱼ—CR²⁰, j is 0, R²⁰ is hydroxy, b is 0, n is 1 and p is 0.
k is 0, m is 0, W is (CH₂)ⱼ—CR²⁰, j is 0, R²⁰ is methyl, b is 0, n is 1 and p is 0.
k is 0, m is 0, W is (CH₂)ⱼ—CR²⁰, j is 0, R²⁰ is fluorine, b is 0, n is 1 and p is 0.

In one preferred configuration, the portion R^{1a}-A-NR²R³ of the compound is represented by the formula R^{1a}—X^{x}—(CH₂)ₙ—NR²R³ wherein X^{x} is attached to the group E and is a group CH, and n is 2.

Particular examples of the linker group A, together with their points of attachment to the groups R^{1a}, E and NR²R³, are shown in Table 1 below.

TABLE 1

| | |
|---|---|
| 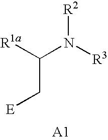 | 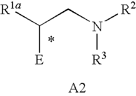 |
| 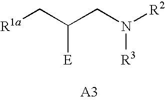 | 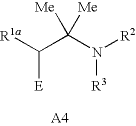 |
| 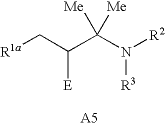 | 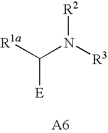 |
| 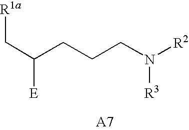 | 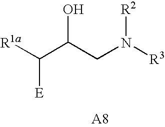 |
| 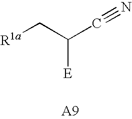 | 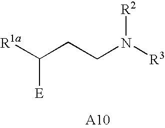 |
| 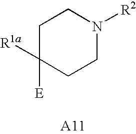 | 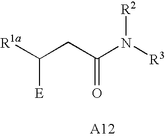 |
| 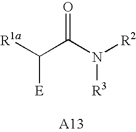 | 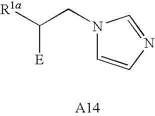 |
| 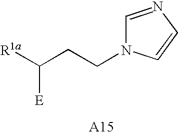 | 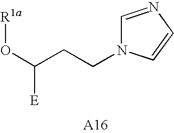 |
| 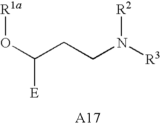 | 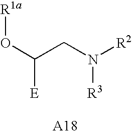 |
| 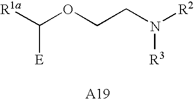 | 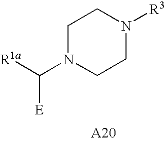 |
| 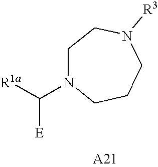 | 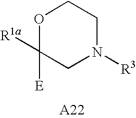 |
| 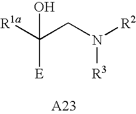 | 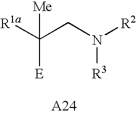 |
| 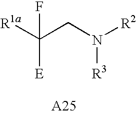 | |

Currently preferred groups include A1, A2, A3, A6, A10, A11, A22 and A23.

One particular set of groups includes A1, A2, A3, A10 and A11.

A further particular set of groups includes A2 and A11.

Another particular set of groups includes A6, A22 and A23.

A further set of groups includes A1, A2 and A3.

In group A2, the asterisk designates a chiral centre. Compounds having the R configuration at this chiral centre represent one preferred sub-group of compounds of the invention.

### Q¹ and Q²

In compounds of the formula (I) wherein TG is:

(2) a group:

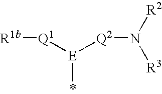

Q¹ is a bond or a saturated hydrocarbon linker group containing from 1 to 3 carbon atoms, wherein one of the carbon atoms in the linker group may optionally be replaced by an oxygen or nitrogen atom, or an adjacent pair of carbon atoms may be replaced by CONR^{q} or NR^{q}CO where R^{q} is hydrogen, C₁₋₄ alkyl or cyclopropyl, or R^{q} is a C₁₋₄ alkylene chain that links to R^{1b} or to another carbon atom of Q¹ to form a cyclic moiety; and wherein the carbon atoms of the linker group Q¹ may optionally bear one or more substituents selected from fluorine and hydroxy.

Q² is a bond or a saturated hydrocarbon linker group containing from 1 to 3 carbon atoms, wherein one of the carbon atoms in the linker group may optionally be replaced by an oxygen or nitrogen atom; and wherein the carbon atoms of the linker group may optionally bear one or more substituents selected from fluorine and hydroxy, provided that the hydroxy group when present is not located at a carbon atom α with respect to the G group.

In one embodiment, Q¹ and Q² are the same or different and are each a bond or a saturated hydrocarbon linker group containing from 1 to 3 carbon atoms, wherein one of the carbon atoms in the linker group may optionally be replaced by an oxygen or nitrogen atom; and wherein the carbon atoms of the or each linker group Q¹ and Q² may optionally bear one or more substituents selected from fluorine and hydroxy, provided that the hydroxy group when present is not located at a carbon atom α with respect to the NR²R³ group.

In one group of compounds of the invention, at least one of Q¹ and Q² represents a bond. Within this group of compounds, one sub-group consists of compounds in which both of Q¹ and Q² represent a bond. In another sub-group, one of Q¹ and Q² represents a bond, and the other represents a saturated hydrocarbon linker group containing from 1 to 3 carbon atoms, wherein one of the carbon atoms in the linker group may optionally be replaced by an oxygen or nitrogen atom.

When Q¹ and/or Q² are saturated hydrocarbon groups, the hydrocarbon groups are typically alkylene groups such as (CH₂)ₙ where n is 1, 2 or 3, one particular example being CH₂. One of the carbon atoms in the alkylene group Q¹ may optionally be replaced by, for example, an oxygen atom, and an example of such a group is CH₂—O—CH₂.

The carbon atoms of the linker groups Q¹ and Q² may optionally bear one or more substituents selected from oxo, fluorine and hydroxy, provided that the hydroxy group is not located at a carbon atom α with respect to the NR²R³ group, and provided also that the oxo group is located at a carbon atom α with respect to the NR²R³ group. Typically, the hydroxy group, if present, is located at a position β with respect to NR²R³ group. In general, no more than one hydroxy group will be present. Where fluorine atoms are present, they may be present in a difluoromethylene or trifluoromethyl group, for example.

In one sub-group of compounds, Q¹ is a saturated hydrocarbon linker group containing from 1 to 3 carbon atoms, wherein an adjacent pair of carbon atoms is replaced by CONR^{q} or NR^{q}CO where R^{q} is hydrogen, C₁₋₄ alkyl or cyclopropyl, or R^{q} is a C₁₋₄ alkylene chain that links to R^{1b} or to another carbon atom of Q¹ to form a cyclic moiety. In one preferred embodiment, R^{q} is hydrogen. In another embodiment, R^{q} is C₁₋₄ alkyl or cyclopropyl, preferably methyl. In a further embodiment, R^{q} is a C₁₋₄ alkylene chain that links to R^{1b} or to another carbon atom of Q¹ to form a cyclic moiety.

Examples of linker groups Q¹ containing CONR^{q} or NR^{q}CO are the groups CH₂NHCO and CH₂N(Me)CO where the carbonyl group is attached to E.

Examples of linker groups Q¹ containing CONR^{q} or NR^{q}CO, where R^{q} is a C₁₋₄ alkylene chain that links to another carbon atom of Q¹ to form a cyclic moiety, are groups represented by the formula:

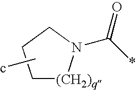

where * represents the point of attachment to the moiety E, q″ is 0, 1 or 2, and the point of attachment to R^{1b} is indicated by the letter “c”.

Examples of linker groups Q¹ containing CONR^{q} or NR^{q}CO, where R^{q} is a C₁₋₄ alkylene chain that links to R^{1b} to form a cyclic moiety, are groups represented by the formula:

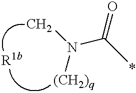

where q is as defined herein and R^{1b} is an aryl or heteroaryl group. Particular examples of moieties R^{1b}-Q¹ of this type include 1,2,3,4-tetrahydroisoquinolin-2-ylcarbonyl.

It will be appreciated that that when an oxo group is present at the carbon atom adjacent an NR²R³ group, the compound of the formula (I) will be an amide.

In one embodiment of the invention, no fluorine atoms are present in the linker groups Q¹ and/or Q².

In another embodiment of the invention, no hydroxy groups are present in the linker groups Q¹ and/or Q².

In a further embodiment, no oxo group is present in the linker groups Q¹ and/or Q².

In one group of compounds of the formula (I) neither hydroxy groups nor fluorine atoms are present in the linker groups Q¹ and/or Q², e.g. the linker groups Q¹ and/or Q² are unsubstituted.

In another group of compounds of the invention, the linker group Q² can have a branched configuration at the carbon atom attached to the NR²R³ group, when present. For example, the carbon atom attached to the NR²R³ group can be attached to a pair of gem-dimethyl groups.

Q¹ and Q² may be attached to the same atom of group E, or to different atoms. In one embodiment, Q¹ and Q² are attached to the same atom (i.e. a carbon atom) of group E.

### R^{1a}

The group R^{1a} is an aryl or heteroaryl group and may be selected from the list of such groups set out in the section headed General Preferences and Definitions.

R^{1a} can be monocyclic or bicyclic and, in one preferred embodiment, is monocyclic. Particular examples of monocyclic aryl and heteroaryl groups are six membered aryl and heteroaryl groups containing up to 2 nitrogen ring members, and five membered heteroaryl groups containing up to 3 heteroatom ring members selected from O, S and N.

Examples of such groups include phenyl, naphthyl, thienyl, furan, pyrimidine and pyridine, with phenyl being presently preferred.

The group R^{1a} can be unsubstituted or substituted by up to 5 substituents, and examples of substituents are those listed in group R¹⁰ above.

Particular substituents include hydroxy; C₁₋₄ acyloxy; fluorine; chlorine; bromine; trifluoromethyl; cyano; CONH₂; nitro; C₁₋₄ hydrocarbyloxy and C₁₋₄ hydrocarbyl each optionally substituted by C₁₋₂ alkoxy, carboxy or hydroxy; C₁₋₄ acylamino; benzoylamino; pyrrolidinocarbonyl; piperidinocarbonyl; morpholinocarbonyl; piperazinocarbonyl; five and six membered heteroaryl and heteroaryloxy groups containing one or two heteroatoms selected from N, O and S; phenyl; phenyl-C₁₋₄ alkyl; phenyl-C₁₋₄ alkoxy; heteroaryl-C₁₋₄ alkyl; heteroaryl-C₁₋₄ alkoxy and phenoxy, wherein the heteroaryl, heteroaryloxy, phenyl, phenyl-C₁₋₄ alkyl, phenyl-C₁₋₄ alkoxy, heteroaryl-C₁₋₄ alkyl, heteroaryl-C₁₋₄ alkoxy and phenoxy groups are each optionally substituted with 1, 2 or 3 substituents selected from C₁₋₂ acyloxy, fluorine, chlorine, bromine, trifluoromethyl, cyano, CONH₂, C₁₋₂ hydrocarbyloxy and C₁₋₂ hydrocarbyl each optionally substituted by methoxy or hydroxy.

Preferred substituents include hydroxy; C₁₋₄ acyloxy; fluorine; chlorine; bromine; trifluoromethyl; trifluoromethoxy; difluoromethoxy; cyano; C₁₋₄ hydrocarbyloxy and C₁₋₄ hydrocarbyl each optionally substituted by C₁₋₂ alkoxy or hydroxy; C₁₋₄ acylamino; benzoylamino; pyrrolidinocarbonyl; piperidinocarbonyl; morpholinocarbonyl; piperazinocarbonyl; five and six membered heteroaryl groups containing one or two heteroatoms selected from N, O and S, the heteroaryl groups being optionally substituted by one or more C₁₋₄ alkyl substituents; phenyl; pyridyl; and phenoxy wherein the phenyl, pyridyl and phenoxy groups are each optionally substituted with 1, 2 or 3 substituents selected from C₁₋₂ acyloxy, fluorine, chlorine, bromine, trifluoromethyl, cyano, C₁₋₂ hydrocarbyloxy and C₁₋₂ hydrocarbyl each optionally substituted by methoxy or hydroxy.

In one embodiment, the group R^{1a} is unsubstituted or substituted by up to 5 substituents selected from hydroxy; C₁₋₄ acyloxy; fluorine; chlorine; bromine; trifluoromethyl; trifluoromethoxy; difluoromethoxy; phenyl; thienyl; furanyl; phenoxy, benzyloxy; cyano; C₃₋₄ cycloalkyl, and C₁₋₄ alkoxy and C₁₋₄ hydrocarbyl each optionally substituted by C₁₋₂ alkoxy or hydroxy.

Although up to 5 substituents may be present, more typically there are 0, 1, 2, 3 or 4 substituents, preferably 0, 1, 2 or 3, and more preferably 0, 1 or 2.

In a particular sub-group of compounds, the group R^{1a} (e.g. wherein R^{1a} is a substituted phenyl group) can have one or two substituents selected from fluorine, chlorine, cyano, methyl, ethyl, isopropyl, cyclopropyl, tert-butyl; trifluoromethyl, methoxy, trifluoromethoxy; difluoromethoxy; phenyl; phenoxy and benzyloxy.

When R^{1a} is a phenyl group, particular examples of substituent combinations include mono-chlorophenyl, dichlorophenyl, hydroxyphenyl, fluoro-chlorophenyl, cyanophenyl, methoxyphenyl, methoxy-chlorophenyl, fluorophenyl and difluorophenyl.

When R^{1a} is a six membered aryl (e.g. phenyl) or heteroaryl group, a substituent may advantageously be present at the para position on the six-membered ring. Where a substituent is present at the para position, it is preferably larger in size than a fluorine atom.

In one sub-group of compounds, the group R^{1a} is a phenyl group having a substituent at the para position selected from fluorine, chlorine, trifluoromethyl, trifluoromethoxy, difluoromethoxy, benzyloxy, methyl, methoxy and tert-butyl.

In another sub-group of compounds, the group R^{1a} is a phenyl group having a substituent at the ortho position selected from fluorine, chlorine, trifluoromethyl, trifluoromethoxy, difluoromethoxy, methyl and methoxy, and optionally a second substituent at the meta or para position selected from fluorine, chlorine, trifluoromethyl, trifluoromethoxy, difluoromethoxy, methyl and methoxy.

Particular examples of the group R^{1a} are shown in Table 2 below, the point of attachment to A or Q¹ (or E when Q¹ is a bond) being indicated by means of an asterisk.

TABLE 2

| | |
|---|---|
| 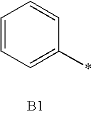 | 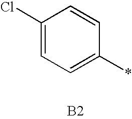 |
| 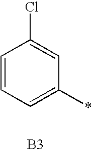 | 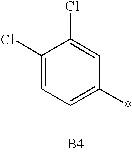 |
| 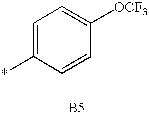 | 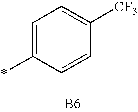 |
| 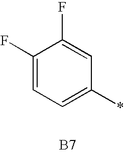 | 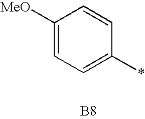 |
| 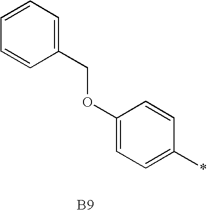 | 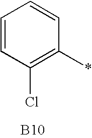 |
| 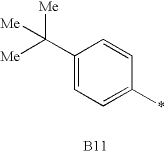 | 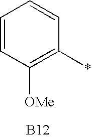 |
| 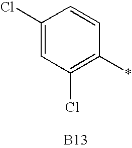 | 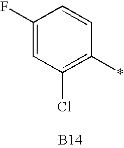 |
| 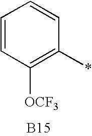 | 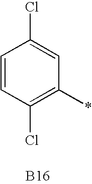 |
| 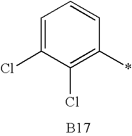 | 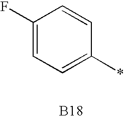 |
| 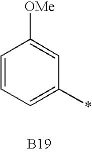 | 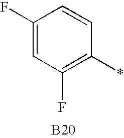 |
| 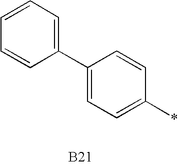 | 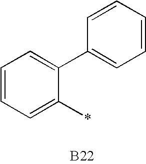 |
| 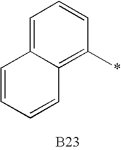 | 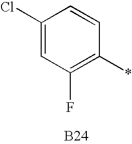 |
| 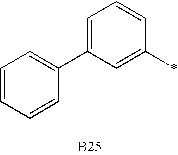 | 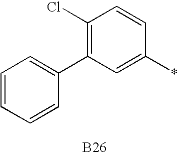 |
| 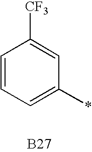 | 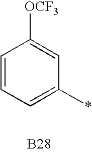 |
| 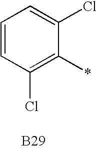 | |

One set of preferred groups includes groups B2, B4, B5, B10, B11, B13, B14, B15, B16, B17, B18, B19 and B19.

One particularly preferred group is B2.

### R^{1b}

The group R^{1b} is hydrogen or a group R^{1a} wherein is as defined above and elsewhere herein.

In one sub-group of compounds, R^{1b} is hydrogen.

In another sub-group of compounds, R^{1b} is an aryl or heteroaryl group R^{1a}.

### R² and R³

In one group of compounds of the formula (I), R² and R³ can be independently selected from hydrogen; C₁₋₄ hydrocarbyl and C₁₋₄ acyl wherein the hydrocarbyl and acyl groups are optionally substituted by one or more substituents selected from fluorine, hydroxy, cyano, amino, methylamino, dimethylamino, methoxy and a monocyclic or bicyclic aryl or heteroaryl group.

Within this group of compounds are the compounds wherein R² and R³ are independently selected from hydrogen; C₁₋₄ hydrocarbyl and C₁₋₄ acyl wherein the hydrocarbyl and acyl groups are each optionally substituted by a monocyclic or bicyclic aryl or heteroaryl group.

Also within this group of compounds is the sub-group of compounds of the invention wherein R² and R³ are independently selected from hydrogen, C₁₋₄ hydrocarbyl and C₁₋₄ acyl.

In another sub-group of compounds, R² and R³ are independently selected from hydrogen, C₁₋₄ hydrocarbyl and C₁₋₄ acyl wherein the hydrocarbyl and acyl moieties are optionally substituted by one or more substituents selected from fluorine, hydroxy, amino, methylamino, dimethylamino and methoxy.

In each of the foregoing groups and sub-groups of compounds, the hydrocarbyl group, whether substituted or unsubstituted, forming part of NR²R³ typically is an alkyl group, more usually a C₁, C₂ or C₃ alkyl group, for example a methyl group.

When the hydrocarbyl moiety is substituted by a hydroxy, amino, methylamino, dimethylamino or methoxy group, typically there are at least two carbon atoms between the substituent and the nitrogen atom of the group NR²R³. Particular examples of substituted hydrocarbyl groups are hydroxyethyl and hydroxypropyl.

In another group of compounds of the invention, R² and R³ are independently selected from hydrogen, C₁₋₄ hydrocarbyl and C₁₋₄ acyl.

In a particular sub-group of compounds, R² and R³ are independently selected from hydrogen and methyl and hence NR²R³ can be an amino, methylamino or dimethylamino group.

In one embodiment, NR²R³ is an amino group. In another particular embodiment, NR²R³ is a methylamino group.

In an alternative embodiment, the C₁₋₄ hydrocarbyl group can be a cyclopropyl, cyclopropylmethyl or cyclobutyl group.

In another group of compounds, R² and R³ together with the nitrogen atom to which they are attached form a cyclic group selected from an imidazole group and a saturated monocyclic heterocyclic group having 4-7 ring members and optionally containing a second heteroatom ring member selected from O and N.

In a further group of compounds, R² and R³ together with the nitrogen atom to which they are attached form a saturated monocyclic heterocyclic group having 4-7 ring members and optionally containing a second heteroatom ring member selected from O and N.

The saturated monocyclic heterocyclic group can be unsubstituted or substituted by one or more substituents R¹⁰ as defined above in the General Preferences and Definitions section of this application. Typically, however, any substituents on the heterocyclic group will be relatively small substituents such as C₁₋₄ hydrocarbyl (e.g. methyl, ethyl, n-propyl, i-propyl, cyclopropyl, n-butyl, sec-butyl and tert-butyl), fluorine, chlorine, hydroxy, amino, methylamino, ethylamino and dimethylamino. Particular substituents are methyl groups.

The saturated monocyclic ring can be an azacycloalkyl group such as an azetidine, pyrrolidine, piperidine or azepane ring, and such rings are typically unsubstituted. Alternatively, the saturated monocyclic ring can contain an additional heteroatom selected from O and N, and examples of such groups include morpholine and piperazine. Where an additional N atom is present in the ring, this can form part of an NH group or an N—C₁₋₄alkyl group such as an N-methyl, N-ethyl, N-propyl or N-isopropyl group.

Where NR²R³ forms an imidazole group, the imidazole group can be unsubstituted or substituted, for example by one or more relatively small substituents such as C₁₋₄ hydrocarbyl (e.g. methyl, ethyl, propyl, cyclopropyl and butyl), fluorine, chlorine, hydroxy, amino, methylamino, ethylamino and dimethylamino. Particular substituents are methyl groups.

In formula (I), when TG is a group (1), one of R² and R³ together with the nitrogen atom to which they are attached and one or more atoms from the linker group A can form a saturated monocyclic heterocyclic group having 4-7 ring members and optionally containing a second heteroatom ring member selected from O and N.

Examples of such compounds include compounds wherein NR²R³ and A form a unit of the formula:

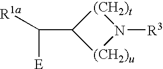

where t and u are each 0, 1, 2 or 3 provided that the sum of t and u falls within the range of 2 to 4.

Further examples of such compounds include compounds wherein NR²R³ and A form a cyclic group of the formula:

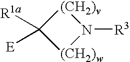

where v and w are each 0, 1, 2 or 3 provided that the sum of v and w falls within the range of 2 to 5. Particular examples of cyclic compounds are those in which v and w are both 2.

Further examples of such compounds include compounds wherein NR²R³ and A form a cyclic group of the formula:

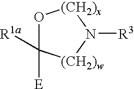

where x and w are each 0, 1, 2 or 3 provided that the sum of x and w falls within the range of 2 to 4. Particular examples of cyclic compounds are those in which x is 2 and w is 1.

In another embodiment of formula (I) wherein TG is a group (2), NR²R³ and a carbon atom of linker group Q² to which it is attached from a cyano group.

In a further embodiment of formula (I) wherein TG is a group (2), NR²R³ is as hereinbefore defined except that NR²R³ and a carbon atom of linker group Q² to which it is attached may not form a cyano group.

In a further embodiment of formula (I) wherein TG is a group (2), one of R² and R³ together with the nitrogen atom to which they are attached and one or more atoms from the linker group Q² form a saturated monocyclic heterocyclic group having 4-7 ring members and optionally containing a second heteroatom ring member selected from O and N.

### The Group “E”

In formula (I), E is a monocyclic or bicyclic carbocyclic or heterocyclic group and can be selected from the groups set out above in the section headed General Preferences and Definitions.

The carbocyclic or heterocyclic group E can be aromatic or non-aromatic.

In one embodiment, the carbocyclic or heterocyclic group E is non-aromatic.

In another embodiment, the carbocyclic or heterocyclic group E is aromatic.

When E is an aromatic group, i.e. an aryl or heteroaryl group, the group can be selected from the examples of such groups set out in the General Preferences and Definitions section above.

In formula (I), wherein TG is a group (2)

Preferred groups E are monocyclic and bicyclic aryl and heteroaryl groups and, in particular, groups containing a five or six membered aromatic or heteroaromatic ring such as a phenyl, pyridine, pyrazole, pyrazine, pyridazine or pyrimidine ring, more particularly a phenyl, pyridine, pyrazole, pyrazine or pyrimidine ring, more preferably a pyridine, pyrazole or phenyl ring, and most preferably a phenyl ring.

Examples of bicyclic groups include benzo-fused and pyrido-fused groups wherein the group A and the cyclic group X are both attached to the benzo- or pyrido-moiety.

In one embodiment, E is a monocyclic group.

The monocyclic carbocyclic or heterocyclic group typically contains 5 or 6 ring members and the heterocyclic group typically contains up to 3 heteroatoms selected from O, N and S.

Particular examples of monocyclic groups include monocyclic aryl and heteroaryl groups such as phenyl, thiophene, furan, pyrazole, pyrimidine, pyrazine and pyridine, phenyl being presently preferred.

One subset of monocyclic aryl and heteroaryl groups comprises phenyl, pyrazole, thiophene, furan, pyrimidine and pyridine.

Examples of non-aromatic monocyclic groups are as set out in the General Preferences and Definitions section above.

Particular examples of groups include cycloalkanes such as cyclohexane and cyclopentane, and nitrogen-containing rings such as piperidine, pyrrolidine, piperidine, piperazine and piperazone.

One particular non-aromatic monocyclic group is a piperidine group and more particularly a piperidine group wherein the nitrogen atom of the piperidine ring is attached to the bicyclic group.

Another particular group is a piperazine group wherein one nitrogen atom of the piperidine ring is attached to the bicyclic group and the other nitrogen atom of the piperidine ring is attached to the group A.

In compounds of the formula (I) wherein TG is group (1) it is preferred that the group A and the cyclic group X are not attached to adjacent ring members of the group E. For example, the cyclic group X can be attached to the group E in a meta or para relative orientation. Examples of such groups E include 1,4-phenylene, 1,3-phenylene, 2,5-pyridylene and 2,4-pyridylene, 1,4-piperazinyl, and 1,4-piperazonyl. Further examples include 1,3-disubstituted five membered rings.

The groups E can be unsubstituted or can have up to 4 substituents R⁸ which may be selected from the group R¹⁰ as hereinbefore defined. More typically however, the substituents R⁸ are selected from hydroxy; oxo (when E is non-aromatic); halogen (e.g. chlorine and bromine); trifluoromethyl; cyano; C₁₋₄ hydrocarbyloxy optionally substituted by C₁₋₂ alkoxy or hydroxy; C₁₋₄ hydrocarbyl optionally substituted by C₁₋₂ alkoxy or hydroxy; and phenyl optionally substituted by halogen (e.g. chlorine and bromine), trifluoromethyl, cyano, methyl or methoxy.

Preferably there are 0-3 substituents, more preferably 0-2 substituents, for example 0 or 1 substituent. In one embodiment, the group E is unsubstituted.

E may be other than:
a substituted pyridone group;
a substituted thiazole group;
a substituted or unsubstituted pyrazole or pyrazolone group;
a substituted or unsubstituted bicyclic fused pyrazole group;
a phenyl ring fused to a thiophene ring or a six membered nitrogen-containing heteroaryl ring fused to a thiophene ring;
a substituted or unsubstituted piperazine group;

In compounds of the formula (I) wherein the moiety TG is a group (1), the group E can be an aryl or heteroaryl group having five or six members and containing up to three heteroatoms selected from O, N and S, the group E being represented by the formula:

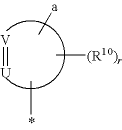

where * denotes the point of attachment to the cyclic group X, and “a” denotes the attachment of the group A;
r is 0, 1 or 2;
U is selected from N and CR^{12a}; and
V is selected from N and CR^{12b}; where R^{12a} and R^{12b} are the same or different and each is hydrogen or a substituent containing up to ten atoms selected from C, N, O, F, Cl and S provided that the total number of non-hydrogen atoms present in R^{12a} and R^{12b} together does not exceed ten;
or R^{12a} and R^{12b} together with the carbon atoms to which they are attached form an unsubstituted five or six membered saturated or unsaturated ring containing up to two heteroatoms selected from O and N; and
R¹⁰ is as hereinbefore defined.

In one preferred group of compounds, E is a group:

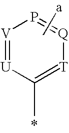

where * denotes the point of attachment to the cyclic group X, and “a” denotes the attachment of the group A;
P, Q and T are the same or different and are selected from N, CH and NCR¹⁰, provided that the group A is attached to a carbon atom; and U, V and R¹⁰ are as hereinbefore defined.

In another preferred group of compounds, E is a group:

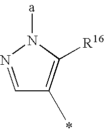

wherein R¹⁶ is hydrogen or a group R¹⁰, R^{12a} or R^{12b} as defined herein.

Examples of R^{12a} and R^{12b} include hydrogen and substituent groups R¹⁰ as hereinbefore defined having no more than ten non-hydrogen atoms. Particular examples of R^{12a} and R^{12b} include methyl, ethyl, propyl, isopropyl, cyclopropyl, cyclobutyl, cyclopentyl, phenyl, fluorine, chlorine, methoxy, trifluoromethyl, hydroxymethyl, hydroxyethyl, methoxymethyl, difluoromethoxy, trifluoromethoxy, 2,2,2-trifluoroethyl, cyano, amino, methylamino, dimethylamino, CONH₂, CO₂Et, CO₂H, acetamido, azetidinyl, pyrrolidino, piperidine, piperazino, morpholino, methylsulphonyl, aminosulphonyl, mesylamino and trifluoroacetamido.

Preferably, when U is CR^{12a} and/or V is CR^{12b} the atoms or groups in R^{12a} and R^{12b} that are directly attached to the carbon atom ring members C are selected from H, O (e.g. as in methoxy), NH (e.g. as in amino and methylamino) and CH₂ (e.g. as in methyl and ethyl).

I formula (I), when TG is a group (1), particular examples of the group E, together with their points of attachment to the group A (^{a}) and the ring X (*) are shown in Table 3 below.

TABLE 3

| | |
|---|---|
| 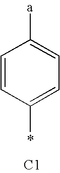 | 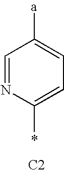 |
| 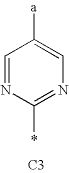 | 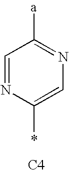 |
| 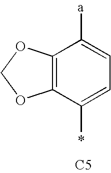 | 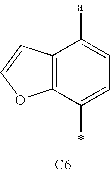 |
| 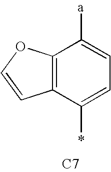 | 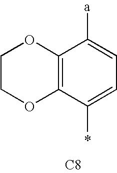 |
| 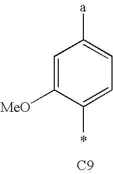 | 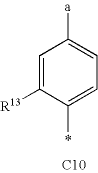 |
| 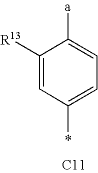 | 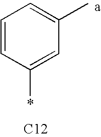 |
| 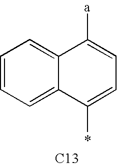 | 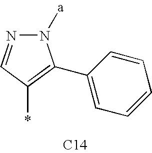 |

In the table, the substituent group R¹³ is selected from methyl, chlorine, fluorine and trifluoromethyl.

The following optional exclusions may apply to the definition of E in any of formulae (I), (II), (IIa), (IIb), (IIc), (III), (IV), (V), (VI), (VII), (VIII) and (IX) and any sub-groups or sub-definitions thereof as defined herein:
E may be other than a phenyl group having a sulphur atom attached to the position para with respect to the group X.
E may be other than a substituted or unsubstituted benzimidazole, benzoxazole or benzthiazole group.

In formula (I), wherein the moiety TG is a group (2), the moieties Q¹ and Q² can be attached to the same carbon atom in the group E or they can be attached to separate atoms. It will be appreciated that when the group E is aromatic, Q¹ and Q² cannot be attached to the same carbon atom in the group E but may be, for example, attached to adjacent carbon atoms.

In one embodiment, E is non-aromatic and Q¹ and Q² are attached to the same carbon atom in the group E.

In another embodiment, Q¹ and Q² are attached to different atoms in the group E.

It is preferred that the group Q² and the bicyclic group are attached to the group E in a meta or para relative orientation; i.e. Q² and the bicyclic group are not attached to adjacent ring members of the group E. Examples of groups such groups E include 1,4-phenylene, 1,3-phenylene, 2,5-pyridylene and 2,4-pyridylene, 1,4-piperidinyl, 1,4-piperindonyl, 1,4-piperazinyl, and 1,4-piperazonyl.

The groups E can be unsubstituted or can have up to 4 substituents R¹¹ which may be selected from the group R¹⁰ as hereinbefore defined. More typically however, the substituents R¹¹ are selected from hydroxy; oxo (when E is non-aromatic); halogen (e.g. chlorine and bromine); trifluoromethyl; cyano; C₁₋₄ hydrocarbyloxy optionally substituted by C₁₋₂ alkoxy or hydroxy; and C₁₋₄ hydrocarbyl optionally substituted by C₁₋₂ alkoxy or hydroxy.

Typically, there are 0-3 substituents, more usually 0-2 substituents, for example 0 or 1 substituent. In one embodiment, the group E is unsubstituted.

In one particular group of compounds of the invention, E is a group:

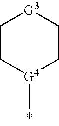

where G³ is selected from C, CH, CH₂, N and NH; and G⁴ is selected from N and CH.

Particular examples of the group E, together with their points of attachment to the groups Q¹ and Q² (a) and the bicyclic group (*) are shown in Table 4 below.

TABLE 4

| | |
|---|---|
| 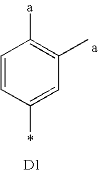 | 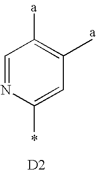 |
| 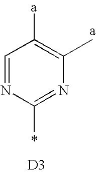 | 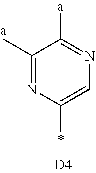 |
| 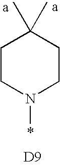 | 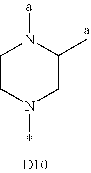 |
| 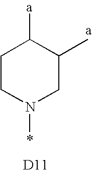 | 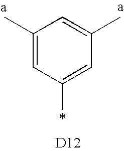 |

One preferred group E is group D9.

### The Group X

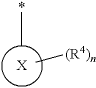

The cyclic group X is a bicyclic heterocyclic group having 8 to 12 ring members of which up to 5 are heteroatoms selected from O, N and S.

Examples of bicyclic heterocyclic groups are as set out above in the General Preferences and Definitions section.

Typically, the cyclic group X has 8 to 10 ring members for example 9 or 10 ring members.

In one embodiment, the cyclic group X is an optionally substituted bicyclic heteroaryl group.

Examples of bicyclic heteroaryl groups include pyridine or pyrimidine rings fused to a 5- or 6-membered carbocyclic or heterocyclic aromatic ring. One particular examples of such a group is the thieno[3,2-d]pyrimidine group.

In one embodiment, the cyclic group X may take the form:

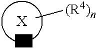

where G⁵ is a hydrogen bond acceptor atom or group.

The term “hydrogen bond acceptor” is a well established term and refers to a group capable of forming a hydrogen bond with a hydrogen atom in the same or an adjacent molecule; see for example “Advanced Organic Chemistry” by Jerry March, 4^{th} edition, pages 75-79 and references therein. In the present context, hydrogen bond acceptors include nitrogen, oxygen and sulphur atoms; and groups containing nitrogen, oxygen and sulphur atoms.

Particular examples of hydrogen bond acceptors are the groups set out in Table 5 below. The asterisk marks the point of attachment to the group E.

TABLE 5

| | |
|---|---|
| 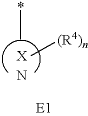 | 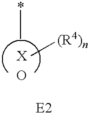 |
| 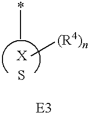 | 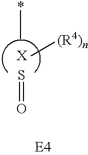 |
| 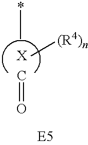 | 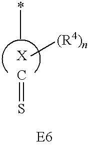 |
| 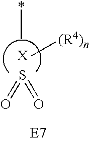 | 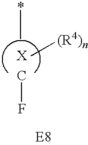 |

A cyclic group X may contain one hydrogen bond acceptor, or more than one (e.g. two or three) hydrogen bond acceptor moieties.

The cyclic group X may contain a hydrogen bond donor group adjacent the group G⁵ and hence the cyclic group X may take the form:

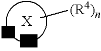

where G⁵ is a hydrogen bond acceptor atom or group and D is a hydrogen bond donor group.

The hydrogen bond donor group can be, for example, NH, C—NH₂, C—NH, C—OH, C—SH, or C—H.

### n & R⁴

Excluding from consideration any atoms or groups that may form part of the hydrogen acceptor G⁵ where present, the cyclic group X may be an unsubstituted ring system (n=0) or a substituted ring system (n=1, 2, 3 or 4).

In formula (I), R⁴ is independently selected from oxo; halogen; C₁₋₆ hydrocarbyl optionally substituted by halogen, hydroxy or C₁₋₂ alkoxy; cyano; C₁₋₆ hydrocarbyloxy optionally substituted by halogen, hydroxy or C₁₋₂ alkoxy; CONH₂; CONHR⁹; CF₃; NH₂; NHCOR⁹; NHCONHR⁹; and NHR⁹.

More typically, R⁴ is selected from oxo, amino, NHCOR⁹; NHR⁹; halogen, C₁₋₅ saturated hydrocarbyl, cyano and CF₃. Preferred values for R⁴ include oxo and methyl.

Preferably n is 0, 1 or 2.

In one embodiment, n is 0.

In another embodiment, n is 1 or 2.

Where R⁴ is CONHR⁹, NHCOR⁹; NHCONHR⁹; or NHR⁹; R⁹ is a group R^{9a} or (CH₂)R^{9a}, wherein R^{9a} is a monocyclic or bicyclic group which may be carbocyclic or heterocyclic.

Examples of carbocyclic and heterocyclic groups are set out above in the General Preferences and Definitions section.

Typically the carbocyclic and heterocyclic groups are monocyclic.

Preferably the carbocyclic and heterocyclic groups are aromatic.

The group R⁹ is typically unsubstituted phenyl or benzyl, or phenyl or benzyl substituted by 1, 2 or 3 substituents selected from halogen; hydroxy; trifluoromethyl; cyano; carboxy; C₁₋₄alkoxycarbonyl; C₁₋₄ acyloxy; amino; mono- or di-C₁₋₄ alkylamino; C₁₋₄ alkyl optionally substituted by halogen, hydroxy or C₁₋₂ alkoxy; C₁₋₄ alkoxy optionally substituted by halogen, hydroxy or C₁₋₂ alkoxy; phenyl, five and six membered heteroaryl groups containing up to 3 heteroatoms selected from O, N and S; and saturated carbocyclic and heterocyclic groups containing up to 2 heteroatoms selected from O, S and N.

Examples of the moiety:

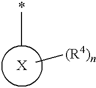

are set out in Table 6. The asterisk marks the point of attachment to the group E.

TABLE 6

| | |
|---|---|
| 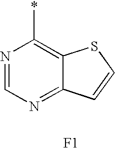 | 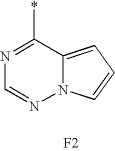 |
| 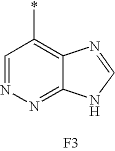 | 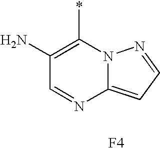 |
| 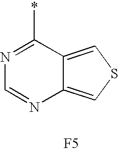 | 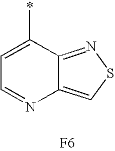 |
| 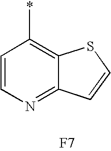 | 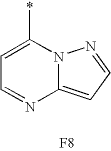 |
| 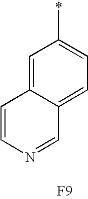 | 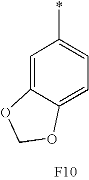 |
| 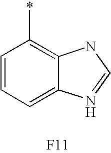 | 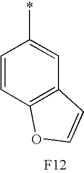 |
| 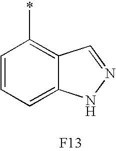 | 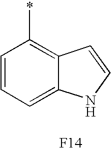 |
| 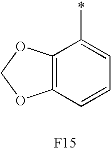 | 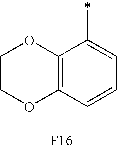 |
| 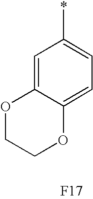 | 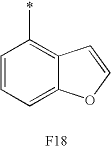 |
| 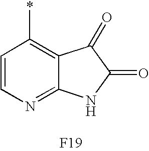 | 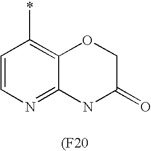 |

Preferred groups include F1, F19 and F20.

A particularly preferred group is F1.

### Particular and Preferred Sub-Groups of the Formula (I)

One sub-group of compounds of the formula (I) has the general formula (II):

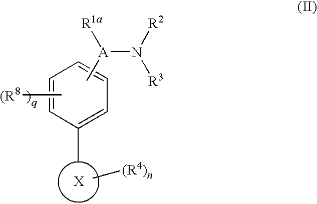

and salts, solvates, tautomers and N-oxides thereof,
wherein the group A is attached to the meta or para position of the benzene ring, q is 0-4;
R¹, R², R³, R⁴ and R⁵ are as defined herein in respect of formula (I) and sub-groups, examples and preferences thereof; and R⁸ is a substituent group as hereinbefore defined. In formula (II), q is preferably 0, 1 or 2, more preferably 0 or 1 and most preferably 0. Preferably the group A is attached to the para position of the benzene ring.

For example, one sub-group of the compounds of the formula (II) can be represented by the formula (IIa):

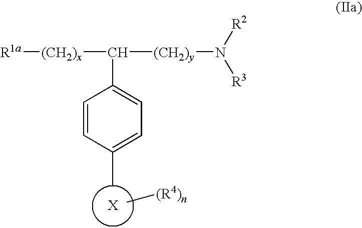

and salts, solvates, tautomers and N-oxides thereof,
wherein X, R^{1a}, R², R³, R⁴ and n are as defined herein, x is 0 or 1 and y is 0, 1 or 2 and. In one embodiment, both x and y are 1. In another embodiment, x is 0 and y is 1.

Another sub-group of compounds within formula (II) can be represented by the formula (IIb):

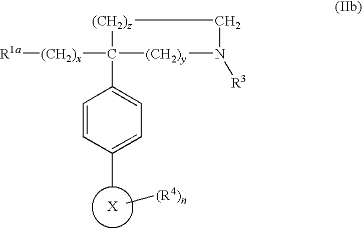

and salts, solvates, tautomers and N-oxides thereof,
wherein X, R^{1a}, R², R³, R⁴, x and y are as hereinbefore defined and z is 0, 1 or 2 provided that the sum of y and z does not exceed 4. In one particular embodiment, y is 2 and z is 1.

Another sub-group of compounds of the formula (I) has the general formula (IIc):

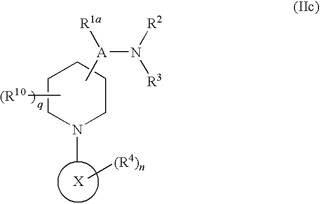

and salts, solvates, tautomers and N-oxides thereof,
wherein the group A is attached to the 3-position or 4-position of the piperidine ring, q is 0-4; and X, n, A, R^{1a}, R², R³ and R⁴ are as defined herein in respect of formula (I) and sub-groups, examples and preferences thereof; and R¹⁰ is a substituent group as hereinbefore defined. In formula (IIc), q is preferably 0, 1 or 2, more preferably 0 or 1 and most preferably 0 and/or n is preferably 0.

Another sub-group of compounds of the formula (I) has the general formula (III):

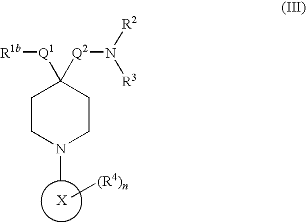

and salts, solvates, tautomers and N-oxides thereof,
wherein X, R^{1b}, R⁴, Q¹, Q² and NR²R³ are as defined herein in respect of formula (I) and sub-groups, examples and preferences thereof. Within Formula (III), particular compounds are those in which Q¹ is a bond or a C₁₋₂ alkylene group and Q² is a bond or a methylene group. Preferably R^{1b} is an aryl or heteroaryl group R^{1a}.

In formulae (III), preferably NR²R³ is NH₂ or NHMe.

Another group of preferred compounds of the invention can be represented by the formula (IV):

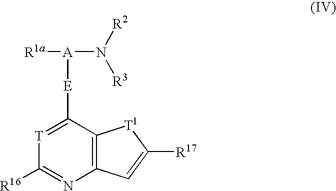

and salts, solvates, tautomers and N-oxides thereof,
wherein T¹ is S, O or NR¹⁸; R¹⁷ is hydrogen or a group R⁴; R¹⁸ is hydrogen or C₁₋₄ alkyl; and A, E, T, R^{1a} to R⁴ and R¹⁶ are as defined herein.

Within formula (IV), in one sub-group of compounds, T is N and T¹, is selected from S, O and NH. In a preferred group of compounds, T¹ is S.

In formulae (II), (IIa), (IIb), (IIc), (III) and (IV) and embodiments thereof, the groups R^{1a} and R^{1b} are each preferably an optionally substituted aryl or heteroaryl group, and typically a monocyclic aryl or heteroaryl group of 5 or 6 ring members. Particular aryl and heteroaryl groups are phenyl, pyridyl, furanyl and thienyl groups, each optionally substituted. Optionally substituted phenyl groups are particularly preferred.

Alternatively, the groups R^{1a} and R^{1b} can be, for example, an optionally substituted naphthyl group, for example an optionally substituted 1-naphthyl group. One particular example of such a group is unsubstituted 1-naphthyl.

The aryl or heteroaryl group (e.g. a phenyl, pyridyl, furanyl or thienyl group) can be unsubstituted or substituted by up to 5 substituents.

Particular sub-groups of compounds of the formulae (II), IIa), (IIb), (IIc) or (III) consist of compounds in which R^{1a} and R^{1b} are each is unsubstituted phenyl or, more preferably, phenyl bearing 1 to 3 (and more preferably 1 or 2) substituents selected from hydroxy; C₁₋₄ acyloxy; fluorine; chlorine; bromine; trifluoromethyl; cyano; C₁₋₄ hydrocarbyloxy and C₁₋₄ hydrocarbyl groups wherein the C₁₋₄ hydrocarbyloxy and C₁₋₄ hydrocarbyl groups are each optionally substituted by one or more C₁₋₂ alkoxy, halogen, hydroxy or optionally substituted phenyl or pyridyl groups; C₁₋₄ acylamino; benzoylamino; pyrrolidinocarbonyl; piperidinocarbonyl; morpholinocarbonyl; piperazinocarbonyl; five and six membered heteroaryl groups containing one or two heteroatoms selected from N, O and S, the heteroaryl groups being optionally substituted by one or more C₁₋₄ alkyl substituents; optionally substituted phenyl; optionally substituted pyridyl; and optionally substituted phenoxy; wherein the optional substituent for the phenyl, pyridyl and phenoxy groups are 1, 2 or 3 substituents selected from C₁₋₂ acyloxy, fluorine, chlorine, bromine, trifluoromethyl, cyano, and C₁₋₂ hydrocarbyloxy and C₁₋₂ hydrocarbyl groups wherein the C₁₋₂ hydrocarbyloxy and C₁₋₂ hydrocarbyl groups are each optionally substituted by methoxy or hydroxy.

Although up to 5 substituents may be present, more typically there are 0, 1, 2, 3 or 4 substituents, preferably 0, 1, 2 or 3, and more preferably 0, 1 or 2.

In one embodiment within each of formulae (II), (IIa), (IIb), (IIc), (III) and (IV), R^{1a} and R^{1b} are each selected from is unsubstituted phenyl or a phenyl group substituted by 1 or 2 substituents independently selected from hydroxy; C₁₋₄ acyloxy; fluorine; chlorine; bromine; trifluoromethyl; trifluoromethoxy; difluoromethoxy; benzyloxy; cyano; C₁₋₄ hydrocarbyloxy and C₁₋₄ hydrocarbyl each optionally substituted by C₁₋₂ alkoxy or hydroxy. More preferably, R^{1a} and R^{1b} are each selected from a substituted phenyl group bearing 1 or 2 substituents independently selected from fluorine; chlorine; trifluoromethyl; trifluoromethoxy; difluoromethoxy; cyano; methoxy, ethoxy, i-propoxy, methyl, ethyl, propyl, isopropyl, tert-butyl and benzyloxy.

In one particular sub-group of compounds within each of formulae (II), (IIa), (IIb), (IIc), (III) and (IV), the groups R^{1a} and R^{1b} are each a monosubstituted phenyl group having a chlorine substituent at the para position.

R¹⁷ is preferably hydrogen.

Another sub-group of compounds within Formula (III) has the general formula (V):

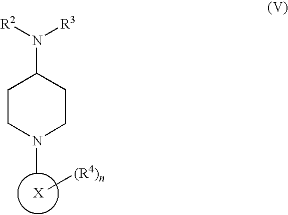

and salts, solvates, tautomers and N-oxides thereof,
wherein X, R², R³ and R⁴ are as defined herein in respect of formula (I) and sub-groups, examples and preferences thereof.

Another sub-group of compounds within formula (III) has the general formula (VI):

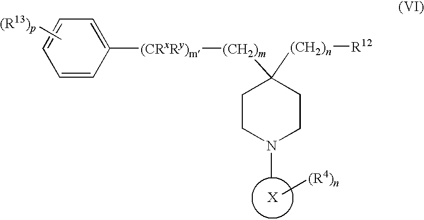

and salts, solvates, tautomers and N-oxides thereof,
wherein m is 0, 1 or 2; m′ is 0 or 1 provided that the sum of m and m′ is in the range 0 to 2; n is 0 or 1; p is 0, 1, 2 or 3; R^{x} and R^{y} are the same or different and each is selected from hydrogen, methyl and fluorine; R¹² is CN or NR²R³ and each R¹³ is independently selected from R¹⁰ wherein X, R², R³, R⁴ and R¹⁰ are as defined herein.

In formula (VI), m is preferably 0 or 1. When m′ is 0, more preferably m is 1. When m′ is 1, preferably m is 0.

In one group of compounds n is 0. In another group of compounds, n is 1.

Preferably p is 0, 1 or 2 and R¹³ is selected from hydroxy; C₁₋₄ acyloxy; fluorine; chlorine; bromine; trifluoromethyl; trifluoromethoxy; difluoromethoxy; benzyloxy; cyano; C₁₋₄ hydrocarbyloxy and C₁₋₄ hydrocarbyl each optionally substituted by C₁₋₂ alkoxy or hydroxy.

More preferably, R¹³ is selected from fluorine; chlorine; trifluoromethyl; trifluoromethoxy; difluoromethoxy; cyano; methoxy, ethoxy, i-propoxy, methyl, ethyl, propyl, isopropyl, tert-butyl and benzyloxy.

For example the phenyl group may have a substituent R¹³ at the para position selected from fluorine, chlorine, trifluoromethyl, trifluoromethoxy, difluoromethoxy, benzyloxy, methyl, tert-butyl and methoxy, and optionally a second substituent at the ortho- or meta-position selected from fluorine, chlorine or methyl. Within this sub-group, the phenyl group can be monosubstituted. Alternatively, the phenyl group can be disubstituted.

In another sub-group of compounds, p is 1 and the substituent R¹³ is a chlorine substituent at the para position.

In another sub-group of compounds, p is 2 and the phenyl group is a dichlorophenyl group, particular examples of which are 2,4-dichlorophenyl, 2,5-dichlorophenyl, 3,4-dichlorophenyl and 2,3-dichlorophenyl.

In one sub-group of compounds within formula (VI), R¹² is NR²R³ and more preferably R¹² is selected from NH₂, NHMe and NMe₂, with NH₂ being particularly preferred.

One particular sub-group of compounds within formula (VI) can be represented by the formula (VII):

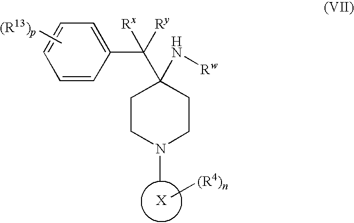

and salts, solvates, tautomers and N-oxides thereof,
wherein R^{x}, R^{y} and R^{w} are each independently hydrogen or methyl, and X, n, p, R⁴ and R¹³ are as defined herein.

In one embodiment, R^{w} is hydrogen. In another embodiment, R^{w} is methyl. Preferably, p is 0, 1 or 2. Preferably R^{x} and R^{y} are both hydrogen.

Alternatively, R^{x} and R^{y} may both be methyl, or may both be fluorine, or one of R^{x} and R^{y} may be hydrogen and the other may be methyl or fluorine.

Another sub-group of compounds within formula (III) can be represented by formula (VIII)

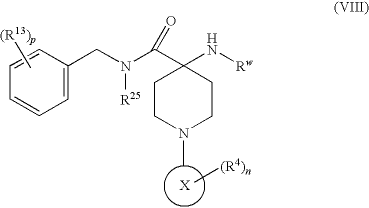

and salts, solvates, tautomers and N-oxides thereof,
wherein R²⁵ is hydrogen or methyl and X, R¹³, R⁴ and R^{w} are as defined herein.

Preferably, p is 0, 1 or 2.

In one group of compounds, R²⁵ is hydrogen. In another group of compounds, R²⁵ is methyl.

In one embodiment, R^{w} is hydrogen. In another embodiment, R^{w} is methyl.

Particular compounds within formulae (VII) and (VIII) are those wherein n is 0.

In each of formulae (VII) and (VIII), one group of preferred substituents R¹³ consists of chlorine, fluorine, methyl, ethyl, isopropyl, methoxy, difluoromethoxy, trifluoromethoxy, trifluoromethyl, tert-butyl, cyano and benzyloxy.

In formulae (VII) and (VIII), p is preferably 1 or 2.

In one embodiment, p is 1.

In another embodiment, p is 2.

When p is 1, the phenyl ring can be 2-substituted, or 3-substituted, or 4-substituted.

Particular examples of groups wherein p is 1 are the groups B2, B3, B5, B6, B8, B9, B10, B11, B12, B15, B18 and B19 in Table 2 above. More preferred groups are groups B2, B5, B10, B11, B15, B18 and B19 in Table 2.

When p is 2, the phenyl ring can be, for example, 2,3-disubstituted, 2,4-disubsubstituted, or 2,5-disubstituted.

Particular examples of groups wherein p is 2 are the groups B4, B7, B13, B14, B16, B17 and B20 in Table 2.

Another sub-group of compounds of the invention can be represented by the formula (IX):

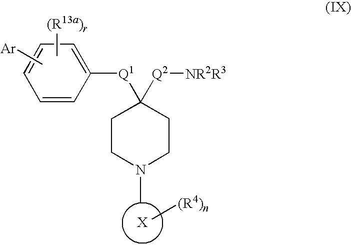

and salts, solvates, tautomers and N-oxides thereof,
wherein Ar is a 5- or 6-membered monocyclic aryl or heteroaryl group having up to 2 heteroatom ring members selected from O, N and S and being optionally substituted by one or two substituents selected from fluorine, chlorine, methyl and methoxy; R^{13a} is a substituent selected from fluorine, chlorine, methyl, trifluoromethyl, trifluoromethoxy and methoxy; r is 0, 1 or 2 (more typically 0 or 1); X, Q¹, Q², NR²R³ and R⁴ are as defined herein.

In formula (IX), particular 5- or 6-membered monocyclic aryl or heteroaryl groups Ar can be selected from phenyl, pyridyl, furyl and thienyl, each optionally substituted as defined herein. One particular monocyclic aryl group is optionally substituted phenyl, with unsubstituted phenyl being a particular example.

Within formula (IX), preferred compounds are those compounds wherein NR²R³ is selected from NH₂, NHMe and NMe₂ (with NH₂ being particularly preferred); and/or R⁴ is hydrogen or methyl (more preferably hydrogen); and/or Q¹ is selected from CH₂ and CH₂NHCO (wherein the carbonyl group is attached to the piperidine ring); and/or Q² is selected from CH₂ and a bond (and more preferably is a bond).

For the avoidance of doubt, it is to be understood that each general and specific preference, embodiment and example of the groups R^{1a} and R^{1b} may be combined with each general and specific preference, embodiment and example of the groups X and/or Q¹ and/or Q² and/or R² and/or R³ and/or R⁴ and/or R⁵ and/or R⁹ and that all such combinations are embraced by this application.

The various functional groups and substituents making up the compounds of the formula (I) are typically chosen such that the molecular weight of the compound of the formula (I) does not exceed 1000. More usually, the molecular weight of the compound will be less than 750, for example less than 700, or less than 650, or less than 600, or less than 550. More preferably, the molecular weight is less than 525 and, for example, is 500 or less.

Particular compounds of the invention are as illustrated in the examples below.

### Salts, Solvates, Tautomers, Isomers, N-Oxides, Esters, Prodrugs and Isotopes

In this section, as in all other sections of this application, unless the context indicates otherwise, references to formula (I) included references to formulae (II), (IIa), (IIb), (IIc), (III), (IV), (V), (VI), (VII), (VIII) and (IX) and all other sub-groups, preferences and examples thereof as defined herein.

Unless otherwise specified, a reference to a particular compound also includes ionic, salt, solvate, and protected forms thereof, for example, as discussed below.

Many compounds of the formula (I) can exist in the form of salts, for example acid addition salts or, in certain cases salts of organic and inorganic bases such as carboxylate, sulphonate and phosphate salts. All such salts are within the scope of this invention, and references to compounds of the formula (I) include the salt forms of the compounds. As in the preceding sections of this application, all references to formula (I) should be taken to refer also to formula (II) and sub-groups thereof unless the context indicates otherwise.

Salt forms may be selected and prepared according to methods described in Pharmaceutical Salts Properties, Selection, and Use, P. Heinrich Stahl (Editor), Camille G. Wermuth (Editor), ISBN: 3-90639-026-8, Hardcover, 388 pages, August 2002. For example, acid addition salts may be prepared by dissolving the free base in an organic solvent in which a given salt form is insoluble or poorly soluble and then adding the required acid in an appropriate solvent so that the salt precipitates out of solution.

Acid addition salts may be formed with a wide variety of acids, both inorganic and organic. Examples of acid addition salts include salts formed with an acid selected from the group consisting of acetic, 2,2-dichloroacetic, adipic, alginic, ascorbic (e.g. L-ascorbic), L-aspartic, benzenesulphonic, benzoic, 4-acetamidobenzoic, butanoic, (+) camphoric, camphor-sulphonic, (+)-(1S)-camphor-10-sulphonic, capric, caproic, caprylic, cinnamic, citric, cyclamic, dodecylsulphuric, ethane-1,2-disulphonic, ethanesulphonic, 2-hydroxyethanesulphonic, formic, fumaric, galactaric, gentisic, glucoheptonic, D-gluconic, glucuronic (e.g. D-glucuronic), glutamic (e.g. L-glutamic), α-oxoglutaric, glycolic, hippuric, hydrobromic, hydrochloric, hydriodic, isethionic, lactic (e.g. (+)-L-lactic and (±) DL-lactic), lactobionic, maleic, malic, (−)-L-malic, malonic, (±)-DL-mandelic, methanesulphonic, naphthalenesulphonic (e.g. naphthalene-2-sulphonic), naphthalene-1,5-disulphonic, 1-hydroxy-2-naphthoic, nicotinic, nitric, oleic, orotic, oxalic, palmitic, pamoic, phosphoric, propionic, L-pyroglutamic, salicylic, 4-amino-salicylic, sebacic, stearic, succinic, sulphuric, tannic, (+)-L-tartaric, thiocyanic, toluenesulphonic (e.g. p-toluenesulphonic), undecylenic and valeric acids, as well as acylated amino acids and cation exchange resins.

One particular group of acid addition salts includes salts formed with hydrochloric, hydriodic, phosphoric, nitric, sulphuric, citric, lactic, succinic, maleic, malic, isethionic, fumaric, benzenesulphonic, toluenesulphonic, methanesulphonic, ethanesulphonic, naphthalenesulphonic, valeric, acetic, propanoic, butanoic, malonic, glucuronic and lactobionic acids.

Another group of acid addition salts includes salts formed from acetic, adipic, ascorbic, aspartic, citric, DL-Lactic, fumaric, gluconic, glucuronic, hippuric, hydrochloric, glutamic, DL-malic, methanesulphonic, sebacic, stearic, succinic and tartaric acids.

The compounds of the invention may exist as mono- or di-salts depending upon the pKa of the acid from which the salt is formed.

If the compound is anionic, or has a functional group which may be anionic (e.g., —COOH may be —COO⁻), then a salt may be formed with a suitable cation. Examples of suitable inorganic cations include, but are not limited to, alkali metal ions such as Na⁺ and K⁺, alkaline earth cations such as Ca²⁺ and Mg²⁺, and other cations such as Al³⁺. Examples of suitable organic cations include, but are not limited to, ammonium ion (i.e., NH₄⁺) and substituted ammonium ions (e.g., NH₃R⁺, NH₂R₂⁺, NHR₃⁺, NR₄⁺). Examples of some suitable substituted ammonium ions are those derived from: ethylamine, diethylamine, dicyclohexylamine, triethylamine, butylamine, ethylenediamine, ethanolamine, diethanolamine, piperazine, benzylamine, phenylbenzylamine, choline, meglumine, and tromethamine, as well as amino acids, such as lysine and arginine. An example of a common quaternary ammonium ion is N(CH₃)₄⁺.

Where the compounds of the formula (I) contain an amine function, these may form quaternary ammonium salts, for example by reaction with an alkylating agent according to methods well known to the skilled person. Such quaternary ammonium compounds are within the scope of formula (I).

Compounds of the formula (I) containing an amine function may also form N-oxides. A reference herein to a compound of the formula (I) that contains an amine function also includes the N-oxide.

Where a compound contains several amine functions, one or more than one nitrogen atom may be oxidised to form an N-oxide. Particular examples of N-oxides are the N-oxides of a tertiary amine or a nitrogen atom of a nitrogen-containing heterocycle.

N-Oxides can be formed by treatment of the corresponding amine with an oxidizing agent such as hydrogen peroxide or a per-acid (e.g. a peroxycarboxylic acid), see for example Advanced Organic Chemistry, by Jerry March, 4^{th} Edition, Wiley Interscience, pages. More particularly, N-oxides can be made by the procedure of L. W. Deady (Syn. Comm. 1977, 7, 509-514) in which the amine compound is reacted with m-chloroperoxybenzoic acid (MCPBA), for example, in an inert solvent such as dichloromethane.

Compounds of the formula (I) may exist in a number of different geometric isomeric, and tautomeric forms and references to compounds of the formula (I) include all such forms. For the avoidance of doubt, where a compound can exist in one of several geometric isomeric or tautomeric forms and only one is specifically described or shown, all others are nevertheless embraced by formula (I).

Where compounds of the formula (I) contain one or more chiral centres, and can exist in the form of two or more optical isomers, references to compounds of the formula (I) include all optical isomeric forms thereof (e.g. enantiomers and diastereoisomers), either as individual optical isomers, or mixtures or two or more optical isomers, unless the context requires otherwise.

For example, the group A can include one or more chiral centres. Thus, when E and R¹ are both attached to the same carbon atom on the linker group A, the said carbon atom is typically chiral and hence the compound of the formula (I) will exist as a pair of enantiomers (or more than one pair of enantiomers where more than one chiral centre is present in the compound).

The optical isomers may be characterised and identified by their optical activity (i.e. as + and − isomers) or they may be characterised in terms of their absolute stereochemistry using the “R and S” nomenclature developed by Cahn, Ingold and Prelog, see Advanced Organic Chemistry by Jerry March, 4^{th} Edition, John Wiley & Sons, New York, 1992, pages 109-114, and see also Cahn, Ingold & Prelog, Angew. Chem. Int. Ed. Engl., 1966, 5, 385-415.

Optical isomers can be separated by a number of techniques including chiral chromatography (chromatography on a chiral support) and such techniques are well known to the person skilled in the art.

As an alternative to chiral chromatography, optical isomers can be separated by forming diastereoisomeric salts with chiral acids such as (+)-tartaric acid, (−)-pyroglutamic acid, (−)-di-toluloyl-L-tartaric acid, (+)-mandelic acid, (−)-malic acid, and (−)-camphorsulphonic, separating the diastereoisomers by preferential crystallisation, and then dissociating the salts to give the individual enantiomer of the free base.

Where compounds of the formula (I) exist as two or more optical isomeric forms, one enantiomer in a pair of enantiomers may exhibit advantages over the other enantiomer, for example, in terms of biological activity. Thus, in certain circumstances, it may be desirable to use as a therapeutic agent only one of a pair of enantiomers, or only one of a plurality of diastereoisomers. Accordingly, the invention provides compositions containing a compound of the formula (I) having one or more chiral centres, wherein at least 55% (e.g. at least 60%, 65%, 70%, 75%, 80%, 85%, 90% or 95%) of the compound of the formula (I) is present as a single optical isomer (e.g. enantiomer or diastereoisomer). In one general embodiment, 99% or more (e.g. substantially all) of the total amount of the compound of the formula (I) may be present as a single optical isomer (e.g. enantiomer or diastereoisomer).

Esters such as carboxylic acid esters and acyloxy esters of the compounds of formula (I) bearing a carboxylic acid group or a hydroxyl group are also embraced by Formula (I). In one embodiment of the invention, formula (I) includes within its scope esters of compounds of the formula (I) bearing a carboxylic acid group or a hydroxyl group. In another embodiment of the invention, formula (I) does not include within its scope esters of compounds of the formula (I) bearing a carboxylic acid group or a hydroxyl group. Examples of esters are compounds containing the group —C(═O)OR, wherein R is an ester substituent, for example, a C₁₋₇ alkyl group, a C₃₋₂₀ heterocyclyl group, or a C₅₋₂₀ aryl group, preferably a C₁₋₇ alkyl group. Particular examples of ester groups include, but are not limited to, —C(═O)OCH₃, —C(═O)OCH₂CH₃, —C(═O)OC(CH₃)₃, and —C(═O)OPh. Examples of acyloxy (reverse ester) groups are represented by —OC(═O)R, wherein R is an acyloxy substituent, for example, a C₁₋₇ alkyl group, a C₃₋₂₀ heterocyclyl group, or a C₅₋₂₀ aryl group, preferably a C₁₋₇ alkyl group. Particular examples of acyloxy groups include, but are not limited to, —OC(═O)CH₃ (acetoxy), —OC(═O)CH₂CH₃, —OC(═O)C(CH₃)₃, —OC(═O)Ph, and —OC(═O)CH₂Ph.

Also encompassed by formula (I) are any polymorphic forms of the compounds, solvates (e.g. hydrates), complexes (e.g. inclusion complexes or clathrates with compounds such as cyclodextrins, or complexes with metals) of the compounds, and pro-drugs of the compounds. By “prodrugs” is meant for example any compound that is converted in vivo into a biologically active compound of the formula (I).

For example, some prodrugs are esters of the active compound (e.g., a physiologically acceptable metabolically labile ester). During metabolism, the ester group (—C(═O)OR) is cleaved to yield the active drug. Such esters may be formed by esterification, for example, of any of the carboxylic acid groups (—C(═O)OH) in the parent compound, with, where appropriate, prior protection of any other reactive groups present in the parent compound, followed by deprotection if required.

Examples of such metabolically labile esters include those of the formula —C(═O)OR wherein R is:

C₁₋₇alkyl (e.g., -Me, -Et, -nPr, -iPr, -nBu, -sBu, -iBu, -tBu); C₁₋₇ aminoalkyl (e.g., aminoethyl; 2-(N,N-diethylamino)ethyl; 2-(4-morpholino)ethyl); and acyloxy-C₁₋₇alkyl (e.g., acyloxymethyl; acyloxyethyl; pivaloyloxymethyl; acetoxymethyl; 1-acetoxyethyl; 1-(1-methoxy-1-methyl)ethyl-carbonyloxyethyl; 1-(benzoyloxy)ethyl; isopropoxy-carbonyloxymethyl; 1-isopropoxy-carbonyloxyethyl; cyclohexyl-carbonyloxymethyl; 1-cyclohexyl-carbonyloxyethyl; cyclohexyloxy-carbonyloxymethyl; 1-cyclohexyloxy-carbonyloxyethyl; (4-tetrahydropyranyloxy)carbonyloxymethyl; 1-(4-tetrahydropyranyloxy)-carbonyloxyethyl; (4-tetrahydropyranyl)carbonyloxymethyl; and 1-(4-tetrahydropyranyl)-carbonyloxyethyl).

Also, some prodrugs are activated enzymatically to yield the active compound, or a compound which, upon further chemical reaction, yields the active compound (for example, as in antigen-directed enzyme pro-drug therapy (ADEPT), gene-directed enzyme pro-drug therapy (GDEPT) and ligand-directed enzyme pro-drug therapy (LIDEPT). For example, the prodrug may be a sugar derivative or other glycoside conjugate, or may be an amino acid ester derivative.

### Methods for the Preparation of Compounds of the Formula (I)

In this section, as in all other sections of this application, unless the context indicates otherwise, references to formula (I) included references to formulae (II), (IIa), (IIb), (IIc), (III), (IV), (V), (VI), (VII), (VIII) and (IX) and all other sub-groups, preferences and examples thereof as defined herein.

Compounds of the formula (I) wherein TG is

(1) a group:

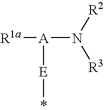

can be prepared by reaction of a compound of the formula (X) with a compound of the formula (XI) or an N-protected derivative thereof:

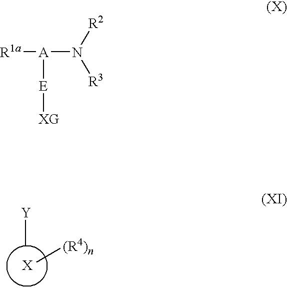

wherein X, XG, A, E, n and R^{1a} to R⁴ are as hereinbefore defined, one of the groups XG and Y is chlorine, bromine or iodine or a trifluoromethanesulphonate (triflate) group, and the other one of the groups XG and Y is a boronate residue, for example a boronate ester or boronic acid residue.

The reaction can be carried out under typical Suzuki Coupling conditions in the presence of a palladium catalyst such as bis(tri-t-butylphosphine)palladium and a base (e.g. a carbonate such as potassium carbonate). The reaction may be carried out in an aqueous solvent system, for example aqueous ethanol, and the reaction mixture is typically subjected to heating, for example to a temperature in excess of 100° C.

An illustrative synthetic route involving a Suzuki coupling step is shown in Scheme 1. The starting material for the synthetic route shown in scheme 1 is the halo-substituted aryl- or heteroarylmethyl nitrile (XII) in which X is a chlorine, bromine or iodine atom or a triflate group. The nitrile (XII) is condensed with the aldehyde R¹CHO in the presence of an alkali such as sodium or potassium hydroxide in an aqueous solvent system such as aqueous ethanol. The reaction can be carried out at room temperature.

The resulting substituted acrylonitrile derivative (XIII) is then treated with a reducing agent that will selectively reduce the alkene double bond without reducing the nitrile group. A borohydride such as sodium borohydride may be used for this purpose to give the substituted acetonitrile derivative (XIV). The reduction reaction is typically carried out in a solvent such as ethanol and usually with heating, for example to a temperature up to about 65° C.

The reduced nitrile (XIV) is then coupled with the boronate ester (XV) under the Suzuki coupling conditions described above to give a compound of the formula (I) in which A-NR²R³ is a substituted acetonitrile group.

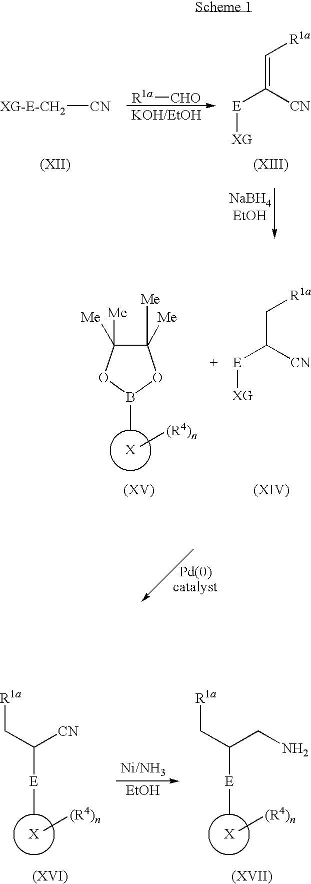

The substituted acetonitrile compound (XVI) may then be reduced to the corresponding amine (XVII) by treatment with a suitable reducing agent such as Raney nickel and ammonia in ethanol.

The synthetic route shown in Scheme 1 gives rise to amino compounds of the formula (I) in which the aryl or heteroaryl group E is attached to the β-position of the group A relative to the amino group. In order to give amino compounds of the formula (I) in which R¹ is attached to the β-position relative to the amino group, the functional groups on the two starting materials in the condensation step can be reversed so that a compound of the formula XG-E-CHO wherein XG is bromine, chlorine, iodine or a triflate group is condensed with a compound of the formula R^{1a}—CH₂—CN to give a substituted acrylonitrile derivative which is then reduced to the corresponding acetonitrile derivative before coupling with the boronate (XV) and reducing the cyano group to an amino group.

Compounds of the formula (I) in which R^{1a} is attached to the α-position relative to the amino group can be prepared by the sequence of reactions shown in Scheme 2.

In Scheme 2, the starting material is a halo-substituted aryl- or heteroarylmethyl Grignard reagent (XVIII, X=bromine or chlorine) which is reacted with the nitrile R^{1a}—CN in a dry ether such as diethyl ether to give an intermediate imine (not shown) which is reduced to give the amine (XIX) using a reducing agent such as lithium aluminium hydride. The amine (XIX) can be reacted with the boronate ester (XV) under the Suzuki coupling conditions described above to yield the amine (XX).

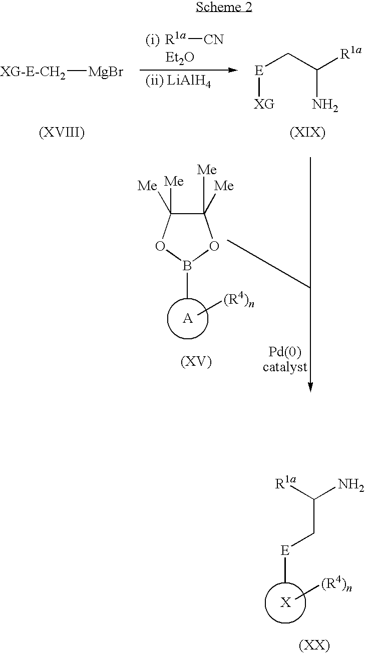

Compounds of the formula (I) can also be prepared from the substituted nitrile compound (XXI).

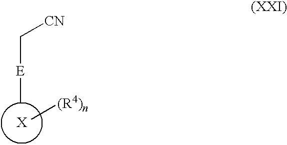

The nitrile (XXI) can be condensed with an aldehyde of the formula R^{1a}—(CH₂)ᵣ—CHO, wherein r is 0 or 1, and the resulting substituted acrylonitrile subsequently reduced to the corresponding substituted nitrile under conditions analogous to those set out in Scheme 1 above. The protecting group PG can then be removed by an appropriate method. The nitrile compound may subsequently be reduced to the corresponding amine by the use of a suitable reducing agent as described above.

The nitrile compound (XXI) may also be reacted with a Grignard reagent of the formula R^{1a}—(CH₂)ᵣ—MgBr under standard Grignard reaction conditions followed by deprotection to give an amino compound of the invention which has the structure shown in formula (XXII).

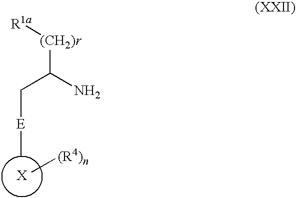

In the preparative procedures outlined above, the group E and the cyclic group X are coupled together by the reaction of a halo-aryl or heteroaryl compound with a boronate ester or boronic acid in the presence of a palladium catalyst and base. Many boronates suitable for use in preparing compounds of the invention are commercially available, for example from Boron Molecular Limited of Noble Park, Australia, or from Combi-Blocks Inc, of San Diego, USA. Where the boronates are not commercially available, they can be prepared by methods known in the art, for example as described in the review article by N. Miyaura and A. Suzuki, Chem. Rev. 1995, 95, 2457. Thus, boronates can be prepared by reacting the corresponding bromo-compound with an alkyl lithium such as butyl lithium and then reacting with a borate ester. The resulting boronate ester derivative can, if desired, be hydrolysed to give the corresponding boronic acid.

Compounds of the formula (I) in which the group A contains a nitrogen atom attached to the group E can be prepared by well known synthetic procedures from compounds of the formula (XXIII) or a protected form thereof. Compounds of the formula (XXIII) can be obtained by a Suzuki coupling reaction of a compound of the formula (XV) (see Scheme 1) with a compound of the formula Br-E-NH₂ such as 4-bromoaniline.

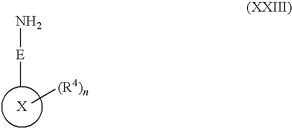

Compounds of the formula (I) in which R^{1a} and E are connected to the same carbon atom can be prepared as shown in Scheme 3.

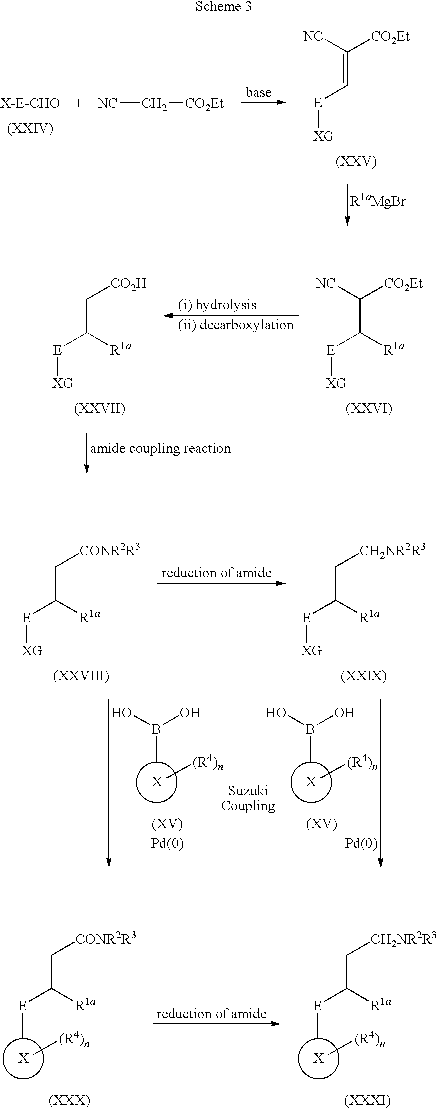

In Scheme 3, an aldehyde compound (XXIV) where XG is bromine, chlorine, iodine or a triflate group is condensed with ethyl cyanoacetate in the presence of a base to give a cyanoacrylate ester intermediate (XXV). The condensation is typically carried out in the presence of a base, preferably a non-hydroxide such as piperidine, by heating under Dean Stark conditions.

The cyanoacrylate intermediate (XXV) is then reacted with a Grignard reagent R^{1a}MgBr suitable for introducing the group R^{1a} by Michael addition to the carbon-carbon double bond of the acrylate moiety. The Grignard reaction may be carried out in a polar non-protic solvent such as tetrahydrofuran at a low temperature, for example at around 0° C. The product of the Grignard reaction is the cyano propionic acid ester (XXVI) and this is subjected to hydrolysis and decarboxylation to give the propionic acid derivative (XXVII). The hydrolysis and decarboxylation steps can be effected by heating in an acidic medium, for example a mixture of sulphuric acid and acetic acid.

The propionic acid derivative (XXVII) is converted to the amide (XXVIII) by reaction with an amine HNR²R³ under conditions suitable for forming an amide bond. The coupling reaction between the propionic acid derivative (XXVII) and the amine HNR²R³ is preferably carried out in the presence of a reagent of the type commonly used in the formation of peptide linkages. Examples of such reagents include 1,3-dicyclohexylcarbodiimide (DCC) (Sheehan et al, J. Amer. Chem. Soc. 1955, 77, 1067), 1-ethyl-3-(3′-dimethylaminopropyl)-carbodiimide (referred to herein either as EDC or EDAC) (Sheehan et al, J. Org. Chem., 1961, 26, 2525), uronium-based coupling agents such as O-(7-azabenzotriazol-1-yl)-N,N,N′,N′-tetramethyluronium hexafluorophosphate (HATU) and phosphonium-based coupling agents such as 1-benzo-triazolyloxytris-(pyrrolidino)phosphonium hexafluorophosphate (PyBOP) (Castro et al, Tetrahedron Letters, 1990, 31, 205). Carbodiimide-based coupling agents are advantageously used in combination with 1-hydroxy-7-azabenzotriazole (HOAt) (L. A. Carpino, J. Amer. Chem. Soc., 1993, 115, 4397) or 1-hydroxybenzotriazole (HOBt) (Konig et al, Chem. Ber., 103, 708, 2024-2034). Preferred coupling reagents include EDC (EDAC) and DCC in combination with HOAt or HOBt.

The coupling reaction is typically carried out in a non-aqueous, non-protic solvent such as acetonitrile, dioxan, dimethylsulphoxide, dichloromethane, dimethylformamide or N-methylpyrrolidine, or in an aqueous solvent optionally together with one or more miscible co-solvents. The reaction can be carried out at room temperature or, where the reactants are less reactive (for example in the case of electron-poor anilines bearing electron withdrawing groups such as sulphonamide groups) at an appropriately elevated temperature. The reaction may be carried out in the presence of a non-interfering base, for example a tertiary amine such as triethylamine or N,N-diisopropylethylamine.

Where the amine HNR²R³ is ammonia, the amide coupling reaction can be carried out using 1,1′-carbonyldiimidazole (CDI) to activate the carboxylic acid before addition of the ammonia.

As an alternative, a reactive derivative of the carboxylic acid, e.g. an anhydride or acid chloride, may be used. Reaction with a reactive derivative such an anhydride is typically accomplished by stirring the amine and anhydride at room temperature in the presence of a base such as pyridine.

The amide (XXVIII) can be converted to a compound of the formula (XXX) (which corresponds to a compound of the formula (I) wherein A has an oxo substituent next to the NR²R³ group) by reaction with a boronate (XV) under Suzuki coupling conditions as described above. The amide (XXX) can subsequently be reduced using a hydride reducing agent such as lithium aluminium hydride in the presence of aluminium chloride to give an amine of the formula (XXXI) (which corresponds to a compound of the formula (I) wherein A is CH—CH₂—CH₂—). The reduction reaction is typically carried out in an ether solvent, for example diethyl ether, with heating to the reflux temperature of the solvent.

Rather than reacting the amide (XXVIII) with the boronate (XV), the amide may instead be reduced with lithium aluminium hydride/aluminium chloride, for example in an ether solvent at ambient temperature, to give the amine (XXIX) which is then reacted with the boronate (XV) under the Suzuki coupling conditions described above to give the amine (XXX).

In order to obtain the homologue of the amine (XXIX) containing one fewer methylene group, the carboxylic acid (XXVII) can be converted to the azide by standard methods and subjected to a Curtius rearrangement in the presence of an alcohol such as benzyl alcohol to give a carbamate (see Advanced Organic Chemistry, 4^{th} edition, by Jerry March, John Wiley & sons, 1992, pages 1091-1092). The benzylcarbamate can function as a protecting group for the amine during the subsequent Suzuki coupling step, and the benzyloxycarbonyl moiety in the carbamate group can then be removed by standard methods after the coupling step. Alternatively, the benzylcarbamate group can be treated with a hydride reducing agent such as lithium aluminium hydride to give a compound in which NR²R³ is a methylamino group instead of an amino group.

Intermediate compounds of the formula (X) where the moiety X is a chlorine, bromine or iodine atom and A is a group CH—CH₂— can be prepared by the reductive amination of an aldehyde compound of the formula (XXXII):

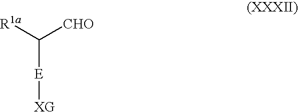

with an amine of the formula HNR²R³ under standard reductive amination conditions, for example in the presence of sodium cyanoborohydride in an alcohol solvent such as methanol or ethanol.

The aldehyde compound (XXXII) can be obtained by oxidation of the corresponding alcohol (XXXIII) using, for example, the Dess-Martin periodinane (see Dess, D. B.; Martin, J. C. J. Org. Soc., 1983, 48, 4155 and Organic Syntheses, Vol. 77, 141).

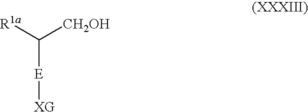

Compounds of the formula (I) where A, N and R² together form a cyclic group can be formed by the Suzuki coupling of a boronate compound of the formula (XV) with a cyclic intermediate of the formula (XXXIV) or an N-protected derivative thereof.

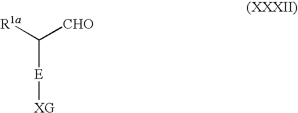

Cyclic intermediates of the formula (XXXIV), where R¹ is an aryl group such as an optionally substituted phenyl group, can be formed by Friedel Crafts alkylation of an aryl compound R¹—H with a compound of the formula (XXXV):

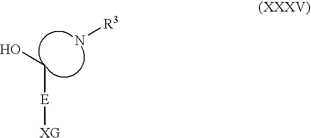

The alkylation is typically carried out in the presence of a Lewis acid such as aluminium chloride at a reduced temperature, for example less than 5° C.

The Friedel Crafts reaction has been found to be of general applicability to the preparation of a range of intermediates of the formula (X). Accordingly, in a general method of making compounds of the formula (X), a compound of the formula (LXX):

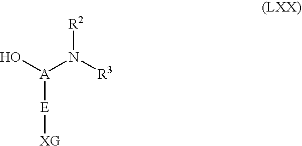

is reacted with a compound of the formula R^{1a}—H under Friedel Crafts alkylation conditions, for example in the presence of an aluminium, halide (e.g. AlCl₃).

In a further method for the preparation of a compound of the formula (I) wherein the moiety NR²R³ is attached to a CH₂ group of the moiety A, an aldehyde of the formula (XXXVI) can be coupled with an amine of the formula HNR²R³ under reductive amination conditions as described above. In the formulae (XXXVI) and (XXXVII), A′ is the residue of the group A—i.e. the moieties A′ and CH₂ together form the group A. The aldehyde (XXXVII) can be formed by oxidation of the corresponding alcohol using, for example, Dess-Martin periodinane.

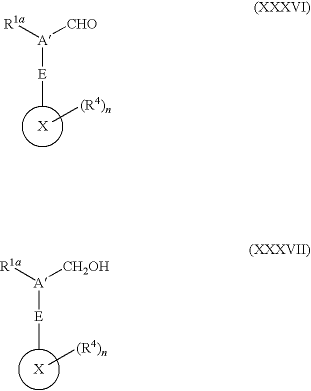

A Friedel Crafts alkylation procedure of the type described above for the synthesis of intermediates of the formula (XXXIV) can also be used to prepare intermediates of the formula (X) wherein XG is bromine. An example of such a procedure is shown in Scheme 4.

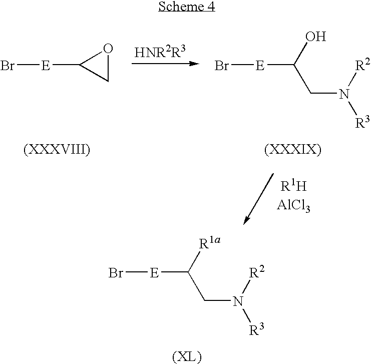

The starting material for the synthetic route shown in Scheme 4 is the epoxide (XXXVIII) which can either be obtained commercially or can be made by methods well known to the skilled person, for example by reaction of the aldehyde Br-E-CHO with trimethylsulphonium iodide. The epoxide (XXXVIII) is reacted with an amine HNR²R³ under conditions suitable for a ring-opening reaction with the epoxide to give a compound of the formula (XXXIX). The ring opening reaction can be carried out in a polar solvent such as ethanol at room temperature or optionally with mild heating, and typically with a large excess of the amine.

The amine (XXXIX) is then reacted with an aryl compound R^{1a}H, typically a phenyl compound, capable of taking part in a Friedel Crafts alkylation (see for example Advanced Organic Chemistry, by Jerry March, pages 534-542). Thus, the amine of formula (XXXIX) is typically reacted with the aryl compound R^{1a}H in the presence of an aluminium chloride catalyst at or around room temperature. Where the aryl compound R^{1a}H is a liquid, e.g. as in the case of a methoxybenzene (e.g. anisole) or a halobenzene such as chlorobenzene, the aryl compound may serve as the solvent. Otherwise, a less reactive solvent such as nitrobenzene may be used. The Friedel Crafts alkylation of the compound R¹H with the amine (XXXIX) gives a compound of the formula (XL) which corresponds to a compound of the formula (X) wherein XG is bromine and A is CHCH₂.

The hydroxy intermediate (XXXIX) in Scheme 4 can also be used to prepare compounds of the formula (X) in which the carbon atom of the hydrocarbon linker group A adjacent the group R^{1a} is replaced by an oxygen atom. Thus the compound of formula (XXXIX), or an N-protected derivative thereof (where R² or R³ are hydrogen) can be reacted with a phenolic compound of the formula R^{1a}—OH under Mitsunobu alkylation conditions, e.g. in the presence of diethyl azodicarboxylate and triphenylphosphine. The reaction is typically carried out in a polar non-protic solvent such as tetrahydrofuran at a moderate temperature such as ambient temperature.

A further use of the hydroxy-intermediate (XXXIX) is for the preparation of the corresponding fluoro-compound. Thus, the hydroxy group can be replaced by fluorine by reaction with pyridine:hydrogen fluoride complex (Olah's reagent). The fluorinated intermediate can then be subjected to a Suzuki coupling reaction to give a compound of the formula (I) with a fluorinated hydrocarbon group A. A fluorinated compound of the formula (I) could alternatively be prepared by first coupling the hydroxy intermediate (XXXIX), or a protected form thereof, with a heteroaryl boronic acid or boronate under Suzuki conditions and then replacing the hydroxy group in the resulting compound of formula (I) with fluorine using pyridine: hydrogen fluoride complex.

Compounds of the formula (I) in which the moiety:

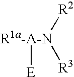

is a group:

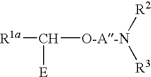

where A″ is the hydrocarbon residue of the group A, can be prepared by the sequence of reactions shown in Scheme 5.

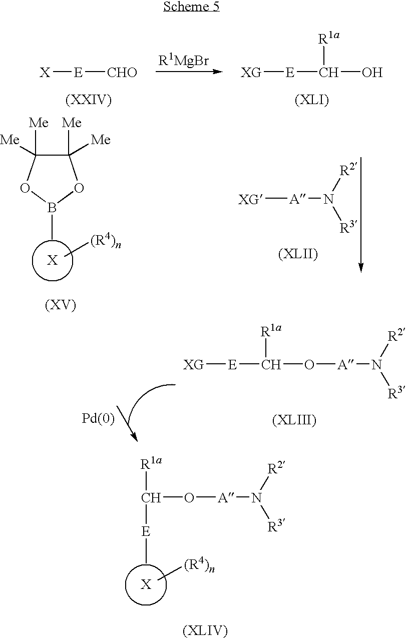

As shown in Scheme 5, the aldehyde (XXIV) is reacted with a Grignard reagent R^{1a}MgBr under standard Grignard conditions to give the secondary alcohol (XLI). The secondary alcohol can then be reacted with a compound of the formula (XLII) in which R^{2′} and R^{3′} represent the groups R² and R³ or an amine-protecting group, A″ is the residue of the group A, and XG′ represents a hydroxy group or a leaving group.

The amine protecting group can be, for example, a phthalolyl group in which case NR^{2′}R^{3′} is a phthalimido group.

When XG′ is a hydroxy group, the reaction between compound (XLI) and (XLII) can take the form of an toluene sulphonic acid catalysed condensation reaction. Alternatively, when XG′ is a leaving group such as halogen, the alcohol (XLI) can first be treated with a strong base such as sodium hydride to form the alcoholate which then reacts with the compound (XLII).

The resulting compound of the formula (XLIII) is then subjected to a Suzuki coupling reaction with the boronate reagent (XV) under typical Suzuki coupling conditions of the type described above to give a compound of the formula (XLIV). The protecting group can then be removed from the protected amine group NR^{2′}R^{3′}to give a compound of the formula (I).

Compounds of the formula (I) in which the moiety:

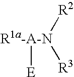

is a group:

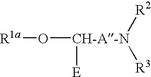

where A″ is the hydrocarbon residue of the group A, can be prepared by the sequence of reactions shown in Scheme 6.

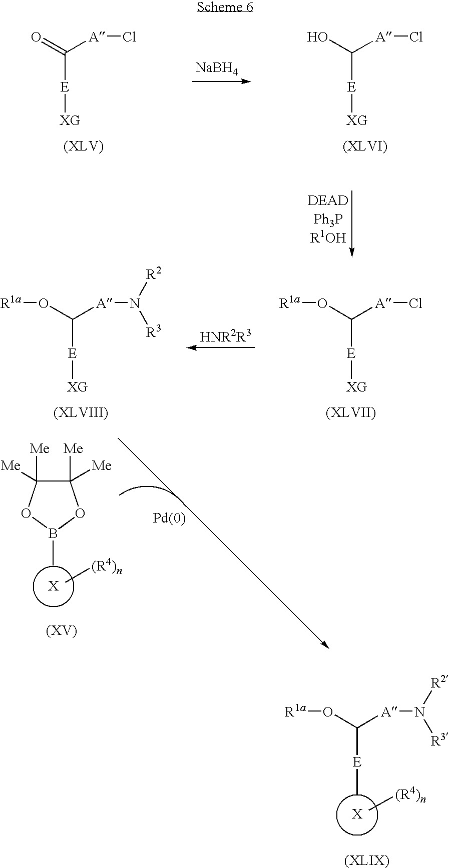

The starting material in Scheme 6 is the chloroacyl compound (XLV) which can be prepared by literature methods (e.g. the method described in J. Med. Chem., 2004, 47, 3924-3926) or methods analogous thereto. Compound (XLV) is converted into the secondary alcohol (XLVI) by reduction with a hydride reducing agent such as sodium borohydride in a polar solvent such as water/tetrahydrofuran.

The secondary alcohol (XLVI) can then be reacted with a phenolic compound of the formula R^{1a}—OH under Mitsunobu alkylation conditions, e.g. in the presence of diethyl azodicarboxylate and triphenylphosphine, as described above, to give the aryl ether compound (XLVII).

The chorine atom in the aryl ether compound (XLVII) is then displaced by reaction with an amine HNR²R³ to give a compound of the formula (XLVIII). The nucleophilic displacement reaction may be carried out by heating the amine with the aryl ether in a polar solvent such as an alcohol at an elevated temperature, for example approximately 100° C. The heating may advantageously be achieved using a microwave heater. The resulting amine (XLVIII) can then be subjected to a Suzuki coupling procedure with a boronate of the formula (XV) as described above to give the compound (XLIX).

In a variation on the reaction sequence shown in Scheme 6, the secondary alcohol (XLVI) can be subjected to a nucleophilic displacement reaction with an amine HNR²R³ before introducing the group R¹ by means of the Mitsunobu ether-forming reaction.

Another route to compounds of the formula (I) in which E and R^{1a} are attached to the same carbon atom in the group A is illustrated in Scheme 7.

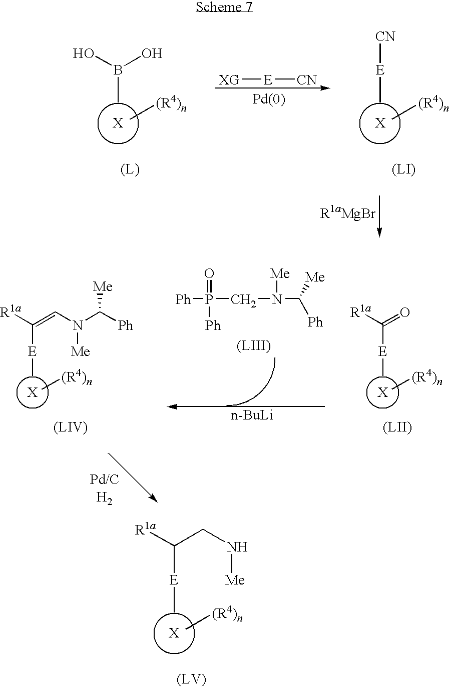

In Scheme 7, boronic acid compound (L) is reacted under Suzuki coupling conditions with the cyano compound XG-E-CN in which XG is typically a halogen such as bromine or chlorine. The boronic acid (L) can be prepared using the method described in EP 1382603 or methods analogous thereto.

The resulting nitrile (LI) may then be reacted with a Grignard reagent R^{1a}—MgBr to introduce the group R^{1a} and form the ketone (LII). The ketone (LII) is converted to the enamine (LIV) by reaction with the diphenylphosphinoylmethylamine (LIII) in the presence of a strong base such as an alkyl lithium, particularly butyl lithium.

The enamine (LIV) is then subjected to hydrogenation over a palladium on charcoal catalyst to reduce the double bond of the enamine and remove the 1-phenethyl group, thereby yielding a compound of the formula (LV).

Alternatively, the enamine (LIV) can be reduced with a hydride reducing agent under the conditions described in Tetrahedron: Asymmetry 14 (2003) 1309-1316 and subjected to a chiral separation. Removal of the protecting 2-phenethyl group then gives an optically active form of the compound of formula (LV).

Intermediates of the formula (X) wherein A and R² link to form a ring containing an oxygen atom can be prepared by the general method illustrated in Scheme 8.

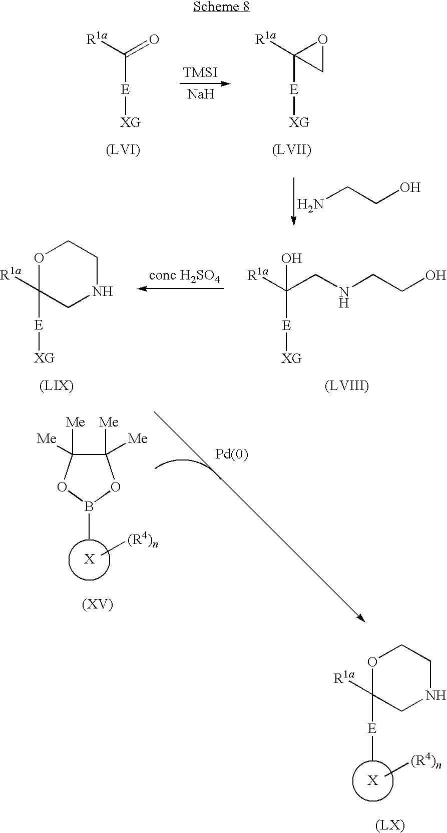

In Scheme 8, a ketone (LVI) is reacted with trimethylsulphonium iodide to form the epoxide (LVII). The reaction is typically carried out in the presence of a hydride base such as sodium hydride in a polar solvent such as dimethylsulphoxide.

The epoxide (LVII) is subjected to a ring opening reaction with ethanolamine in the presence of a non-interfering base such as triethylamine in a polar solvent such as an alcohol (e.g. isopropanol), usually with mild heating (e.g. up to approximately 50° C. The resulting secondary alcohol is then cyclised to form the morpholine ring by treatment with concentrated sulphuric acid in a solvent such as ethanolic dichloromethane.

The morpholine intermediate (LIX) can then reacted with the boronate (XV) under Suzuki coupling conditions to give the compound of formula (LX), which corresponds to a compound of the formula (I) in which A-NR²R³ forms a morpholine group.

Instead of reacting the epoxide (LVII) with ethanolamine, it may instead be reacted with mono- or dialkylamines thereby providing a route to compounds containing the moiety:

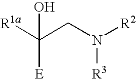

Compounds wherein R² and R³ are both hydrogen can be prepared by reacting the epoxide (LVII) with potassium phthalimide in a polar solvent such as DMSO. During the Suzuki coupling step, the phthalimide group may undergo partial hydrolysis to give the corresponding phthalamic acid which can be cleaved using hydrazine to give the amino group NH₂. Alternatively, the phthalamic acid can be recyclised to the phthalimide using a standard amide-forming reagent and the phthaloyl group then removed using hydrazine to give the amine.

A further synthetic route to compounds of the formula (I) wherein A and NR²R³ combine to form a cyclic group is illustrated in Scheme 9.

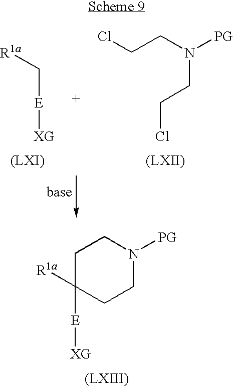

In Scheme 9, the starting material (LXI) is typically a di-aryl/heteroaryl methane in which one or both of the aryl/heteroaryl groups is capable of stabilising or facilitating formation of an anion formed on the methylene group between E and R^{1a}. For example, R^{1a} may advantageously be a pyridine group. The starting material (LXI) is reacted with the N-protected bis-2-chloroethylamine (LXII) in the presence of a non-interfering strong base such as sodium hexamethyldisilazide in a polar solvent such as tetrahydrofuran at a reduced temperature (e.g. around 0° C.) to give the N-protected cyclic intermediate (LXIII). The protecting group can be any standard amine-protecting group such as a Boc group. Following cyclisation, the intermediate (LXIII) is coupled to a boronate of the formula (XV) under Suzuki coupling conditions and then deprotected to give the compound of the formula (I).

Compounds of the formula (I) in which the moiety:

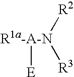

is a group:

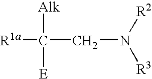

wherein “Alk” is a small alkyl group such as methyl or ethyl can be formed by the synthetic route illustrated in Scheme 10.

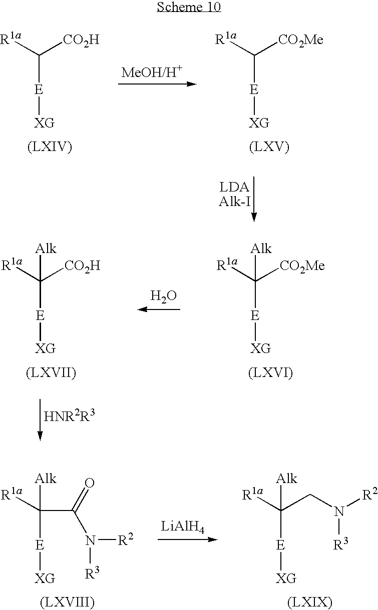

In Scheme 10, a carboxylic acid of the formula (LXIV) is esterified by treatment with methanol in the presence of an acid catalyst such as hydrochloric acid. The ester (LXV) is then reacted with a strong base such as lithium diisopropylamide (LDA) and an alkyl iodide such as methyl iodide at reduced temperature (e.g. between 0° C. and −78° C.). The branched ester (LXVI) is then hydrolysed to the acid (LXVII) and coupled with an amine HNR²R³ under standard amide forming conditions of the type described above. The amide (LXVIII) can then be reduced to the amine (LXIX) using lithium aluminium hydride, and the amine (LXIX) is then reacted with a heteroaryl boronate or boronic acid under Suzuki coupling conditions to give a compound of the formula (I).

Another method of preparing compounds of the formula (I) involves the replacement of the bromine atom in the intermediate of formula (LXX) with a range of heterocyclic ring-precursor groups, and then the conversion of a ring precursor group into a heterocyclic ring.

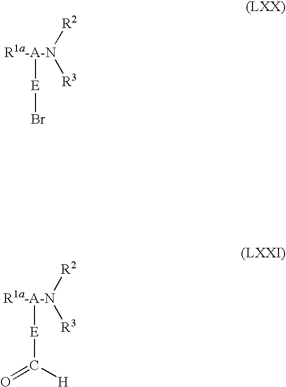

In particular, when the group E is an aryl or heteroaryl group such as a phenyl group, the bromine atom in the compound of formula (LXX) can be converted by well known synthetic methods into, for example, CONH₂, NH₂, COOH, CHO or C(O)CH₃ group, each of which groups may be used for the construction of various heterocyclic ring systems.

By way of example, the bromo-compound of formula (LXX) may be converted to the aldehyde (LXXI) by reacting the bromo-compound with an alkyl lithium such as butyl lithium and then formylating the resulting lithiated intermediate using dimethylformamide. The lithiation step is typically carried out in a dry polar aprotic solvent such as THF at a low temperature (e.g. less than −50° C.

The aldehyde group in the compound (LXXI) can then be converted into a range of heterocyclic groups using chemistry well known to the skilled person. For example, by reacting the aldehyde with tosylmethylisocyanide (tosmic), the aldehyde can be converted into an oxazole ring.

Compounds of the formula (I) wherein TG is

(2) a group:

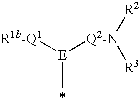

and E is an aryl or heteroaryl group can be prepared by reaction of a compound of the formula (CX) with a compound of the formula (XI) where (CX) and (XI) may be suitably protected and wherein Q¹, Q², E, and R^{1b} to R⁴ are as hereinbefore defined, one of the groups XG and Y is chlorine, bromine or iodine or a trifluoromethanesulphonate (triflate) group, and the other one of the groups X and Y is a boronate residue, for example a boronate ester or boronic acid residue.

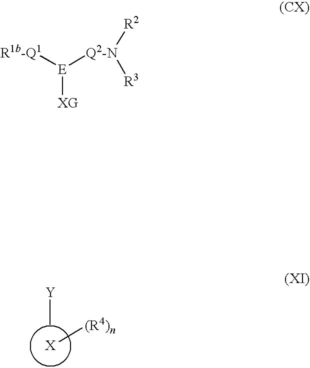

The reaction can be carried out under typical Suzuki Coupling conditions as described above.

An illustrative synthetic route involving a Suzuki coupling step is shown in Scheme 11. In Scheme 11, the bromo compound (CXII) in which E is an aryl or heteroaryl group, is converted to a boronic acid (CXIII) by reaction with an alkyl lithium such as butyl lithium and a borate ester (iPrO)₃B. The reaction is typically carried out in a dry polar solvent such as tetrahydrofuran at a reduced temperature (for example −78° C.).

The resulting boronic acid (CXIII) is then reacted with the N-protected chloro compound (XIV) in the presence of bis(triphenylphosphine)palladium under the conditions described above to give a compound of the formula (CXV).

In Scheme 1, the nitrogen atom in NR²R³ is typically protected with a suitable protecting group of which examples are set out below. One particular protecting group which may be used in the context of a Suzuki coupling for protecting an amino group is the tert-butoxycarbonyl group which can be introduced by reacting the amino group with di-tert-butylcarbonate in the presence of a base such as triethylamine. Removal of the protecting group from a compound of the formula (CXV) can be typically accomplished by methods well known to the skilled person.

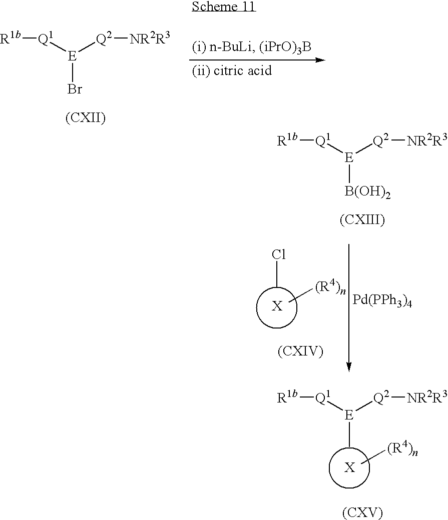

In the preparative procedure outlined above, the coupling of the aryl or heteroaryl group E to the bicyclic group is accomplished by reacting a halo-substituted bicyclic group X with a boronate ester or boronic acid in the presence of a palladium catalyst and base. Boronates suitable for use in preparing compounds of the invention are described above.

Compounds of the formula (I) wherein TG is group (2) and E is a non-aromatic cyclic group and is linked to the bicyclic group by a nitrogen atom can be prepared by the reaction of a compound of the formula (CXIV), in which the ring X is set up to allow nucleophilic displacement of the chlorine atom, with a compound of the formula (CXVII) or a protected derivative thereof, where R^{1b}, Q¹, Q² and NR²R³ are as defined herein and the ring E represents a cyclic group E containing a nucleophilic NH group as a ring member.

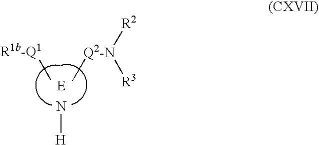

The reaction is typically carried out in a polar solvent such as an alcohol (e.g. ethanol, propanol or n-butanol) at an elevated temperature, for example a temperature in the region from 90° C. to 160° C., optionally in the presence of a non-interfering amine such as triethylamine. The reaction may be carried out in a sealed tube, particularly where the desired reaction temperature exceeds the boiling point of the solvent. When T is N, the reaction is typically carried out at a temperature in the range from about 100° C. to 130° C. but, when T is CH, higher temperatures may be required, for example up to about 160° C., and hence higher boiling solvents such as dimethylformamide may be used. In general, an excess of the nucleophilic amine will be used and/or an additional non-reacting base such as triethylamine will be included in the reaction mixture. Heating of the reaction mixture may be accomplished by normal means or by the use of a microwave heater.

Intermediate compounds of the formula (CXVII) wherein E is a piperidine group, Q¹ is a saturated hydrocarbon linking group and Q¹ and Q² are both linked to the 4-position of the piperidine group can be prepared by the sequence of reactions shown in Scheme 12.

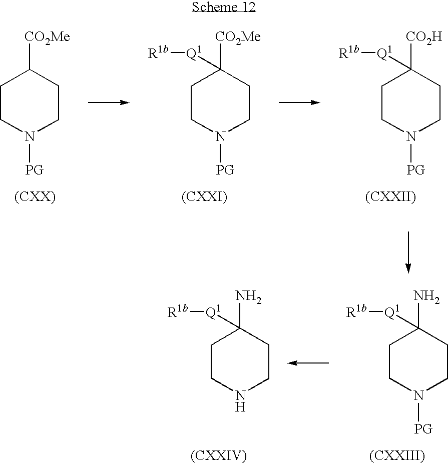

In Scheme 12, 4-methoxycarbonyl-piperidine is first protected (PG=protecting group) in standard fashion, for example by means of a t-butyloxycarbonyl (boc) group by reaction with di-tert-butylcarbonate in the presence of a non-interfering base to give the protected compound (CXX). The protected piperidine carboxymethyl ester (CXX) is then alkylated at the α-position relative to the carbonyl group of the ester by reacting with a strong base such as lithium diisopropylamide (LDA) and a compound of the formula R^{1b}Q¹-Hal where Hal is a halogen, preferably bromine, and Q¹ is a saturated hydrocarbon group. The ester (CXXI) is then hydrolysed to the corresponding carboxylic acid (CXXII) using an alkali metal hydroxide such as sodium hydroxide. The carboxylic acid (CXXII) can be used to prepare a range of different amine intermediates which can, in turn, be converted into compounds of the formula (I). For example, the carboxylic acid can be converted to the acid chloride (e.g. by treatment with oxalyl chloride and optionally a catalytic quantity of DMF, or by treatment of a salt of the acid with oxalyl chloride) and then reacted with sodium azide to form the acid azide (not shown). The acid azide can then be heated to bring about rearrangement in a Curtius reaction (see Advanced Organic Chemistry, 4^{th} edition, by Jerry March, John Wiley & sons, 1992, pages 1091-1092) to give compound (CXXIII) in which the amino group is attached directly to the piperidine ring. The amine (CXXIII) is then deprotected according to standard methods (e.g. using hydrochloric acid in the case of a Boc protecting group) and reacted with a compound of the formula (CXIV) to give a compound of the formula (I).

In an alternative sequence of reactions, the ester (CXXI) can be reduced to the corresponding alcohol which, following deprotection of the piperidine ring nitrogen atom, can be reacted with a compound of the formula (CXXI) to give an alcohol which can be oxidised to the aldehyde using Dess-Martin periodinane (see Dess, D. B.; Martin, J. C. J. Org. Soc., 1983, 48, 4155 and Organic Syntheses, Vol. 77, 141) or tetrapropylammonium perruthenate (TPAP). The resulting aldehyde can be used for a variety of synthetic interconversions such as reductive amination using sodium cyanoborohydride and an amine HNR²R³ to give a compound of the formula (CXVII) in which Q² is CH₂.

The carboxylic acid (CXXII) can also be converted to an amide by reaction with an amine HNR²R³ under conditions suitable for forming an amide bond as described above.

The resulting amide (not shown) can be reduced using a hydride reducing agent such as lithium aluminium hydride in the presence of aluminium chloride to give the corresponding amine.

Compounds of the formula ((CXVII) in which E is a piperidine group, Q¹ is a bond and R^{1b} is an aryl or heteroaryl group R^{1a} can be prepared using the sequence of steps shown in Scheme 13.

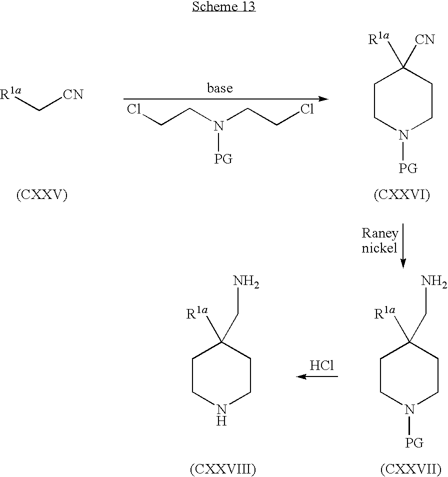

As shown in Scheme 13, the nitrile (CXXV) in which R^{1a} is an aryl or heteroaryl group is reacted with a base and N-protected (PG=protecting group) bis-(2-chloroethyl)amine to give the piperidine nitrile (CXXVI) which can then be reduced to give the amine (CXXVII) using Raney nickel and then deprotected (e.g. using HCl when the protecting group is acid labile) to give amine (CXXVIII). Alternatively, the nitrile (CXXVI) can be reacted with a compound of the formula (CXVI) to give a compound of the formula (I) in which Q² and NR²R³ together form a nitrile group.

Compounds of the formula (I) in which E is a piperidine ring and Q² is a bond and NR²R³ is an amino group can also be prepared by the reaction sequence shown in Scheme 14.

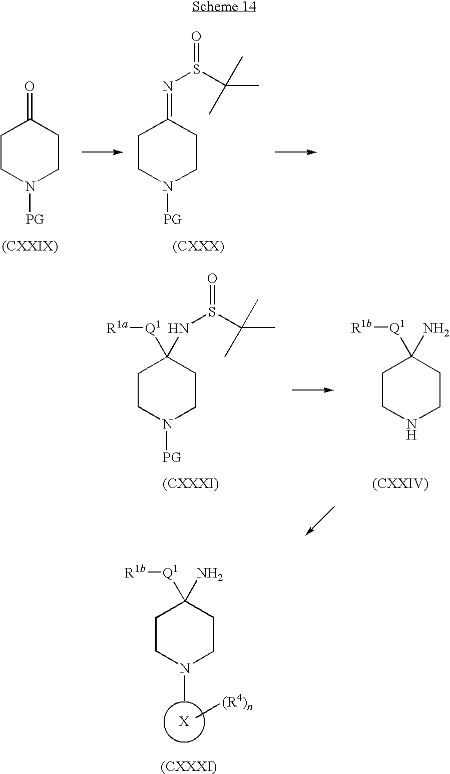

As shown in Scheme 14, a protected 4-piperidone (CXXIX), in which PG is a protecting group such as Boc, is reacted with tert-butylsulphinimide in the presence of titanium tetraethoxide in a dry polar solvent such as THF to give the sulphinimine (CXXX). The reaction is typically carried out with heating, for example to the reflux temperature of the solvent. The sulphinimine (CXXX) is then reacted with an organometallic reagent, for example a Grignard reagent such as an aralkyl or arylmagnesium bromide, suitable for introducing the moiety R^{1a}-Q¹, to give the sulphinamide (CXXXI). The tert-butylsulphinyl group can then be removed by hydrolysis in a hydrochloric acid/dioxane/methanol mixture to give the amine (XXIV). The amine (XXIV) can then be reacted with a chloro-heterocycle (XVI) under the conditions described above to give the product (CXXXI), i.e. a compound of the formula (I) in which E is piperidine Q² is a bond and NR²R³ is an amino group.

The corresponding compound wherein Q² is a bond and NR²R³ is an alkylamino (e.g. methylamino) group can be prepared from the tert-butylsulphinyl intermediate compound (CXXXI) by reaction of the intermediate (CXXXI) with a strong base, e.g. a metal hydride such as sodium hydride, followed by the addition of an alkyl halide such as methyl iodide. The reaction is typically carried out in a polar aprotic solvent such as dimethylformamide at a reduced temperature, for example 0-5° C.

Compounds of the formula (I) where Q¹ contains an amide bond can be prepared from intermediates of the formulae (CXXXII) and (CXXXIII) by reaction with intermediate (XI) above using a Suzuki coupling procedure (when X^{L} is bromine) or by reaction with intermediate (CXIV) (when X^{L} is hydrogen and the group E contains a nucleophilic nitrogen atom) using the methods and conditions described above.

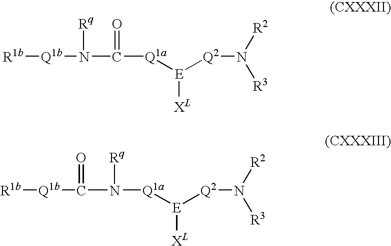

In formulae (CXXXII) and (CXXXIII), Q^{1a} and Q^{1b} are each a bond or a residue of the group Q¹, and X^{L} is hydrogen or halogen such as bromine. For example, Q^{1a} can be a bond and Q^{1b} can be a group CH₂ and vice versa.

The compounds of formulae (CXXXII) and (CXXXIII) can be prepared by reacting together the appropriate carboxylic acid or activated derivative thereof (e.g. acid chloride) and the appropriate amine using the amide-forming conditions described above.

The formation of compounds of the formula (I) wherein the moiety Q¹ contains an amide group is illustrated by the sequence of reactions set out in Scheme 15.

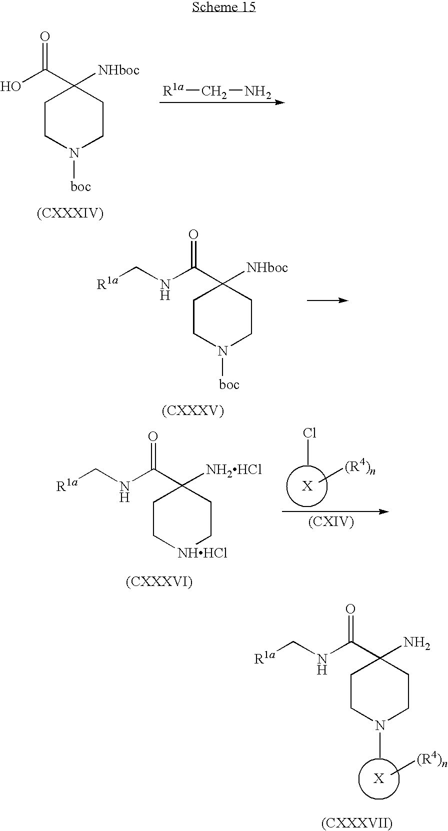

In Scheme 15, the boc-protected piperidine amino acid (CXXXIV) is reacted with the arylamine or heteroarylamine R^{1a}—NH₂ using the amide forming conditions set out above. Thus, for example, the amide-forming reaction can be carried out using HATU (see above) in the presence of a base such as N-ethyldiisopropylamine in a polar solvent such as DMF. The amide (CXXXV) is then deprotected; in this case by treatment with acid to remove the boc group; and then reacted with the bicyclic chloro compound (CXIV) at elevated temperature (e.g. approximately 100° C.) to give the product (CXXXVII). The reaction with the chloro compound is typically carried out in a polar solvent such as a high boiling alcohol (e.g. n-butanol) in the presence of a non-interfering base such as triethylamine.

Compounds of the formula (I) in which Q¹ contains an ether linkage can be prepared in a manner analogous to the methods described above for the compounds in which Q¹ contains an amide bond. The preparation of compounds containing an ether linkage is illustrated by the sequence of reactions set out in Scheme 16.

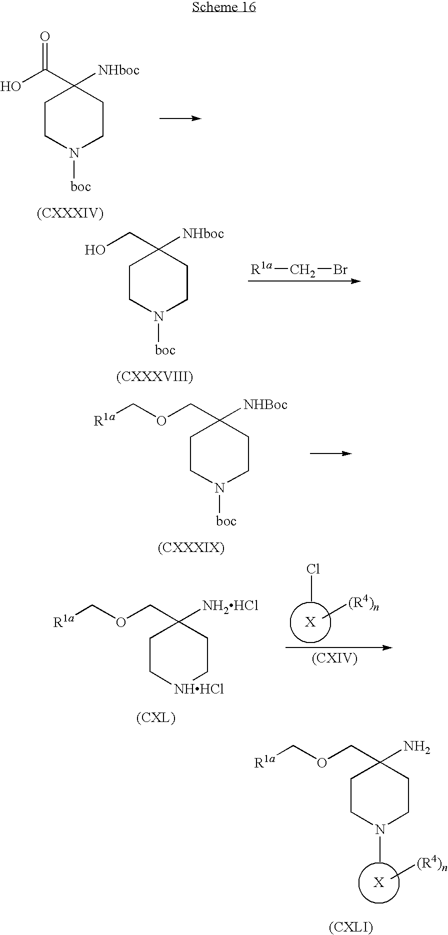

In Scheme 6, the N-protected piperidine amino acid (CXXXIV) is reduced to the corresponding alcohol (CXXXVIII) using a reducing agent such as lithium aluminium hydride in a polar aprotic solvent such as tetrahydrofuran, typically at around room temperature. The alcohol (CXXXVIII) is then treated with a strong base, e.g. a metal hydride such as sodium hydride to form the alcoholate which is then reacted with the arylmethyl- or heteroarylmethyl bromide R^{1a}—CH₂—Br to form the ether (CXXXIX). The ether-forming reaction is typically carried out at a reduced temperature (e.g. approximately 0° C. using an aprotic polar solvent such as DMF. The ether is then deprotected by standard methods and the deprotected ether (CXL) is reacted with the chloro-compound (CXIV) under the conditions described above to give the product (CXLI).

Once formed, many compounds of the formula (I) can be converted into other compounds of the formula (I) using standard functional group interconversions. For example, compounds of the formula (I) in which the NR²R³ forms part of a nitrile group can be reduced to the corresponding amine. Compounds in which NR²R³ is an NH₂ group can be converted to the corresponding alkylamine by reductive alkylation, or to a cyclic group. Compounds wherein R^{1a} contains a halogen atom such as chlorine or bromine can be used to introduce an aryl or heteroaryl group substituent into the R^{1a} group by means of a Suzuki coupling reaction. Further examples of interconversions of one compound of the formula (I) to another compound of the formula (I) can be found in the examples below. Additional examples of functional group interconversions and reagents and conditions for carrying out such conversions can be found in, for example, Advanced Organic Chemistry, by Jerry March, 4^{th} edition, 119, Wiley Interscience, New York, Fiesers' Reagents for Organic Synthesis, Volumes 1-17, John Wiley, edited by Mary Fieser (ISBN: 0-471-58283-2), and Organic Syntheses, Volumes 1-8, John Wiley, edited by Jeremiah P. Freeman (ISBN: 0-471-31192-8).

In many of the reactions described above, it may be necessary to protect one or more groups to prevent reaction from taking place at an undesirable location on the molecule. Examples of protecting groups, and methods of protecting and deprotecting functional groups, can be found in Protective Groups in Organic Synthesis (T. Green and P. Wuts; 3rd Edition; John Wiley and Sons, 1999).

A hydroxy group may be protected, for example, as an ether (—OR) or an ester (—OC(═O)R), for example, as: a t-butyl ether; a benzyl, benzhydryl (diphenylmethyl), or trityl (triphenylmethyl)ether; a trimethylsilyl or t-butyldimethylsilyl ether; or an acetyl ester (—OC(═O)CH₃, —OAc). An aldehyde or ketone group may be protected, for example, as an acetal (R—CH(OR)₂) or ketal (R₂C(OR)₂), respectively, in which the carbonyl group (>C═O) is converted to a diether (>C(OR)₂), by reaction with, for example, a primary alcohol. The aldehyde or ketone group is readily regenerated by hydrolysis using a large excess of water in the presence of acid. An amine group may be protected, for example, as an amide (—NRCO—R) or a urethane (—NRCO—OR), for example, as: a methyl amide (—NHCO—CH₃); a benzyloxy amide (—NHCO—OCH₂C₆H₅, —NH-Cbz); as a t-butoxy amide (—NHCO—OC(CH₃)₃, —NH-Boc); a 2-biphenyl-2-propoxy amide (—NHCO—OC(CH₃)₂C₆H₄C₆H₅, —NH-Bpoc), as a 9-fluorenylmethoxy amide (—NH-Fmoc), as a 6-nitroveratryloxy amide (—NH-Nvoc), as a 2-trimethylsilylethyloxy amide (—NH-Teoc), as a 2,2,2-trichloroethyloxy amide (—NH-Troc), as an allyloxy amide (—NH-Alloc), or as a 2-(phenylsulphonyl)ethyloxy amide (—NH-Psec). Other protecting groups for amines, such as cyclic amines and heterocyclic N—H groups, include toluenesulphonyl (tosyl) and methanesulphonyl (mesyl) groups and benzyl groups such as a para-methoxybenzyl (PMB) group. A carboxylic acid group may be protected as an ester for example, as: an C₁₋₇ alkyl ester (e.g., a methyl ester; a t-butyl ester); a C₁₋₇ haloalkyl ester (e.g., a C₁₋₇ trihaloalkyl ester); a triC₁₋₇ alkylsilyl-C₁₋₇alkyl ester; or a C₅₋₂₀ aryl-C₁₋₇ alkyl ester (e.g., a benzyl ester; a nitrobenzyl ester); or as an amide, for example, as a methyl amide. A thiol group may be protected, for example, as a thioether (—SR), for example, as: a benzyl thioether; an acetamidomethyl ether (—S—CH₂NHC(═O)CH₃).

Many of the chemical intermediates described above are novel and such novel intermediates form a further aspect of the invention.

### Pharmaceutical Formulations

While it is possible for the active compound to be administered alone, it is preferable to present it as a pharmaceutical composition (e.g. formulation) comprising at least one active compound of the invention together with one or more pharmaceutically acceptable carriers, adjuvants, excipients, diluents, fillers, buffers, stabilisers, preservatives, lubricants, or other materials well known to those skilled in the art and optionally other therapeutic or prophylactic agents

Thus, the present invention further provides pharmaceutical compositions, as defined above, and methods of making a pharmaceutical composition comprising admixing at least one active compound, as defined above, together with one or more pharmaceutically acceptable carriers, excipients, buffers, adjuvants, stabilizers, or other materials, as described herein.

The term “pharmaceutically acceptable” as used herein pertains to compounds, materials, compositions, and/or dosage forms which are, within the scope of sound medical judgment, suitable for use in contact with the tissues of a subject (e.g. human) without excessive toxicity, irritation, allergic response, or other problem or complication, commensurate with a reasonable benefit/risk ratio. Each carrier, excipient, etc. must also be “acceptable” in the sense of being compatible with the other ingredients of the formulation.

Pharmaceutical compositions containing compounds of the formula (I) can be formulated in accordance with known techniques, see for example, Remington's Pharmaceutical Sciences, Mack Publishing Company, Easton, Pa., USA.

Accordingly, in a further aspect, the invention provides compounds of the formula (I) and sub-groups thereof as defined herein in the form of pharmaceutical compositions.

The pharmaceutical compositions can be in any form suitable for oral, parenteral, topical, intranasal, ophthalmic, otic, rectal, intra-vaginal, or transdermal administration. Where the compositions are intended for parenteral administration, they can be formulated for intravenous, intramuscular, intraperitoneal, subcutaneous administration or for direct delivery into a target organ or tissue by injection, infusion or other means of delivery. The delivery can be by bolus injection, short term infusion or longer term infusion and can be via passive delivery or through the utilisation of a suitable infusion pump.

Pharmaceutical formulations adapted for parenteral administration include aqueous and non-aqueous sterile injection solutions which may contain anti-oxidants, buffers, bacteriostats, co-solvents, organic solvent mixtures, cyclodextrin complexation agents, emulsifying agents (for forming and stabilizing emulsion formulations), liposome components for forming liposomes, gellable polymers for forming polymeric gels, lyophilisation protectants and combinations of agents for, inter alia, stabilising the active ingredient in a soluble form and rendering the formulation isotonic with the blood of the intended recipient. Pharmaceutical formulations for parenteral administration may also take the form of aqueous and non-aqueous sterile suspensions which may include suspending agents and thickening agents (R. G. Strickly, Solubilizing Excipients in oral and injectable formulations, Pharmaceutical Research, Vol 21(2) 2004, p 201-230).

Liposomes are closed spherical vesicles composed of outer lipid bilayer membranes and an inner aqueous core and with an overall diameter of <100 μm. Depending on the level of hydrophobicity, moderately hydrophobic drugs can be solubilized by liposomes if the drug becomes encapsulated or intercalated within the liposome. Hydrophobic drugs can also be solubilized by liposomes if the drug molecule becomes an integral part of the lipid bilayer membrane, and in this case, the hydrophobic drug is dissolved in the lipid portion of the lipid bilayer.

The formulations may be presented in unit-dose or multi-dose containers, for example sealed ampoules and vials, and may be stored in a freeze-dried (lyophilised) condition requiring only the addition of the sterile liquid carrier, for example water for injections, immediately prior to use.

The pharmaceutical formulation can be prepared by lyophilising a compound of formula (I), or sub-groups thereof. Lyophilisation refers to the procedure of freeze-drying a composition. Freeze-drying and lyophilisation are therefore used herein as synonyms.

Extemporaneous injection solutions and suspensions may be prepared from sterile powders, granules and tablets.

Pharmaceutical compositions of the present invention for parenteral injection can also comprise pharmaceutically acceptable sterile aqueous or non-aqueous solutions, dispersions, suspensions or emulsions as well as sterile powders for reconstitution into sterile injectable solutions or dispersions just prior to use. Examples of suitable aqueous and nonaqueous carriers, diluents, solvents or vehicles include water, ethanol, polyols (such as glycerol, propylene glycol, polyethylene glycol, and the like), carboxymethylcellulose and suitable mixtures thereof, vegetable oils (such as olive oil), and injectable organic esters such as ethyl oleate. Proper fluidity can be maintained, for example, by the use of coating materials such as lecithin, by the maintenance of the required particle size in the case of dispersions, and by the use of surfactants.

The compositions of the present invention may also contain adjuvants such as preservatives, wetting agents, emulsifying agents, and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents such as sugars, sodium chloride, and the like. Prolonged absorption of the injectable pharmaceutical form may be brought about by the inclusion of agents which delay absorption such as aluminum monostearate and gelatin.

In one preferred embodiment of the invention, the pharmaceutical composition is in a form suitable for i.v. administration, for example by injection or infusion. For intravenous administration, the solution can be dosed as is, or can be injected into an infusion bag (containing a pharmaceutically acceptable excipient, such as 0.9% saline or 5% dextrose), before administration.

In another preferred embodiment, the pharmaceutical composition is in a form suitable for sub-cutaneous (s.c.) administration.

Pharmaceutical dosage forms suitable for oral administration include tablets, capsules, caplets, pills, lozenges, syrups, solutions, powders, granules, elixirs and suspensions, sublingual tablets, wafers or patches and buccal patches.

Thus, tablet compositions can contain a unit dosage of active compound together with an inert diluent or carrier such as a sugar or sugar alcohol, eg; lactose, sucrose, sorbitol or mannitol; and/or a non-sugar derived diluent such as sodium carbonate, calcium phosphate, calcium carbonate, or a cellulose or derivative thereof such as methyl cellulose, ethyl cellulose, hydroxypropyl methyl cellulose, and starches such as corn starch. Tablets may also contain such standard ingredients as binding and granulating agents such as polyvinylpyrrolidone, disintegrants (e.g. swellable crosslinked polymers such as crosslinked carboxymethylcellulose), lubricating agents (e.g. stearates), preservatives (e.g. parabens), antioxidants (e.g. BHT), buffering agents (for example phosphate or citrate buffers), and effervescent agents such as citrate/bicarbonate mixtures. Such excipients are well known and do not need to be discussed in detail here.

Capsule formulations may be of the hard gelatin or soft gelatin variety and can contain the active component in solid, semi-solid, or liquid form. Gelatin capsules can be formed from animal gelatin or synthetic or plant derived equivalents thereof.

The solid dosage forms (eg; tablets, capsules etc.) can be coated or un-coated, but typically have a coating, for example a protective film coating (e.g. a wax or varnish) or a release controlling coating. The coating (e.g. a Eudragit™ type polymer) can be designed to release the active component at a desired location within the gastro-intestinal tract. Thus, the coating can be selected so as to degrade under certain pH conditions within the gastrointestinal tract, thereby selectively release the compound in the stomach or in the ileum or duodenum.

Instead of, or in addition to, a coating, the drug can be presented in a solid matrix comprising a release controlling agent, for example a release delaying agent which may be adapted to selectively release the compound under conditions of varying acidity or alkalinity in the gastrointestinal tract. Alternatively, the matrix material or release retarding coating can take the form of an erodible polymer (e.g. a maleic anhydride polymer) which is substantially continuously eroded as the dosage form passes through the gastrointestinal tract. As a further alternative, the active compound can be formulated in a delivery system that provides osmotic control of the release of the compound. Osmotic release and other delayed release or sustained release formulations may be prepared in accordance with methods well known to those skilled in the art.

The pharmaceutical compositions comprise from approximately 1% to approximately 95%, preferably from approximately 20% to approximately 90%, active ingredient. Pharmaceutical compositions according to the invention may be, for example, in unit dose form, such as in the form of ampoules, vials, suppositories, dragées, tablets or capsules.

Pharmaceutical compositions for oral administration can be obtained by combining the active ingredient with solid carriers, if desired granulating a resulting mixture, and processing the mixture, if desired or necessary, after the addition of appropriate excipients, into tablets, dragee cores or capsules. It is also possible for them to be incorporated into plastics carriers that allow the active ingredients to diffuse or be released in measured amounts.

The compounds of the invention can also be formulated as solid dispersions. Solid dispersions are homogeneous extremely fine disperse phases of two or more solids. Solid solutions (molecularly disperse systems), one type of solid dispersion, are well known for use in pharmaceutical technology (see (Chiou and Riegelman, J. Pharm. Sci., 60, 1281-1300 (1971)) and are useful in increasing dissolution rates and increasing the bioavailability of poorly water-soluble drugs.

This invention also provides solid dosage forms comprising the solid solution described above. Solid dosage forms include tablets, capsules and chewable tablets. Known excipients can be blended with the solid solution to provide the desired dosage form. For example, a capsule can contain the solid solution blended with (a) a disintegrant and a lubricant, or (b) a disintegrant, a lubricant and a surfactant. A tablet can contain the solid solution blended with at least one disintegrant, a lubricant, a surfactant, and a glidant. The chewable tablet can contain the solid solution blended with a bulking agent, a lubricant, and if desired an additional sweetening agent (such as an artificial sweetener), and suitable flavours.

The pharmaceutical formulations may be presented to a patient in “patient packs” containing an entire course of treatment in a single package, usually a blister pack. Patient packs have an advantage over traditional prescriptions, where a pharmacist divides a patient's supply of a pharmaceutical from a bulk supply, in that the patient always has access to the package insert contained in the patient pack, normally missing in patient prescriptions. The inclusion of a package insert has been shown to improve patient compliance with the physician's instructions.

Compositions for topical use include ointments, creams, sprays, patches, gels, liquid drops and inserts (for example intraocular inserts). Such compositions can be formulated in accordance with known methods.

Examples of formulations for rectal or intra-vaginal administration include pessaries and suppositories which may be, for example, formed from a shaped moldable or waxy material containing the active compound.

Compositions for administration by inhalation may take the form of inhalable powder compositions or liquid or powder sprays, and can be administrated in standard form using powder inhaler devices or aerosol dispensing devices. Such devices are well known. For administration by inhalation, the powdered formulations typically comprise the active compound together with an inert solid powdered diluent such as lactose.

The compounds of the formula (I) will generally be presented in unit dosage form and, as such, will typically contain sufficient compound to provide a desired level of biological activity. For example, a formulation may contain from 1 nanogram to 2 grams of active ingredient, e.g. from 1 nanogram to 2 milligrams of active ingredient. Within this range, particular sub-ranges of compound are 0.1 milligrams to 2 grams of active ingredient (more usually from 10 milligrams to 1 gram, e.g. 50 milligrams to 500 milligrams), or 1 microgram to 20 milligrams (for example 1 microgram to 10 milligrams, e.g. 0.1 milligrams to 2 milligrams of active ingredient).

For oral compositions, a unit dosage form may contain from 1 milligram to 2 grams, more typically 10 milligrams to 1 gram, for example 50 milligrams to 1 gram, e.g. 100 milligrams to 1 gram, of active compound.

The active compound will be administered to a patient in need thereof (for example a human or animal patient) in an amount sufficient to achieve the desired therapeutic effect.

### Protein Kinase Inhibitory Activity

The activity of the compounds of the invention as inhibitors of protein kinase A and protein kinase B can be measured using the assays set forth in the examples below and the level of activity exhibited by a given compound can be defined in terms of the IC50 value. Preferred compounds of the present invention are compounds having an IC₅₀ value of less than 20 μM, more preferably less than 10 μM, against protein kinase B.

### Therapeutic Uses

### Prevention or Treatment of Proliferative Disorders

The compounds of the formula (I) are inhibitors of protein kinase A and protein kinase B. As such, they are expected to be useful in providing a means of preventing the growth of or inducing apoptosis of neoplasias. It is therefore anticipated that the compounds will prove useful in treating or preventing proliferative disorders such as cancers. In particular tumours with deletions or inactivating mutations in PTEN or loss of PTEN expression or rearrangements in the (T-cell lymphocyte) TCL-1 gene may be particularly sensitive to PKB inhibitors. Tumours which have other abnormalities leading to an upregulated PKB pathway signal may also be particularly sensitive to inhibitors of PKB. Examples of such abnormalities include but are not limited to overexpression of one or more PI3K subunits, over-expression of one or more PKB isoforms, or mutations in PI3K, PDK1, or PKB which lead to an increase in the basal activity of the enzyme in question, or upregulation or overexpression or mutational activation of a growth factor receptor such as a growth factor selected from the epidermal growth factor receptor (EGFR), fibroblast growth factor receptor (FGFR), platelet derived growth factor receptor (PDGFR), insulin-like growth factor 1 receptor (IGF-1R) and vascular endothelial growth factor receptor (VEGFR) families.

It is also envisaged that the compounds of the invention will be useful in treating other conditions which result from disorders in proliferation or survival such as viral infections, and neurodegenerative diseases for example. PKB plays an important role in maintaining the survival of immune cells during an immune response and therefore PKB inhibitors could be particularly beneficial in immune disorders including autoimmune conditions.

Therefore, PKB inhibitors could be useful in the treatment of diseases in which there is a disorder of proliferation, apoptosis or differentiation.

PKB inhibitors may also be useful in diseases resulting from insulin resistance and insensitivity, and the disruption of glucose, energy and fat storage such as metabolic disease and obesity.

Examples of cancers which may be inhibited include, but are not limited to, a carcinoma, for example a carcinoma of the bladder, breast, colon (e.g. colorectal carcinomas such as colon adenocarcinoma and colon adenoma), kidney, epidermal, liver, lung, for example adenocarcinoma, small cell lung cancer and non-small cell lung carcinomas, esophagus, gall bladder, ovary, pancreas e.g. exocrine pancreatic carcinoma, stomach, cervix, endometrium, thyroid, prostate, or skin, for example squamous cell carcinoma; a hematopoetic malignancy for example acute myeloid leukaemia, acute promyelocytic leukaemia, acute lymphoblastic leukaemia, chronic myeloid leukaemia, chronic lymphocytic leukaemia and other B-cell lymphoproliferative diseases, myelodysplastic syndrome, T-cell lymphoproliferative diseases including those derived from Natural Killer cells, Non-Hodgkin's lymphoma and Hodgkin's disease; Bortezomib sensitive and refractory multiple myeloma; hematopoetic diseases of abnormal cell proliferation whether pre malignant or stable such as myeloproliferative diseases including polycythemia vera, essential thrombocythemia and primary myelofibrosis; hairy cell lymphoma, or Burkett's lymphoma; a hematopoietic tumour of myeloid lineage, for example acute and chronic myelogenous leukaemias, myelodysplastic syndrome, or promyelocytic leukaemia; thyroid follicular cancer; a tumour of mesenchymal origin, for example fibrosarcoma or habdomyosarcoma; a tumour of the central or peripheral nervous system, for example astrocytoma, neuroblastoma, glioma or schwannoma; melanoma; seminoma; teratocarcinoma; osteosarcoma; xenoderoma pigmentosum; keratoctanthoma; thyroid follicular cancer; or Kaposi's sarcoma.

Thus, in the pharmaceutical compositions, uses or methods of this invention for treating a disease or condition comprising abnormal cell growth, the disease or condition comprising abnormal cell growth in one embodiment is a cancer.

Particular subsets of cancers include breast cancer, ovarian cancer, colon cancer, prostate cancer, oesophageal cancer, squamous cancer and non-small cell lung carcinomas.

A further subset of cancers includes breast cancer, ovarian cancer, prostate cancer, endometrial cancer and glioma.

It is also possible that some protein kinase B inhibitors can be used in combination with other anticancer agents. For example, it may be beneficial to combine of an inhibitor that induces apoptosis with another agent which acts via a different mechanism to regulate cell growth thus treating two of the characteristic features of cancer development. Examples of such combinations are set out below.

### Immune Disorders

Immune disorders for which PKA and PKB inhibitors may be beneficial include but are not limited to autoimmune conditions and chronic inflammatory diseases, for example systemic lupus erythematosus, autoimmune mediated glomerulonephritis, rheumatoid arthritis, psoriasis, inflammatory bowel disease, and autoimmune diabetes mellitus, Eczema hypersensitivity reactions, asthma, COPD, rhinitis, and upper respiratory tract disease.

### Other Therapeutic Uses

PKB plays a role in apoptosis, proliferation, differentiation and therefore PKB inhibitors could also be useful in the treatment of the following diseases other than cancer and those associated with immune dysfunction; viral infections, for example herpes virus, pox virus, Epstein-Barr virus, Sindbis virus, adenovirus, HIV, HPV, HCV and HCMV; prevention of AIDS development in HIV-infected individuals; cardiovascular diseases for example cardiac hypertrophy, restenosis, atherosclerosis; neurodegenerative disorders, for example Alzheimer's disease, AIDS-related dementia, Parkinson's disease, amyotropic lateral sclerosis, retinitis pigmentosa, spinal muscular atrophy and cerebellar degeneration; glomerulonephritis; myelodysplastic syndromes, ischemic injury associated myocardial infarctions, stroke and reperfusion injury, degenerative diseases of the musculoskeletal system, for example, osteoporosis and arthritis, aspirin-sensitive rhinosinusitis, cystic fibrosis, multiple sclerosis, kidney diseases.

### Methods of Treatment

It is envisaged that the compounds of the formula (I) and sub-groups thereof as defined herein will be useful in the prophylaxis or treatment of a range of disease states or conditions mediated by protein kinase A and/or protein kinase B. Examples of such disease states and conditions are set out above.

The compounds are generally administered to a subject in need of such administration, for example a human or animal patient, preferably a human.

The compounds will typically be administered in amounts that are therapeutically or prophylactically useful and which generally are non-toxic. However, in certain situations (for example in the case of life threatening diseases), the benefits of administering a compound of the formula (I) may outweigh the disadvantages of any toxic effects or side effects, in which case it may be considered desirable to administer compounds in amounts that are associated with a degree of toxicity.

The compounds may be administered over a prolonged term to maintain beneficial therapeutic effects or may be administered for a short period only. Alternatively they may be administered in a pulsatile or continuous manner.

A typical daily dose of the compound of formula (I) can be in the range from 100 picograms to 100 milligrams per kilogram of body weight, more typically 5 nanograms to 25 milligrams per kilogram of bodyweight, and more usually 10 nanograms to 15 milligrams per kilogram (e.g. 10 nanograms to 10 milligrams, and more typically 1 microgram per kilogram to 20 milligrams per kilogram, for example 1 microgram to 10 milligrams per kilogram) per kilogram of bodyweight although higher or lower doses may be administered where required. The compound of the formula (I) can be administered on a daily basis or on a repeat basis every 2, or 3, or 4, or 5, or 6, or 7, or 10 or 14, or 21, or 28 days for example.

The compounds of the invention may be administered orally in a range of doses, for example 1 to 1500 mg, 2 to 800 mg, or 5 to 500 mg, e.g. 2 to 200 mg or 10 to 1000 mg, particular examples of doses including 10, 20, 50 and 80 mg. The compound may be administered once or more than once each day. The compound can be administered continuously (i.e. taken every day without a break for the duration of the treatment regimen). Alternatively, the compound can be administered intermittently, i.e. taken continuously for a given period such as a week, then discontinued for a period such as a week and then taken continuously for another period such as a week and so on throughout the duration of the treatment regimen. Examples of treatment regimens involving intermittent administration include regimens wherein administration is in cycles of one week on, one week off; or two weeks on, one week off; or three weeks on, one week off; or two weeks on, two weeks off; or four weeks on two weeks off; or one week on three weeks off—for one or more cycles, e.g. 2, 3, 4, 5, 6, 7, 8, 9 or 10 or more cycles.

In one particular dosing schedule, a patient will be given an infusion of a compound of the formula (I) for periods of one hour daily for up to ten days in particular up to five days for one week, and the treatment repeated at a desired interval such as two to four weeks, in particular every three weeks.

More particularly, a patient may be given an infusion of a compound of the formula (I) for periods of one hour daily for 5 days and the treatment repeated every three weeks.

In another particular dosing schedule, a patient is given an infusion over 30 minutes to 1 hour followed by maintenance infusions of variable duration, for example 1 to 5 hours, e.g. 3 hours.

In a further particular dosing schedule, a patient is given a continuous infusion for a period of 12 hours to 5 days, an in particular a continuous infusion of 24 hours to 72 hours.

Ultimately, however, the quantity of compound administered and the type of composition used will be commensurate with the nature of the disease or physiological condition being treated and will be at the discretion of the physician.

The compounds as defined herein can be administered as the sole therapeutic agent or they can be administered in combination therapy with one of more other compounds for treatment of a particular disease state, for example a neoplastic disease such as a cancer as hereinbefore defined. Examples of other therapeutic agents or treatments that may be administered together (whether concurrently or at different time intervals) with the compounds of the formula (I) include but are not limited to:
Topoisomerase I inhibitors
Antimetabolites
Tubulin targeting agents
DNA binder and topoisomerase II inhibitors
Alkylating Agents
Monoclonal Antibodies.
Anti-Hormones
Signal Transduction Inhibitors
Proteasome Inhibitors
DNA methyl transferases
Cytokines and retinoids
Chromatin targeted therapies
Radiotherapy, and,
Other therapeutic or prophylactic agents; for example agents that reduce or alleviate some of the side effects associated with chemotherapy. Particular examples of such agents include anti-emetic agents and agents that prevent or decrease the duration of chemotherapy-associated neutropenia and prevent complications that arise from reduced levels of red blood cells or white blood cells, for example erythropoietin (EPO), granulocyte macrophage-colony stimulating factor (GM-CSF), and granulocyte-colony stimulating factor (G-CSF). Also included are agents that inhibit bone resorption such as bisphosphonate agents e.g. zoledronate, pamidronate and ibandronate, agents that suppress inflammatory responses (such as dexamethazone, prednisone, and prednisolone) and agents used to reduce blood levels of growth hormone and IGF-I in acromegaly patients such as synthetic forms of the brain hormone somatostatin, which includes octreotide acetate which is a long-acting octapeptide with pharmacologic properties mimicking those of the natural hormone somatostatin. Further included are agents such as leucovorin, which is used as an antidote to drugs that decrease levels of folic acid, or folinic acid it self and agents such as megestrol acetate which can be used for the treatment of side-effects including oedema and thromboembolic episodes.

Each of the compounds present in the combinations of the invention may be given in individually varying dose schedules and via different routes.

Where the compound of the formula (I) is administered in combination therapy with one, two, three, four or more other therapeutic agents (preferably one or two, more preferably one), the compounds can be administered simultaneously or sequentially. When administered sequentially, they can be administered at closely spaced intervals (for example over a period of 5-10 minutes) or at longer intervals (for example 1, 2, 3, 4 or more hours apart, or even longer periods apart where required), the precise dosage regimen being commensurate with the properties of the therapeutic agent(s).

The compounds of the invention may also be administered in conjunction with non-chemotherapeutic treatments such as radiotherapy, photodynamic therapy, gene therapy; surgery and controlled diets.

For use in combination therapy with another chemotherapeutic agent, the compound of the formula (I) and one, two, three, four or more other therapeutic agents can be, for example, formulated together in a dosage form containing two, three, four or more therapeutic agents. In an alternative, the individual therapeutic agents may be formulated separately and presented together in the form of a kit, optionally with instructions for their use.

A person skilled in the art would know through his or her common general knowledge the dosing regimes and combination therapies to use.

### Methods of Diagnosis

Prior to administration of a compound of the formula (I), a patient may be screened to determine whether a disease or condition from which the patient is or may be suffering is one which would be susceptible to treatment with a compound having activity against protein kinase A and/or protein kinase B.

For example, a biological sample taken from a patient may be analysed to determine whether a condition or disease, such as cancer, that the patient is or may be suffering from is one which is characterised by a genetic abnormality or abnormal protein expression which leads to up-regulation of PKA and/or PKB or to sensitisation of a pathway to normal PKA and/or PKB activity, or to upregulation of a signal transduction component upstream of PKA and/or PKB such as, in the case of PKB, PI3K, GF receptor and PDK 1 & 2.

Alternatively, a biological sample taken from a patient may be analysed for loss of a negative regulator or suppressor of the PKB pathway such as PTEN. In the present context, the term “loss” embraces the deletion of a gene encoding the regulator or suppressor, the truncation of the gene (for example by mutation), the truncation of the transcribed product of the gene, or the inactivation of the transcribed product (e.g. by point mutation) or sequestration by another gene product.

The term up-regulation includes elevated expression or over-expression, including gene amplification (i.e. multiple gene copies) and increased expression by a transcriptional effect, and hyperactivity and activation, including activation by mutations. Thus, the patient may be subjected to a diagnostic test to detect a marker characteristic of up-regulation of PKA and/or PKB. The term diagnosis includes screening. By marker we include genetic markers including, for example, the measurement of DNA composition to identify mutations of PKA and/or PKB. The term marker also includes markers which are characteristic of up regulation of PKA and/or PKB, including enzyme activity, enzyme levels, enzyme state (e.g. phosphorylated or not) and mRNA levels of the aforementioned proteins.

The above diagnostic tests and screens are typically conducted on a biological sample selected from tumour biopsy samples, blood samples (isolation and enrichment of shed tumour cells), stool biopsies, sputum, chromosome analysis, pleural fluid, peritoneal fluid, or urine.

Identification of an individual carrying a mutation in PKA and/or PKB or a rearrangement of TCL-1 or loss of PTEN expression may mean that the patient would be particularly suitable for treatment with a PKA and/or PKB inhibitor. Tumours may preferentially be screened for presence of a PKA and/or PKB variant prior to treatment. The screening process will typically involve direct sequencing, oligonucleotide microarray analysis, or a mutant specific antibody.

Methods of identification and analysis of mutations and up-regulation of proteins are known to a person skilled in the art. Screening methods could include, but are not limited to, standard methods such as reverse-transcriptase polymerase chain reaction (RT-PCR) or in-situ hybridisation.

In screening by RT-PCR, the level of mRNA in the tumour is assessed by creating a cDNA copy of the mRNA followed by amplification of the cDNA by PCR. Methods of PCR amplification, the selection of primers, and conditions for amplification, are known to a person skilled in the art. Nucleic acid manipulations and PCR are carried out by standard methods, as described for example in Ausubel, F. M. et al., eds. Current Protocols in Molecular Biology, 2004, John Wiley & Sons Inc., or Innis, M. A. et-al., eds. PCR Protocols: a guide to methods and applications, 1990, Academic Press, San Diego. Reactions and manipulations involving nucleic acid techniques are also described in Sambrook et al., 2001, 3^{rd} Ed, Molecular Cloning: A Laboratory Manual, Cold Spring Harbor Laboratory Press. Alternatively a commercially available kit for RT-PCR (for example Roche Molecular Biochemicals) may be used, or methodology as set forth in U.S. Pat. Nos. 4,666,828; 4,683,202; 4,801,531; 5,192,659, 5,272,057, 5,882,864, and 6,218,529 and incorporated herein by reference.

An example of an in-situ hybridisation technique for assessing mRNA expression would be fluorescence in-situ hybridisation (FISH) (see Angerer, 1987 Meth. Enzymol., 152: 649).

Generally, in situ hybridization comprises the following major steps: (1) fixation of tissue to be analyzed; (2) prehybridization treatment of the sample to increase accessibility of target nucleic acid, and to reduce nonspecific binding; (3) hybridization of the mixture of nucleic acids to the nucleic acid in the biological structure or tissue; (4) post-hybridization washes to remove nucleic acid fragments not bound in the hybridization, and (5) detection of the hybridized nucleic acid fragments. The probes used in such applications are typically labeled, for example, with radioisotopes or fluorescent reporters. Preferred probes are sufficiently long, for example, from about 50, 100, or 200 nucleotides to about 1000 or more nucleotides, to enable specific hybridization with the target nucleic acid(s) under stringent conditions. Standard methods for carrying out FISH are described in Ausubel, F. M. et al., eds. Current Protocols in Molecular Biology, 2004, John Wiley & Sons Inc and Fluorescence In Situ Hybridization: Technical Overview by John M. S. Bartlett in Molecular Diagnosis of Cancer, Methods and Protocols, 2nd ed.; ISBN: 1-59259-760-2; March 2004, pps. 077-088; Series: Methods in Molecular Medicine.

Alternatively, the protein products expressed from the mRNAs may be assayed by immunohistochemistry of tumour samples, solid phase immunoassay with microtitre plates, Western blotting, 2-dimensional SDS-polyacrylamide gel electrophoresis, ELISA, flow cytometry and other methods known in the art for detection of specific proteins. Detection methods would include the use of site specific antibodies. The skilled person will recognize that all such well-known techniques for detection of upregulation of PKB, or detection of PKB variants could be applicable in the present case.

Therefore all of these techniques could also be used to identify tumours particularly suitable for treatment with PKA and/or PKB inhibitors.

For example, as stated above, PKB beta has been found to be upregulated in 10-40% of ovarian and pancreatic cancers (Bellacosa et al 1995, Int. J. Cancer 64, 280-285; Cheng et al 1996, PNAS 93, 3636-3641; Yuan et al 2000, Oncogene 19, 2324-2330). Therefore it is envisaged that PKB inhibitors, and in particular inhibitors of PKB beta, may be used to treat ovarian and pancreatic cancers.

PKB alpha is amplified in human gastric, prostate and breast cancer (Staal 1987, PNAS 84, 5034-5037; Sun et al 2001, Am. J. Pathol. 159, 431-437). Therefore it is envisaged that PKB inhibitors, and in particular inhibitors of PKB alpha, may be used to treat human gastric, prostate and breast cancer.

Increased PKB gamma activity has been observed in steroid independent breast and prostate cell lines (Nakatani et al 1999, J. Biol. Chem. 274, 21528-21532). Therefore it is envisaged that PKB inhibitors, and in particular inhibitors of PKB gamma, may be used to treat steroid independent breast and prostate cancers.

### EXPERIMENTAL

The invention will now be illustrated, but not limited, by reference to the specific embodiments described in the following procedures and examples.

The starting materials for each of the procedures described below are commercially available unless otherwise specified.

In the examples, the compounds prepared were characterised by liquid chromatography, mass spectroscopy and ¹H nuclear magnetic resonance spectroscopy using the systems and operating conditions set out below.

Proton magnetic resonance (¹H NMR) spectra were recorded on a Bruker AV400 instrument operating at 400.13 MHz, in Me-d₃-OD at 27 C, unless otherwise stated and are reported as follows: chemical shift 6/ppm (number of protons, multiplicity where s=singlet, d=doublet, t=triplet, q=quartet, m=multiplet, br=broad). The residual protic solvent MeOH (6H=3.31 ppm) was used as the internal reference.

For the mass spectra, where chlorine is present, the mass quoted for the compound is for ³⁵Cl.

In each of the examples, where the compounds are isolated or formed as the free base, they can be converted into a salt form such as an acetic acid or hydrochloric acid salt. Conversely, where the compounds are isolated or formed as a salt, the salt can be converted into the corresponding free base by methods well known to the skilled person, and then optionally converted to another salt.

A number of liquid chromatography systems were used and these are described below.

LCT System 1

| | |
|---|---|
| HPLC System: | Waters Alliance 2795 Separations Module |
| Mass Spec Detector: | Waters/Micromass LCT |
| UV Detector: | Waters 2487 Dual λ Absorbance Detector |

Polar Analytical conditions:

| | |
|---|---|
| Eluent A: | Methanol |
| Eluent B: | 0.1% Formic Acid in Water |

Gradient:

| | | |
|---|---|---|
| Time (mins) | A | B |
| 0 | 10 | 90 |
| 0.5 | 10 | 90 |
| 6.5 | 90 | 10 |
| 10 | 90 | 10 |
| 10.5 | 10 | 90 |
| 15 | 10 | 90 |

| | |
|---|---|
| Flow: | 1.0 ml/min |
| Column: | Supelco DISCOVERY C₁₈ |
| | 5 cm × 4.6 mm i.d., 5 μm |

MS conditions:

| | |
|---|---|
| Capillary voltage: | 3500 v (+ve ESI), 3000 v (−ve ESI) |
| Cone voltage: | 40 v (+ve ESI), 50 v (−ve ESI) |
| Source Temperature: | 100° C. |
| Scan Range: | 50-1000 amu |
| Ionisation Mode: | +ve/−ve electrospray ESI (Lockspray ™) |

LCT System 2

| | |
|---|---|
| HPLC System: | Waters Alliance 2795 Separations Module |
| Mass Spec Detector: | Waters/Micromass LCT |
| UV Detector: | Waters 2487 Dual λ Absorbance Detector |

Analytical conditions:

| | |
|---|---|
| Eluent A: | Methanol |
| Eluent B: | 0.1% Formic Acid in Water |

Gradient:

| | | |
|---|---|---|
| Time (mins) | A | B |
| 0 | 10 | 90 |
| 0.6 | 10 | 90 |
| 1.0 | 20 | 80 |
| 7.5 | 90 | 10 |
| 9 | 90 | 10 |
| 9.5 | 10 | 90 |
| 10 | 10 | 90 |

| | |
|---|---|
| Flow: | 1 ml/min |
| Column: | Supelco DISCOVERY C₁₈ |
| | 5 cm × 4.6 mm i.d., 5 μm |

MS conditions:

| | |
|---|---|
| Capillary voltage: | 3500 v (+ve ESI), 3000 v (−ve ESI) |
| Cone voltage: | 40 v (+ve ESI), 50 v (−ve ESI) |
| Source Temperature: | 100° C. |
| Scan Range: | 50-1000 amu |
| Ionisation Mode: | +ve/−ve electrospray ESI (Lockspray ™) |

In the examples below, the following key is used to identify the LCMS conditions
LCT1 LCT System 1-polar analytical conditions
LCT2 LCT System 2-polar analytical conditions

### General Procedures

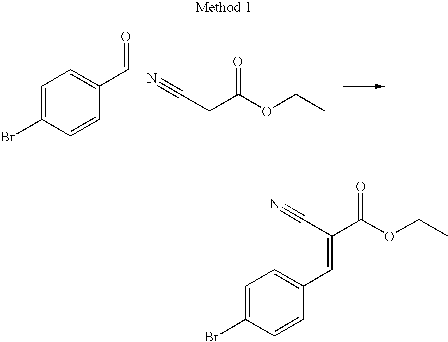

To 4-bromobenzaldehyde (3 g, 16.21 mmol) and ethyl cyanoacetate (1.9 ml, 17.84 mmol, 1.1 equiv.) in toluene was added piperidine (27 μl) and the reaction mixture was refluxed for 1 hour with a Dean-Stark separator. The solvent was removed under reduced pressure, and the residue was triturated with warm ethyl acetate and filtered to yield the desired product as a yellow solid

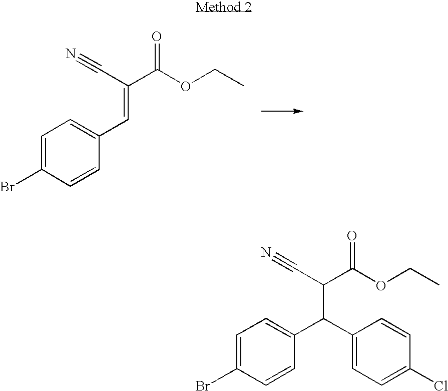

A solution of 3-(4-bromo-phenyl)-2-cyano-acrylic acid ethyl ester (1.5 g, 5.36 mmol) in dry toluene (12 ml) was added dropwise to 4-chlorophenylmagnesium bromide (0.5 M solution in tetrahydrofuran, 6.96 ml, 6.96 mmol, 1.3 equiv.) at 0° C. The reaction mixture was heated to 85° C. for 3 hours, poured onto ice, acidified with 1N HCl and extracted with ethyl acetate. The organic layer was separated, dried (MgSO₄), filtered and concentrated, and the crude product was purified over flash silica chromatography eluting with petroleum ether to ethyl acetate/petroleum ether (5:95) to afford the desired product.

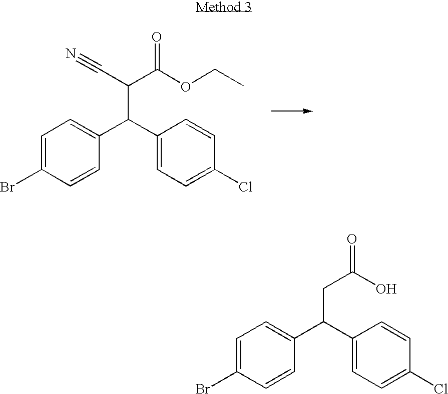

A mixture of 3-(4-bromo-phenyl)-3-(4-chloro-phenyl)-2-cyano-propionic acid ethyl ester (1.91, 4.87 mmol), acetic acid (10 ml), concentrated sulphuric acid (5 ml) and water (5 ml) were refluxed for 2 hours. Reaction mixture was poured into iced water and extracted with ethyl acetate. The organic layer was separated, dried (MgSO₄), filtered and concentrated, the crude product was purified over flash silica chromatography eluting with ethyl acetate/petroleum ether (1:1) to afford the desired product.

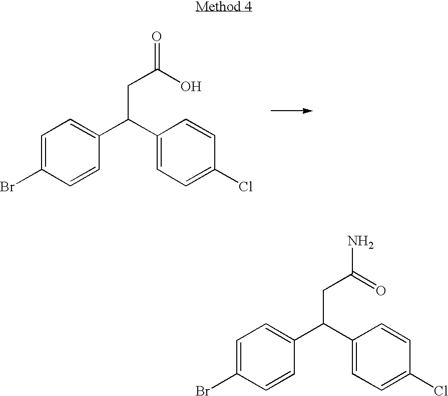

A mixture of 3-(4-bromo-phenyl)-3-(4-chloro-phenyl)-propionic acid (0.25 g, 0.74 mmol) and 1-hydroxybenatriazole (0.12 g, 0.88 mmol) in dichloromethane (3 ml) was stirred for 15 minutes before addition of ammonia (2N solution in methanol, 0.74 ml, 1.47 mmol, 2.0 equiv.) and 1-(3-dimethylaminopropyl)-ethylcarbodiimide hydrochloride (0.17 g, 0.88 mmol, 1.2 equiv). The reaction mixture was stirred for 16 hours, then the solvent removed under reduced pressure and the residue partitioned between ethyl acetate and 1N HCl. The organic layer was separated, washed with saturated sodium hydrogen carbonate, brine, dried (MgSO₄), filtered and concentrated to yield the title compound which was used in the next step without further purification.

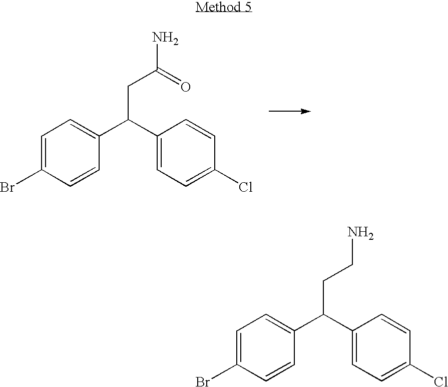

Under a nitrogen atmosphere, the crude 3-(4-bromo-phenyl)-3-(4-chloro-phenyl)-propionamide was cooled to 0° C., and lithium aluminum hydride (0.075 g, 1.97 mmol) and diethyl ether (3 ml) were added. With cooling, aluminum chloride (0.23 g, 1.69 mmol) was dissolved in diethyl ether (2 ml) and added. The reaction mixture was stirred for 16 hours, quenched with addition of water, basified (2N NaOH) and extracted with ethyl acetate. The organic layer was separated, dried (MgSO₄), filtered and concentrated, the crude product was purified over Phenomenex_Strata_SCX column chromatography eluting with methanol followed by 2N ammonia in methanol to afford the desired product.

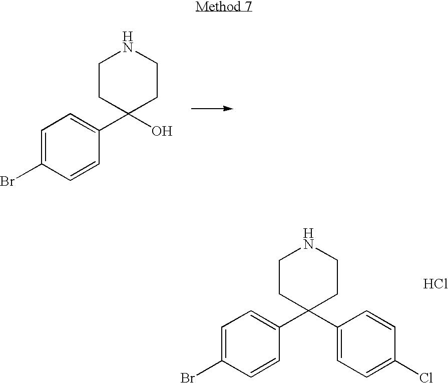

A suspension of 4-(4-bromo-phenyl)-piperidin-4-ol (4.02 g, 15.7 mmol) in chlorobenzene (30 ml) was added dropwise to a suspension of aluminium chloride (7.32 g, 54.9 mmol) in chlorobenzene (10 ml) at 0° C. The reaction mixture was stirred at 0° C. for 2 hours, quenched by addition of ice and then methyl t-butyl ether was added. After stirring for 1 hour, the precipitate was collected by filtration and washed with water, methyl t-butyl ether and water to afford the desired compound.

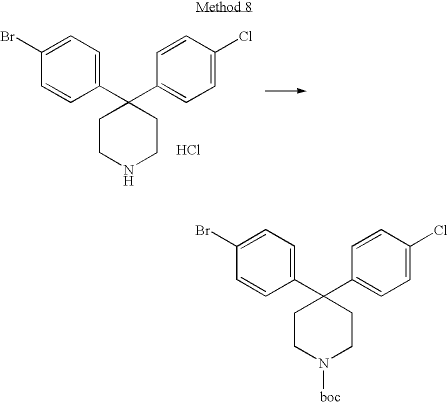

To a suspension of 4-(4-bromo-phenyl)-4-(4-chloro-phenyl)-piperidine (10 g, 25.8 mmol) in dichloromethane (150 ml) was added triethylamine (4.3 ml, 31.0 mmol) and di-tert-butyl dicarbonate (6.2 g, 28.4 mmol). After stirring for 72 hours, water was added and the organic layer was removed. The organic layer was washed with water then saturated sodium chloride solution before drying (MgSO₄) and concentrating in vacuo to furnish the desired compound as a white solid.

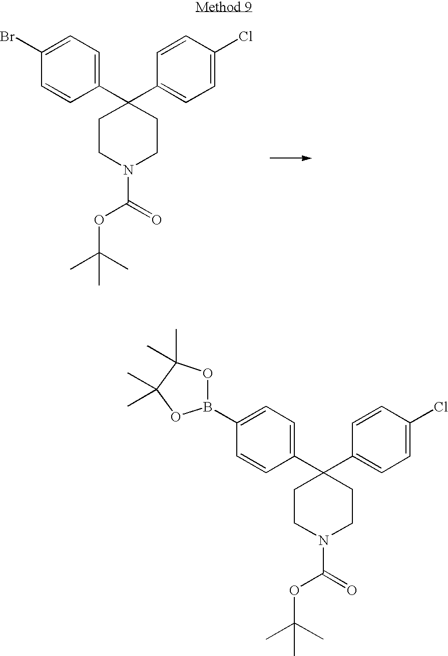

A mixture of 4-(4-bromo-phenyl)-4-(4-chloro-phenyl)-piperidine-1-carboxylic acid tert-butyl ester (5.0 g, 11.1 mmol), bis(pinacolato)diboron (2.8 g, 11.1 mmol), potassium acetate (3.3 g, 33.3 mmol) and [1,1′-bis(diphenylphosphino)-ferrocene]dichloro palladium(II) (406 mg, 0.55 mmol) was heated to 80° C. under nitrogen for 2.5 hours. The reaction was then allowed to cool, diluted with ethyl acetate then filtered under suction. The solid was triturated with ethyl acetate to furnish the desired compound as a beige solid.

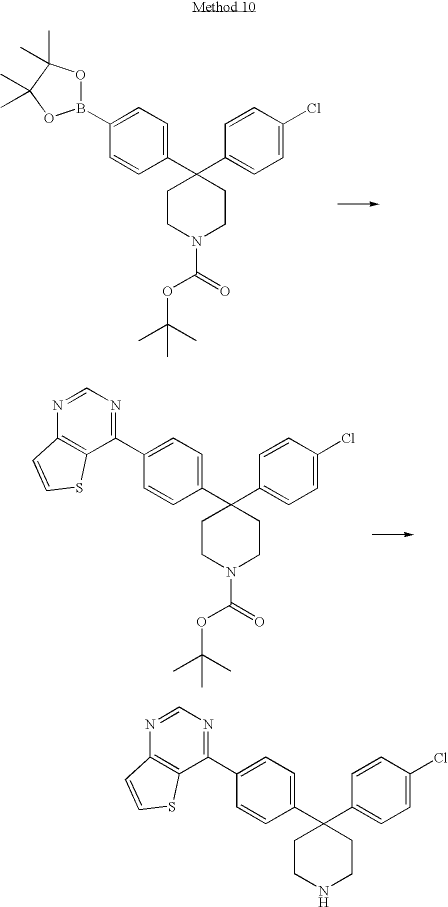

A mixture of 4-(4-chloro-phenyl)-4-[4-(4,4,5,5-tetramethyl-[1,3,2]dioxaborolan-2-yl)-phenyl]-piperidine-1-carboxylic acid tert-butyl ester (200 mg, 0.4 mmol), bis(tri-t-butylphosphine)palladium (0) (6 mg, 3 mol %), 4-chloro-thieno[3,2-d]-pyrimidine (0.5 mmol), potassium carbonate (299 mg, 1.4 mmol), ethanol (1.1 ml), toluene (1.1 ml) methanol (1.6 ml) and water (1.5 ml) is heated in a CEM Explorer™ microwave to 80° C. for 30 minutes using ≦50 watts power. The solvents are removed and the residue is partitioned between ethyl acetate and water. The aqueous layer is extracted with ethyl acetate and the combined organic layers are washed with brine, dried (MgSO₄) and concentrated under reduced pressure. The crude reaction mixture is purified by SCX ion exchange column eluting with an ammonia-dichloromethane-methanol mixture to furnish the protected amine. The protecting group is removed by stirring at room temperature in dichloromethane (1 ml) and trifluoroacetic acid (1 ml) for 30 minutes before concentrating and re-concentrating from methanol (×3). The residue is purified by silica column chromatography eluting with a gradient from DMAW90 to DMAW60.

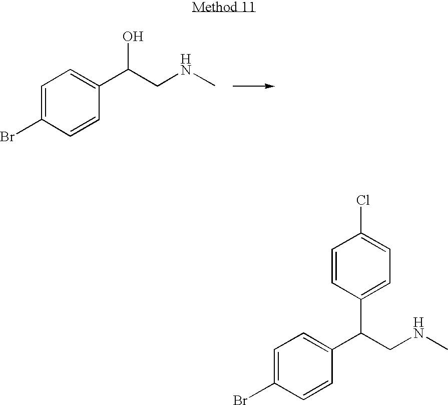

Aluminium chloride (278 mg, 2.087 mmol) was added portionwise to a stirred solution of 1-(4-bromo-phenyl)-2-methylamino-ethanol (160 mg, 0.696 mmol) in chlorobenzene (3 ml) and the reaction mixture was stirred at room temperature for 17 hours. Water (2 ml) was added dropwise and the reaction mixture was then partitioned between dichloromethane (100 ml) and saturated NaHCO₃ (30 ml). The organic layer was dried (MgSO₄), filtered and concentrated under reduced pressure. The crude product was then purified by Phenomenex Strata SCX column chromatography eluting with methanol followed by 2N ammonia in methanol to afford the desired product.

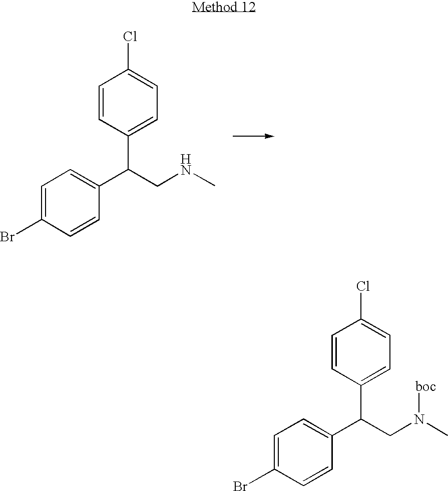

To a solution of [2-(4-bromo-phenyl)-2-(4-chloro-phenyl)-ethyl]-methyl-amine (4.3 g, 13.3 mmol) in dichloromethane at room temperature (150 ml) was added triethylamine (2.22 ml, 16 mmol) and di-tert-butyl dicarbonate (3.2 g, 15 mmol). The mixture was stirred for 3 hours whereupon water was added. The organic liquors were separated then concentrated in vacuo. The residue was purified by silica column chromatography using a gradient from 2-15% ethyl acetate/petrol furnishing the desired compound as a colourless oil.

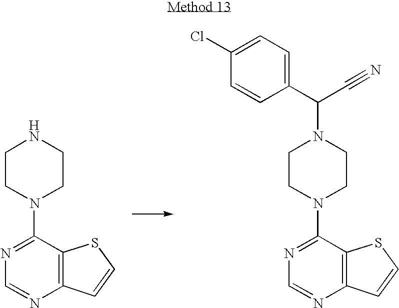

To a solution of 4-chlorobenzyl cyanide (1 g, 6.60 mmol) and sodium bromate (1.99 g, 13.19 mmol) in ethyl acetate (14 ml)/water (10 ml) was added sodium bisulphite (1.37 g, 13.19 mmol) in water (21 ml) dropwise over 15 minutes at room temperature. After stirring for 4 hours the reaction mixture was poured into diethyl ether and the phases were separated. The aqueous was extracted with diethyl ether (×2) and the organic layers were combined, washed with aqueous saturated sodium thiosulphate, dried (MgSO₄) and the solvent was removed in vacuo to yield bromo-(4-chloro-phenyl)-acetonitrile which was used in the next stage. [Ref: JOC, 1998, 63, 6023-6026]

To a solution of 4-piperazin-1-yl-thieno[3,2-d]pyrimidine (83 mg, 0.38 mmol, 0.9 equiv.) in acetonitrile (1.4 ml) was added bromo-(4-chloro-phenyl)-acetonitrile (97 mg, 0.42 mmol, 1.0 equiv), tetrabutylammonium iodide (8 mg, 0.08 mmol, 0.05 equiv.) and potassium carbonate (70 mg, 0.50 mmol, 1.2 equiv.). The mixture was stirred at room temperature overnight. The solvent was removed under reduced pressure and the resultant crude material was partitioned between ethyl acetate and water. The layers were separated and the organic layer was washed with brine, dried (MgSO₄) and concentrated under reduced pressure. The resultant crude material was purified by column chromatography eluting with 50% ethyl acetate-petrol to afford slightly impure (4-chloro-phenyl)-(4-thieno[3,2-d]pyrimidin-4-yl-piperazin-1-yl)-acetonitrile.

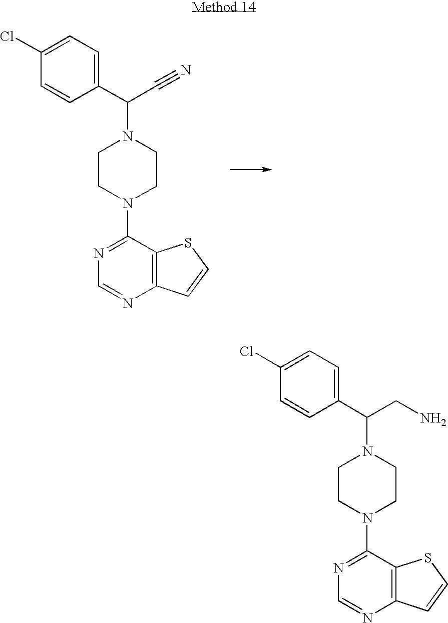

To a solution of crude (4-chloro-phenyl)-(4-thieno[3,2-d]pyrimidin-4-yl-piperazin-1-yl)-acetonitrile (from Method 22) in toluene (42 ml) was added lithium aluminum hydride (159 mg, 4.2 mmol, 10.0 equiv) portion-wise. The reaction mixture was heated to reflux for 4 hours. The reaction was quenched very slowly with an aqueous solution of 2N sodium hydroxide followed by the addition of ethyl acetate. The mixture was filtered and the layers were separated and the organic layer was washed with brine, dried (MgSO₄) and concentrated under reduced pressure. The resultant crude material was purified by column chromatography using (gradient elution) 100% DCM to DMAW90 followed by further preparative HPLC to afford the desired product.

### Examples 1 to 4

By following the methods described above, the compounds of Examples 1 to 4 were prepared.

| | | | | NMR | |
|---|---|---|---|---|---|
| Example | Chemical | | Method of | characterisation | |
| No. | structure | Chemical name | preparation | data | MS |
| 1 | 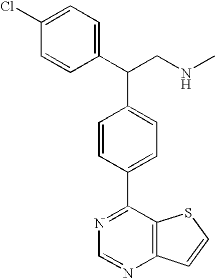 | [2-(4-Chloro- phenyl)-2-(4- thieno[3,2-d]- pyrimidin-4-yl- phenyl)-ethyl]- methyl-amine bis- trifluoroacetate | Methods 11, 12 and 10 - using 4- chloro-thieno[3,2- d]-pyrimidine | ¹H NMR (Me-d₃- OD) 9.22 (1 H, s), 8.40 (1 H, d), 8.24 (2 H, d), 7.68 (3 H, m), 7.43 (4 H, s), 4.51 (1 h, t), 3.88 (2 H, m), 2.80 (3 H, s) | MS: [M + H]⁺ 308 |
| 2 | 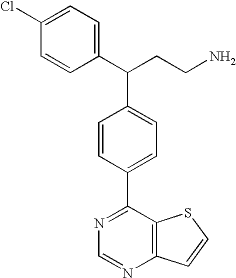 | 3-(4-Chloro- phenyl)-3-(4- thieno[3,2- d]pyrimidin-4- yl-phenyl)- propylamine | Methods 1 to 5 and 8 to 10 - using 4- chlorothieno[3,2- d]pyrimidine | ¹H NMR (Me-d₃- OD) 9.20 (1 H, s), 8.40 (1 H, d), 8.19 (2 H d), 7.65 (1 H, d), 7.60 (2 H, d), 7.35 (4 H, m), 4.22 (1 H, t), 2.71 (2 H, m), 2.38 (2 H, m) | MS: [M + H]⁺ 380 |
| 3 | 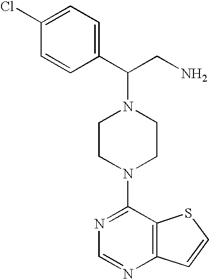 | 2-(4-Chloro- phenyl)-2-(4- thieno[3,2- d]pyrimidin-4- yl-piperazin-1- yl)-ethylamine formate | Methods 13 and 14 | ¹H NMR (Me-d₃- OD) 8.50 (1 H, br s), 8.41 (1 H, s), 8.03 (1 H, d), 7.44 (2 H, d), 7.36 (1 H, d), 7.38 (2 H, d), 4.13-4.06 (4 H, m), 3.99 (1 H, dd), 3.72 (1 H, t), 3.16 (1 H, dd), 2.77- 2.72 (2 H, m), 2.48-2.41 (2 H, m) | MS: [M + H]⁺ 374 |
| 4 | 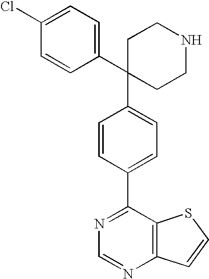 | 4-{4-[4-(4- Chloro-phenyl)- piperidin-4-yl]- phenyl}- thieno[3,2- d]pyrimidine formate | Methods 7 to 10 - using 4-chloro- thieno[3,2-d]- pyrimidine | ¹H NMR (Me-d₃- OD) 9.20 (1 H, s), 8.52 (1 H, s), 8.40 (1 H, d), 8.20 (2 H, d), 7.68 (3 H, m), 7.43 (2 H, d), 7.40 (2 H, d), 3.26 (4 H, m), 2.89 (4 H, m) | MS: [M + H]⁺ 406 |

### Example 5

### 5A. 4-tert-Butoxycarbonylamino-4-(4-chloro-benzylcarbamoyl)-piperidine-1-carboxylic Acid Tert-Butyl Ester

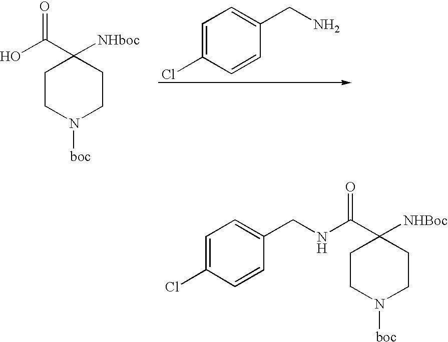

Dry DMF (1 mL) was added to a mixture of 4-tert-butoxycarbonylamino-piperidine-1,4-dicarboxylic acid mono tert-butyl ester (151 mg, 0.44 mmol) and HATU (220 mg, 0.58 mmol) under nitrogen. N-Ethyldiisopropylamine (0.38 mL, 2.1 mmol) was added to the solution and the reaction mixture was stirred for 15 min. 4-Chlorobenzylamine (70 uL, 0.57 mmol) was added and the solution was stirred for 23 h at rt and under nitrogen. The reaction mixture was partioned between dichloromethane (10 mL) and water (10 mL). The aqueous phase was further extracted with dichloromethane (20 mL). The combined organic layers were dried (Mg₂SO₄), filtered and concentrated. Flash column chromatography on silica, eluting with 4% methanol in dichloromethane, gave 4-tert-butoxycarbonylamino-4-(4-chloro-benzylcarbamoyl)-piperidine-1-carboxylic acid tert-butyl ester (177 mg, 0.38 mmol, 86%). LC-MS (LCT2) m/z 490 [M+Na⁺], Rₜ 8.09 min.

### 5B. 4-Amino-piperidine-4-carboxylic acid 4-chloro-benzylamide Dihydrochloride

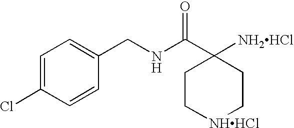

A 4M solution of HCl in dioxane (7.7 ml, 31 mmol) was added dropwise to a solution of 4-tert-butoxycarbonylamino-4-(4-chloro-benzylcarbamoyl)-piperidine-1-carboxylic acid tert-butyl ester (96 mg, 0.20 mmol) in methanol (7.7 mL) and stirred at rt for 17 h. The solvents were concentrated to give 4-amino-piperidine-4-carboxylic acid 4-chloro-benzylamide dihydrochloride (71 mg, 0.20 mmol, 100%) that was used in the next step without further purification. ¹H NMR (500 MHz, CD₃OD): 2.18 (2H, m), 2.64 (2H, m), 3.44 (4H, m), 4.47 (2H, s), 7.36 (4H, m).

### 5C. 4-Amino-1-thieno[3,2-d]pyrimidin-4-yl-piperidine-4-carboxylic acid 4-chloro-benzylamide

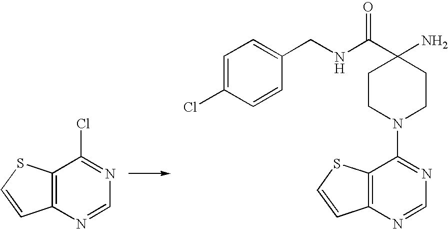

A solution of 4-chloro-thieno[3,2-d]pyrimidine (0.016 g, 0.094 mmol), 4-amino-piperidine-4-carboxylic acid 4-chloro-benzylamide (0.030 g, 0.098 mmol) and triethylamine (0.07 mL, 0.50 mmol) in n-butanol (0.5 mL) was heated by microwave irradiation to 120° C. for 1 h. The cooled solution was filtered through SCX-II acidic resin, eluting with methanol then with 1M ammonia-methanol. The basic fractions were combined. Preparative TLC, eluting with 5% methanol-dichloromethane, gave 4-amino-1-thieno[3,2-d]pyrimidin-4-yl-piperidine-4-carboxylic acid 4-chloro-benzylamide as an off-white solid (0.014 g, 33%). LC/MS: (LCT1) [M+H]⁺ 402, Rₜ 2.98 min. ¹H (500 MHz, MeOD) δ 7.89 (1H, s), 7.45 (1H, d, J=6 Hz), 6.82 (1H, d, J=6 Hz), 6.75-6.65 (4H, m), 4.10-3.95 (2H, m), 3.81 (2H, s), 3.20-3.15 (2H, m), 1.70-1.60 (2H, m), 1.15-1.05 (2H, m)

### Example 6

### 1-Thieno[3,2-d]pyrimidin-4-ylpiperidin-4-ylamine

### 6A. (1-Thieno[3,2-d]pyrimidin-4-yl-piperidin-4-yl)carbamic Acid Tert-Butyl Ester

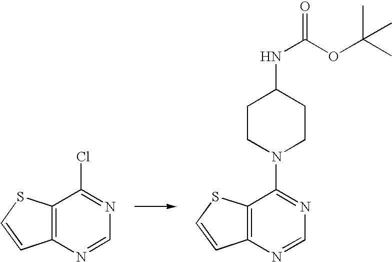

The title compound was prepared using the method of Example 5 (AT11980) except that piperidin-4-yl-carbamic acid tert-butyl ester was used in place of 4-amino-piperidine-4-carboxylic acid 4-chloro-benzylamide. LC/MS: (LCT1) [M+H]⁺ 334, Rₜ 4.62 min

### 6B. 1-Thieno[3,2-d]pyrimidin-4-ylpiperidin-4-ylamine

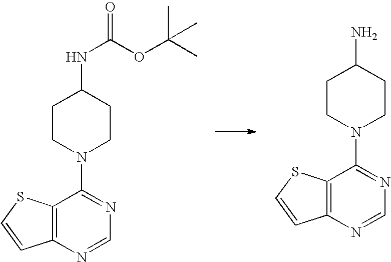

A solution of the product of Example 6A in 2M HCl (2 ml) was stirred at room temperature for 2 hours, and then evaporated to dryness. Solid phase extraction on SCX-II acidic resin, eluting with MeOH and then 1M NH₃ in MeOH, gave the deprotected product. LC/MS (LCT1): [M+H]⁺ 234, Rₜ 0.85 min. ¹H (250 MHz, MeOD) δ 8.45 (1H, s), 8.04 (1H, d, J=5.5 Hz), 7.38 (1H, d, J=5.5 Hz), 4.95-4.81 (2H, m), 3.34-3.29 (2H, m), 3.08-2.98 (1H, m), 2.06-2.00 (2H, m), 1.52-1.36 (2H, m).

### Example 7

### 4-(4-Chlorobenzyl)-1-thieno[3,2-d]pyrimidin-4-ylpiperidin-4-ylamine

### 7A. 4-(4-Chlorobenzyl)piperidine-1,4-dicarboxylic Acid 1-tert-butyl Ester 4-methyl Ester

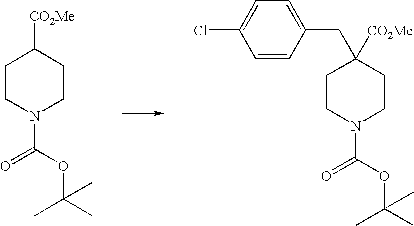

To a solution of isopropylamine (3.71 ml, 26.45 mmol) in THF (110 ml) at 0° C. was added n-butyllithium (10.1 ml of a 2.5M solution in hexanes, 25.25 mmol). The resulting LDA solution was added via cannula to a solution of piperidine-1,4-dicarboxylic acid 1-tert-butyl ester 4-methyl ester (5.85 g, 24.04 mmol) in THF (110 ml) and HMPA (20 ml) at −78° C. and stirring was continued for 1 hour. 4-Chlorobenzyl chloride (6.4 ml, 50.49 mmol) in THF (20 ml) was added and the solution was warmed to room temperature over 2 hours. After stirring for 18 hours, saturated aqueous ammonium chloride (500 ml) was added and the aqueous phase was extracted with diethyl ether (2×200 ml). The organic phases were combined, dried over magnesium sulphate and concentrated to dryness. Purification by silica column chromatography (0.5% methanol in DCM) gave the ester as an oil (3.03 g, 34%). LC-MS (LCT1) m/z 390 [M+Na⁺], Rₜ 8.02 min.

### 7B. 4-(4-Chlorobenzyl)piperidine-1,4-dicarboxylic Acid Mono-Tert-Butyl Ester

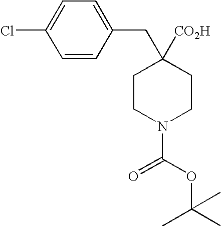

To a solution of 4-(4-chlorobenzyl)piperidine-1,4-dicarboxylic acid 1-tert-butyl ester 4-methyl ester (1.515 g, 4.117 mmol) in dioxane (20 ml), methanol (10 ml) and water (10 ml) at room temperature was added lithium hydroxide monohydrate (3.455 g, 82.341 mmol). After stirring at 50° C. for 2 days the solution was acidified to pH 6 with 2M HCl and the resulting white precipitate was extracted with diethyl ether (2×100 ml). The organic phases were combined, dried over sodium sulphate and concentrated to dryness, to give the acid as a white solid (1.460 g, 100%). LC-MS (LCT) m/z 376 [M+Na⁺], Rₜ 7.62 min.

### 7C. 4-(4-Chlorobenzyl)piperidin-4-yl Amine

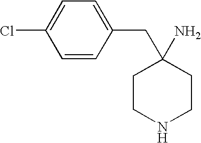

To a mixture of the acid (1.46 g, 4.126 mmol) and triethylamine (1.15 ml, 8.252 mmol) in THF (41 ml) at −15° C. was added isobutyl chloroformate (0.812 ml, 6.189 mmol). After 1 hour, a solution of sodium azide (0.536 g, 8.252 mmol) in water (10 ml) was added and the solution was warmed to room temperature overnight. Water (100 ml) was added and the aqueous phase was extracted with diethyl ether (3×50 ml). The organic phases were combined, washed with saturated sodium bicarbonate (50 ml) and dried over sodium sulphate. Toluene (100 ml) was added and the overall volume was reduced to approximately 90 ml. The resulting solution was warmed to 90° C. for 2 h, then cooled and added to 10% hydrochloric acid (70 ml). The biphasic mixture was warmed to 90° C. for 24 hours. The organic phase was separated and concentrated to dryness to give the crude amine salt (1.109 g).

The crude amine salt was dissolved in 2M NaOH (20 ml) and di-tert-butyl dicarbonate (1.61 g, 7.391 mmol) added. After 2 days the aqueous phase was extracted with diethyl ether (2×50 ml). The organic phases were combined, washed with 1M HCl (20 ml), saturated sodium bicarbonate (20 ml) and brine (20 ml), then dried over magnesium sulphate and concentrated. Purification by column chromatography (50% diethyl ether in hexanes) gave the doubly BOC-protected amine (0.685 g), which was subsequently deprotected by stirring with 4M HCl in dioxane (10 ml) and methanol (10 ml) at room temperature for 2 days. Concentration gave the desired amine as the bis-hydrochloride salt (0.492 g, 40% from acid). ¹H NMR (MeOD) δ 7.48-7.44 (m, 2H), 7.35-7.32 (m, 2H), 3.53-3.47 (4H, m), 3.21 (s, 2H), 2.18-2.13 (4H, m).

### 7D. 4-(4-Chlorobenzyl)-1-thieno[3,2-d]pyrimidin-4-ylpiperidin-4-ylamine

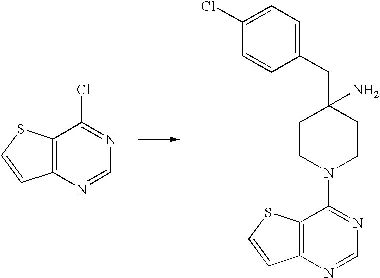

The product of Example 7D was reacted with 4-chloro-thieno[3,2-d]pyrimidine according to the method of Example 5C to give the title compound. LC/MS: (LCT2) [M+H]⁺ 359, Rₜ 2.73 min. ¹H (500 MHz, MeOD) δ 7.51 (1H, d, J=2.0 Hz), 7.10 (1H, dd, J=5.5, 2.0 Hz), 6.46-6.31 (5H, m), 3.51-3.48 (2H, m), 2.94-2.89 (2H, m), 1.86 (2H, s), 0.87-0.82 (2H, m), 0.66-0.64 (2H, m).

### Example 8

### C-(4-Chlorophenyl)-C-(1-thieno[3,2-d]pyrimidin-4-ylpiperidin-4-yl)methylamine

### 8A. 4-(4-Chlorobenzoyl)piperidine-1-carboxylic Acid Tert-Butyl Ester

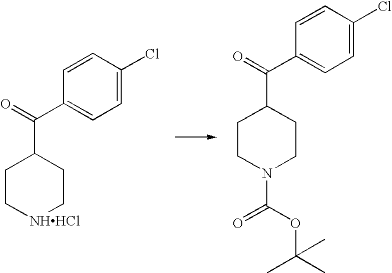

To a mixture of (4-chlorophenyl)piperidin-4-ylmethanone hydrochloride (0.996 g, 3.828 mmol) and triethylamine (2.7 ml, 19.142 mmol) in acetonitrile (15 ml) at room temperature was added di-tert-butyl dicarbonate (1.003 g, 4.594 mmol). After 16 hours at room temperature, the mixture was evaporated to dryness and then partitioned between ethyl acetate (50 ml) and 1M hydrochloric acid (20 ml). The organic phase was separated and washed successively with saturated aqueous sodium bicarbonate (20 ml), then brine (20 ml), before being dried over magnesium sulfate and concentrated to dryness. The crude material was purified by silica column chromatography (60% diethyl ether in hexanes) to give the ketone as an oil (1.116 g, 90%). LC/MS: (LCT1) Rₜ 7.42 [M+H]⁺ 323.

### 8B. 4-[Amino-(4-chlorophenyl)methyl]piperidine-1-carboxylic Acid Tert-Butyl Ester

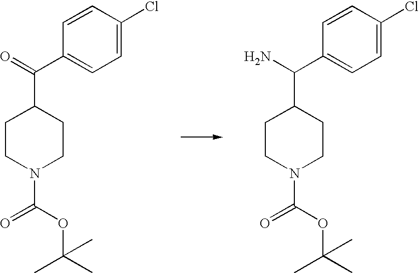

To a mixture of 4-(4-chlorobenzoyl)piperidine-1-carboxylic acid tert-butyl ester (1.116 g, 3.446 mmol) and ammonium acetate (3.188 g, 41.358 mmol) in methanol (34 ml) at room temperature was added sodium cyanoborohydride (0.866 g, 13.786 mmol). After refluxing for 20 hours, the mixture was cooled, concentrated and stirred with 1M sodium hydroxide (100 ml). The aqueous phase was extracted with diethyl ether (3×75 ml), with the organic layers being combined, dried over sodium sulfate and concentrated to dryness. The crude material was purified by silica column chromatography (15% methanol in DCM) to give the amine as an oil (0.913 g, 82%). LC/MS (LCT1): Rₜ 5.56 [M-Boc-NH₂]⁺208.

### 8C. C-(4-Chlorophenyl)-C-piperidin-4-ylmethylamine Hydrochloride

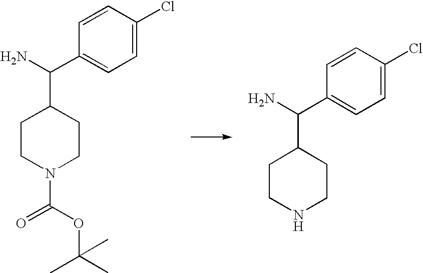

To a solution of 4-[amino-(4-chlorophenyl)methyl]piperidine-1-carboxylic acid tert-butyl ester (0.192 g, 0.591 mmol) in methanol (6 ml) at room temperature was added 2M hydrochloric acid (6 ml). After stirring for 16 hours the solution was evaporated to dryness to give the amine salt as a white foam (0.174 g, 99%). ¹H NMR (MeOD) δ 1.40-1.82 (2H, m), 2.22-2.50 (2H, m), 2.90-3.17 (2H, m), 3.35-3.61 (2H, m), 4.22 (1H, d, 9.5 Hz), 7.53-7.61 (4H, m).

### 8D. C-(4-Chlorophenyl)-C-(1-thieno[3,2-d]pyrimidin-4-ylpiperidin-4-yl)methylamine

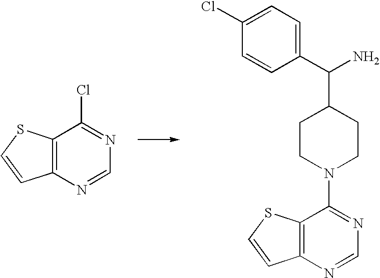

The product of Example 8C was reacted with 4-chloro-thieno[3,2-d]pyrimidine according to the method of Example 5C to give the title compound. LC/MS: (LCT2) [M+H]⁺ 358, Rₜ 3.51 min. ¹H (250 MHz, MeOD) δ 8.42 (1H, s), 8.01 (1H, d, J=5.5 Hz), 7.38-7.31 (5H, m), 5.00-4.79 (2H, m), 3.60 (1H, d, J=8.5 Hz), 3.25-3.02 (2H, m), 2.18-1.89 (2H, m), 1.48-1.17 (3H, m).

### Example 9

### 1-Thieno[2,3-d]pyrimidin-4-ylpiperidin-4-ylamine

### 9A. (1-Thieno[2,3-d]pyrimidin-4-yl-piperidin-4-yl)carbamic Acid Tert-Butyl Ester

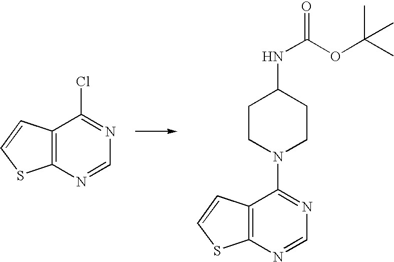

To a mixture of 4-chlorothieno[2,3-d]pyrimidine (0.55 g, 0.322 mmol) and piperidin-4-yl carbamic acid tert-butyl ester (0.129 g, 0.646 mmol) in n-butanol (3.2 ml) was added triethylamine (0.225 mL, 1.617 mmol). After heating at 100° C. for 48 h, solvent was removed and the resulting solid was purified by silica column chromatography (5% methanol-dichloromethane eluant) to give the desired product as an off-white solid (0.108 g, 100%). LC/MS: (LCT1) [M+H]⁺ 334, Rₜ 6.54 min

### 9B. 1-Thieno[2,3-d]pyrimidin-4-ylpiperidin-4-ylamine

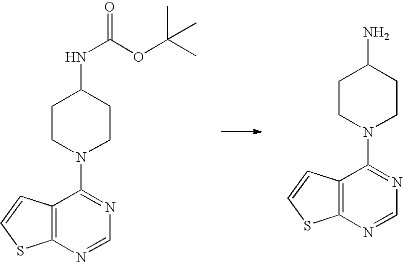

A solution of (1-thieno[2,3-d]pyrimidin-4-yl-piperidin-4-yl) carbamic acid tert-butyl ester (0.108 g, 0.322 mmol) in 1M HCl in diethyl ether (3 mL) and methanol (3 mL) was stirred at rt for 24 h, then evaporated to dryness. Solid phase extraction on SCX-II acidic resin, eluting with MeOH then 1M ammonia in methanol, gave the deprotected amine as a white solid (0.076 g, 100%). LC/MS (LCT1): [M+H]⁺ 234, Rₜ 2.24 min. ¹H (250 MHz, MeOD) δ 8.36 (1H, s), 7.54 (1H, d, J=6.0 Hz), 7.49 (1H, d, J=6.0 Hz), 4.70-4.64 (2H, m), 3.35-3.26 (2H, m), 3.07-2.95 (1H, m), 2.04-1.99 (2H, m), 1.54-1.38 (2H, m).

### Example 10

### 4-(4-Amino-piperidin-1-yl)-1H-pyrrolo[2,3-b]pyridine-2,3-dione and 4-(4-amino-piperidin-1-yl)-1,3-dihydro-pyrrolo[2,3-b]pyridin-2-one

### 10A. 1H-Pyrrolo[2,3-b]pyridine 7-oxide

A solution of 1H-pyrrolo[2,3-b]pyridine (6.35 g, 53 mmol) in ethyl acetate (200 mL) was cooled to 0-5° C. in an ice bath. To the cooled solution was added mCPBA (14 g, 64 mmol) over 10 min. The resulting solution was warmed to room temperature until the starting material was totally consumed (2.5 h). The resulting slurry was filtered to collect the N-oxide as the meta-chlorobenzoic acid salt. The solid was washed with additional ethyl acetate and dried to provide 10.4 g (36 mmol). A suspension of the 7-hydroxy-1H-pyrrolo[2,3-b]pyridinium m-chlorobenzoate (10.4 g, 36 mmol) in water (100 mL) was basified to pH 11 with saturated aqueous K₂CO₃. The mixture was cooled (+4° C.) overnight to give crystals which were collected and washed with hexane followed by diethyl ether to yield 1H-pyrrolo[2,3-b]pyridine 7-oxide (3.22 g, 24 mmol, 67%). LC-MS (LCT1) m/z 135.1 [M+H⁺], Rₜ 2.62 min.

### 10B. 4-Chloro-1H-pyrrolo[2,3-b]pyridine

Methanesulphonyl chloride (5 mL, 64 mmol) was added dropwise to a solution of 1H-pyrrolo[2,3-b]pyridine 7-oxide (3.18 g, 24 mmol) in DMF (16 mL) and heated to 50° C. The resulting mixture was heated at 72° C. overnight. The reaction mixture was cooled to 30° C. and quenched with water (50 mL). The mixture was cooled in an ice bath and sufficient 10M aqueous NaOH was added to raise the pH to 7. The resulting slurry was warmed to room temperature, stirred for 15 min, and then filtered to collect the product. The solid was washed with water and dried in vacuo to give 4-chloro-1H-pyrrolo[2,3-b]pyridine (2.97 g, 19.5 mmol, 81%). LC-MS (LCT1) m/z 153.03 [M+H⁺], Rₜ 5.77 min.

### 10C. 3,3-Dibromo-4-chloro-1,3-dihydro-pyrrolo[2,3-b]pyridin-2-one

To a stirred solution of 4-chloro-1H-pyrrolo[2,3-b]pyridine (1 g, 6.5 mmol) in 50 mL of t-butanol was added in small portions pyridinium tribromide 90% (7.24 g, 22.6 mmol) over 7 min. The reaction was stirred at room temperature overnight. t-Butanol was removed in vacuo and the resulting residue was dissolved in ethyl acetate-water (200 mL:200 mL). The organic layer was separated and the aqueous layer was further extracted with ethyl acetate (2×100 mL). The combined organic extracts were washed with water, brine, dried (MgSO₄) and concentrated in vacuo to give 3,3-dibromo-4-chloro-1,3-dihydro-pyrrolo[2,3-b]pyridin-2-one (2.17 g, 6.6 mmol, 100%). LC-MS (LCT1) m/z 326.78 [M+H⁺], Rₜ 5.75 mm.

### 10D. 4-Chloro-1,3-dihydro-pyrrolo[2,3-b]pyridin-2-one

A suspension of 3,3-dibromo-4-chloro-1,3-dihydro-pyrrolo[2,3-b]pyridin-2-one (1.05 g, 3.15 mmol), ethanol (120 mL) and 10% Pd/C (391 mg) was hydrogenated at room temperature and room pressure for 6 h 15 min. The reaction mixture was filtered through a pad of celite and washed with methanol. The solvents were evaporated and the crude material was partitioned between dichloromethane (50 mL) and saturated aqueous sodium bicarbonate (50 mL). After separating the two phases the aqueous layer was further extracted with dichloromethane (2×50 mL). The combined organic layers were dried (MgSO₄), filtered and evaporated to give 4-chloro-1,3-dihydro-pyrrolo[2,3-b]pyridin-2-one (328 mg, 1.94 mmol, 62%). LC-MS (LCT1:15 min run) m/z 169.02 [M+H⁺], Rₜ 3.96 min.

### 10E. [1-(2,3-Dioxo-2,3-dihydro-1H-pyrrolo[2,3-b]pyridin-4-y)-piperidin-4-yl]-carbamic Acid Tert-Butyl Ester

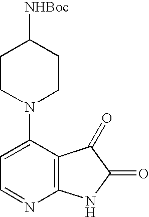

### Method A

A degassed mixture of piperidin-4-yl-carbamic acid tert-butyl ester (76 mg, 0.6 mmol), 4-chloro-1,3-dihydro-pyrrolo[2,3-b]pyridin-2-one (50 mg, 0.3 mmol), triethylamine (0.31 mL, 2.1 mmol) and n-butanol (3 mL) was stirred at 100° C. for 3.5 h. Solvents were evaporated and the crude mixture was purified by flash silica column chromatography, eluting with 5% methanol-dichloromethane, to give [1-(2,3-dioxo-2,3-dihydro-1H-pyrrolo[2,3-b]pyridin-4-yl)-piperidin-4-yl]-carbamic acid tert-butyl ester (24.4 mg, 0.07 mmol, 24%). LC-MS (LCT2) m/z 347.22 [M+H⁺], Rₜ 5.80 min.

### Method B

A mixture of 4-chloro-1,3-dihydro-pyrrolo[2,3-b]pyridin-2-one (25 mg, 0.15 mmol), piperidin-4-yl-carbamic acid tert-butyl ester (38 mg, 0.3 mmol) and N-methyl-pyrrolidinone (0.2 ml) was irradiated in the microwave for 1 h at 155° C. The solution was diluted in methanol and purified on SCX-II acidic resin eluting with methanol and then with 2M ammonia-methanol. The crude material was further purified by flash silica column chromatography, eluting with 10% methanol-dichloromethane, to afford a mixture of [1-(2,3-dioxo-2,3-dihydro-1H-pyrrolo[2,3-b]pyridin-4-yl)-piperidin-4-yl]-carbamic acid tert-butyl ester and [1-(2-oxo-2,3-dihydro-1H-pyrrolo[2,3-b]pyridin-4-yl)-piperidin-4-yl]-carbamic acid tert-butyl ester. These compounds were separated by preparative HPLC (Discovery C18 Supelco HPLC column 15 cm×10 mm, 5 μL; acetonitrile/water gradient solvent system).

[1-(2,3-dioxo-2,3-dihydro-1H-pyrrolo[2,3-b]pyridin-4-yl)-piperidin-4-yl]-carbamic acid tert-butyl ester: 6.4 mg, 0.018 mmol, 12%, LC-MS (LCT2) m/z 347.22 [M+H⁺], Rₜ 5.90 min

[1-(2-oxo-2,3-dihydro-1H-pyrrolo[2,3-b]pyridin-4-yl)-piperidin-4-yl]-carbamic acid tert-butyl ester: 1.6 mg, 0.005 mmol, 3%, LC-MS (LCT2) m/z 333.27 [M+H+], Rₜ 3.43 min

### 10F. 4-(4-Amino-piperidin-1-yl)-1H-pyrrolo[2,3-b]pyridine-2,3-dione

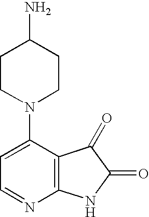

Trifluoroacetic acid (0.5 ml, 6.7 mmol) was added dropwise to a solution of [1-(2,3-dioxo-2,3-dihydro-1H-pyrrolo[2,3-b]pyridin-4-yl)-piperidin-4-yl]-carbamic acid tert-butyl ester (4.7 mg, 0.014 mmol) in dichloromethane (1 mL). The solution was stirred at rt for 45 min. The solvents were concentrated and the crude mixture was purified on SCX-II acidic resin, eluting with methanol then 2M ammonia-methanol, to give 4-(4-amino-piperidin-1-yl)-1H-pyrrolo[2,3-b]pyridine-2,3-dione (3.5 mg, 0.014 mmol, 100%). LC-MS (LCT2) m/z 247.19 [M+H⁺], Rₜ 0.79 min. ¹H (500 MHz, MeOD) δ 7.84 (d, J=5 Hz, 1H), 6.60 (d, J=5 Hz, 1H), 4.22-4.28 (m, 2H), 3.14-3.21 (m, 2H), 2.96-3.08 (m, 1H), 2.00-2.05 (m, 2H), 1.30-1.62 (m, 2H).

### Example 11

### 8-[4-Amino-4-(4-chloro-benzyl)-piperidin-1-yl]-4H-pyrido[3,2-b][1,4]oxazin-3-one

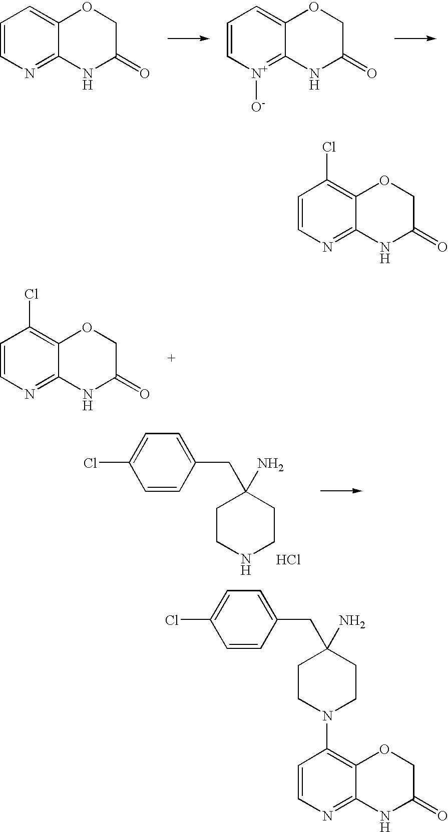

### 11A. 5-Oxy-4H-pyrido[3,2-b][1,4]oxazin-3-one

m-Chloroperbenzoic acid (2.76 g, 16.0 mmol) was added in portions to a stirred suspension of 4H-pyrido[3,2-b][1,4]oxazin-3-one (2 g, 13.3 mmol) in a mixture of ethyl acetate (150 mL) and acetone (100 mL) at room temperature. After stirring at room temperature for 2 days, a fine precipitate was formed. The solid was collected, washed with acetone and dried in vacuo, to yield 5-oxy-4H-pyrido[3,2-b][1,4]oxazin-3-one (1.9 g, 86%) as a light grey powder. LC-MS (LCT2) m/z 167.1[M+H⁺], Rₜ 1.69 min. ¹H (500 MHz, d₆-DMSO) δ 8.00 (1H, dd, J=6.3, 1.4 Hz), 7.10-6.95 (2, m), 4.72 (2H, s

### 11B. 8-Chloro-4H-pyrido[3,2-b][1,4]oxazin-3-one

Methylsulphonyl chloride (1.2 mL) was added dropwise to a solution of 5-oxy-4H-pyrido[3,2-b][1,4]oxazin-3-one (0.99 g, 6.0 mmol) in DMF (6 mL) at 50° C. The solution was heated to 80° C. and stirred for 16 h. The resulting dark solution was diluted with brine and extracted with ethyl acetate (2×50 mL). The combined organic layers were washed with aqueous NaHCO₃ (2×40 mL), brine (2×40 mL) and dried (Na₂SO₄). Solvents were evaporated to give a crude oil (0.36 g). Silica column chromatography yielded 8-chloro-4H-pyrido[3,2-b][1,4]oxazin-3-one (36 mg, 3%) as a yellow powder. LC-MS (LCT2) m/z 185.0 [M+H⁺], Rₜ 4.68 min. ¹H (500 MHz, d₆-DMSO) δ 11.40 (1H, s), 7.82 (1H, d, J=6.5 Hz), 7.12 (1H, d, J=6.5 Hz), 4.75 (2H, s).

### 11C. 8-[4-Amino-4-(4-chloro-benzyl)-piperidin-1-yl]-4H-pyrido[3,2-b][1,4]oxazin-3-one

A mixture of 8-chloro-4H-pyrido[3,2-b][1,4]oxazin-3-one (6 mg, 0.03 mmol), 4-(4-chloro-benzyl)-piperidin-4-ylamine hydrochloride (Example 7C) (9.5 mg, 0.03 mmol), triethylamine (0.024 mL) in n-butanol (1 mL) was irradiated in a CEM microwave for 2 h at 200° C. (300 W). After cooling, the solvent was evaporated. The crude product was purified on SCX-II acidic resin, eluting with ammonia-methanol, then by silica column chromatography (ethyl acetate:methanol 3:1), to yield 8-[4-amino-4-(4-chloro-benzyl)-piperidin-1-yl]-4H-pyrido[3,2-b][1,4]oxazin-3-one (1 mg, 8%). LC-MS (LCT2) m/z 373.3 [M+H⁺], Rₜ 3.60 min. ¹H (500 MHz, d₄-MeOD) δ 7.70 (1H, d, J=6.5 Hz), 7.30 (2H, d, J=7.0 Hz), 7.23 (2H, d, J=7.0 Hz), 6.64 (1H, d, J=6.5 Hz), 4.56 (2H, s), 3.48 (2H, m), 3.30 (2H, m), 2.78 (2H, s), 2.05 (2H, m), 1.60 (2H, m).

### Biological Activity

### Example 12

### Measurement of PKA Kinase Inhibitory Activity (IC₅₀)

Compounds of the invention can be tested for PK inhibitory activity using the PKA catalytic domain from Upstate Biotechnology (#14-440) and the 9 residue PKA specific peptide (GRTGRRNSI), also from Upstate Biotechnology (#12-257), as the substrate. A final concentration of 1 nM enzyme is used in a buffer that includes 20 mM MOPS pH 7.2, 40 μM ATP/γ³³P-ATP and 5 μM substrate. Compounds are added in dimethylsulphoxide (DMSO) solution to a final DMSO concentration of 2.5%. The reaction is allowed to proceed for 20 minutes before addition of excess orthophosphoric acid to quench activity. Unincorporated γ³³P-ATP is then separated from phosphorylated proteins on a Millipore MAPH filter plate. The plates are washed, scintillant is added and the plates are then subjected to counting on a Packard Topcount.

The % inhibition of the PKA activity is calculated and plotted in order to determine the concentration of test compound required to inhibit 50% of the PKB activity (IC₅₀).

The compounds of Examples 1 and 4 have IC₅₀ values of less than 1 μM.

### Example 13

### Measurement of PKB Kinase Inhibitory Activity (IC₅₀)

The inhibition of protein kinase B (PKB) activity by compounds can be determined determined essentially as described by Andjelkovic et al. (Mol. Cell. Biol. 19, 5061-5072 (1999)) but using a fusion protein described as PKB-PIF and described in full by Yang et al (Nature Structural Biology 9, 940-944 (2002)). The protein is purified and activated with PDK1 as described by Yang et al. The peptide AKTide-2T (H-A-R-K-R-E-R-T-Y-S-F-G-H-H-A-OH) obtained from Calbiochem (#123900) is used as a substrate. A final concentration of 0.6 nM enzyme is used in a buffer that includes 20 mM MOPS pH 7.2, 30 μM ATP/γ³³P-ATP and 25 μM substrate. Compounds are added in DMSO solution to a final DMSO concentration of 2.5%. The reaction is allowed to proceed for 20 minutes before addition of excess orthophosphoric acid to quench activity. The reaction mixture is transferred to a phosphocellulose filter plate where the peptide binds and the unused ATP is washed away. After washing, scintillant is added and the incorporated activity measured by scintillation counting.

The % inhibition of the PKB activity is calculated and plotted in order to determine the concentration of test compound required to inhibit 50% of the PKB activity (IC₅₀). Following the protocol described above, the IC₅₀ values of the compounds of Examples 1 to 5, 7 and 8 have been found to be less than 1 μM whilst the compounds of Examples 6 and 11 each have IC₅₀ values of less than 5 μM, and the compounds of Examples 9 and 10 have IC₅₀ values of less than 50 μM.

### Example 14

### Anti-Proliferative Activity

The anti-proliferative activities of compounds of the invention are determined by measuring the ability of the compounds to inhibition of cell growth in a number of cell lines. Inhibition of cell growth is measured using the Alamar Blue assay (Nociari, M. M, Shalev, A., Benias, P., Russo, C. Journal of Immunological Methods 1998, 213, 157-167). The method is based on the ability of viable cells to reduce resazurin to its fluorescent product resorufin. For each proliferation assay cells are plated onto 96 well plates and allowed to recover for 16 hours prior to the addition of inhibitor compounds for a further 72 hours. At the end of the incubation period 10% (v/v) Alamar Blue is added and incubated for a further 6 hours prior to determination of fluorescent product at 535 nM ex/590 nM em. In the case of the non-proliferating cell assay cells are maintained at confluence for 96 hour prior to the addition of inhibitor compounds for a further 72 hours. The number of viable cells is determined by Alamar Blue assay as before. All cell lines are obtained from ECACC (European Collection of cell Cultures) or ATCC.

### Example 15

### (i) Tablet Formulation

A tablet composition containing a compound of the formula (I) is prepared by mixing 50 mg of the compound with 197 mg of lactose (BP) as diluent, and 3 mg magnesium stearate as a lubricant and compressing to form a tablet in known manner.

### (ii) Capsule Formulation

A capsule formulation is prepared by mixing 100 mg of a compound of the formula (I) with 100 mg lactose and filling the resulting mixture into standard opaque hard gelatin capsules.

### (iii) Injectable Formulation I

A parenteral composition for administration by injection can be prepared by dissolving a compound of the formula (I) (e.g. in a salt form) in water containing 10% propylene glycol to give a concentration of active compound of 1.5% by weight. The solution is then sterilised by filtration, filled into an ampoule and sealed.

### (iv) Injectable Formulation II

A parenteral composition for injection is prepared by dissolving in water a compound of the formula (I) (e.g. in salt form) (2 mg/ml) and mannitol (50 mg/ml), sterile filtering the solution and filling into sealable 1 ml vials or ampoules.

### v) Injectable formulation III

A formulation for i.v. delivery by injection or infusion can be prepared by dissolving the compound of formula (I) (e.g. in a salt form) in water at 20 mg/ml. The vial is then sealed and sterilised by autoclaving.

### vi) Injectable formulation IV

A formulation for i.v. delivery by injection or infusion can be prepared by dissolving the compound of formula (I) (e.g. in a salt form) in water containing a buffer (e.g. 0.2 M acetate pH 4.6) at 20 mg/ml. The vial is then sealed and sterilised by autoclaving.

### (vii) Subcutaneous Injection Formulation

A composition for sub-cutaneous administration is prepared by mixing a compound of the formula (I) with pharmaceutical grade corn oil to give a concentration of 5 mg/ml. The composition is sterilised and filled into a suitable container.

### viii) Lyophilised Formulation

Aliquots of formulated compound of formula (I) are put into 50 ml vials and lyophilized. During lyophilisation, the compositions are frozen using a one-step freezing protocol at (−45° C.). The temperature is raised to −10° C. for annealing, then lowered to freezing at −45° C., followed by primary drying at +25° C. for approximately 3400 minutes, followed by a secondary drying with increased steps if temperature to 50° C. The pressure during primary and secondary drying is set at 80 millitor.

### EQUIVALENTS

The foregoing examples are presented for the purpose of illustrating the invention and should not be construed as imposing any limitation on the scope of the invention. It will readily be apparent that numerous modifications and alterations may be made to the specific embodiments of the invention described above and illustrated in the examples without departing from the principles underlying the invention. All such modifications and alterations are intended to be embraced by this application.

1-78. (canceled)

79. A compound of formula (I): 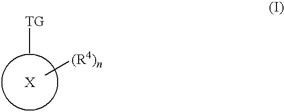
or a salt, solvate, tautomer or N-oxide thereof;
wherein TG is selected from:
(1) a
group: 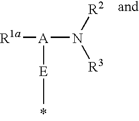
(2) a
group: 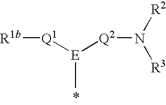
wherein the asterisk (*) represents the point of attachment of the group E to the group X;
A is a saturated hydrocarbon linker group containing from 1 to 7 carbon atoms, the linker group having a maximum chain length of 5 atoms extending between R^{1a} and NR²R³ and a maximum chain length of 4 atoms extending between E and NR²R³, wherein one of the carbon atoms in the linker group may optionally be replaced by an oxygen or nitrogen atom; and wherein the carbon atoms of the linker group A may optionally bear one or more substituents selected from oxo, fluorine and hydroxy, provided that the hydroxy group when present is not located at a carbon atom a with respect to the NR²R³ group and provided that the oxo group when present is located at a carbon atom α with respect to the NR²R³ group;
E is a monocyclic or bicyclic carbocyclic or heterocyclic group;
the group X is a bicyclic heterocyclic group having 8 to 12 ring members of which up to 5 are heteroatoms selected from O, N and S;
R^{1a} is an aryl or heteroaryl group;
R^{1b} is hydrogen or a group R^{1a};
R² and R³ are independently selected from hydrogen, C₁₋₄ hydrocarbyl and C₁₋₄ acyl wherein the hydrocarbyl and acyl moieties are optionally substituted by one or more substituents selected from fluorine, hydroxy, amino, methylamino, dimethylamino and methoxy;
or R² and R³ together with the nitrogen atom to which they are attached form a cyclic group selected from an imidazole group and a saturated monocyclic heterocyclic group having 4-7 ring members and optionally containing a second heteroatom ring member selected from O and N;
or one of R² and R³ together with the nitrogen atom to which they are attached and one or more atoms from the linker group A form a saturated monocyclic heterocyclic group having 4-7 ring members and optionally containing a second heteroatom ring member selected from O and N;
or NR²R³ and the carbon atom of linker group A to which it is attached together form a cyano group;
n is 0 to 4;
each R⁴ is independently selected from oxo; halogen; C₁₋₆ hydrocarbyl optionally substituted by halogen, hydroxy or C₁₋₂ alkoxy; cyano; C₁₋₆ hydrocarbyloxy optionally substituted by halogen, hydroxy or C₁₋₂ alkoxy; CONH₂; CONHR⁹; CF₃; NH₂; NHCOR⁹; NHCONHR⁹; and NHR⁹;
R⁹ is a group R^{9a} or (CH₂)R^{9a}, wherein R^{9a} is a monocyclic or bicyclic group which may be carbocyclic or heterocyclic;
the carbocyclic group or heterocyclic group R^{9a} is optionally substituted by one or more substituents selected from halogen, hydroxy, trifluoromethyl, cyano, nitro, carboxy, amino, mono- or di-C₁₋₄ hydrocarbylamino; a group R^{a}-R^{b} wherein R^{a} is a bond, O, CO, X¹C(X²), C(X²)X¹, X¹C(X²)X¹, S, SO, SO₂, NR^{c}, SO₂NR^{c} or NR^{c}SO₂; and R^{b} is selected from hydrogen, heterocyclic groups having from 3 to 12 ring members, and a C₁₋₈ hydrocarbyl group optionally substituted by one or more substituents selected from hydroxy, oxo, halogen, cyano, nitro, carboxy, amino, mono- or di-C₁₋₄ hydrocarbylamino, carbocyclic and heterocyclic groups having from 3 to 12 ring members and wherein one or more carbon atoms of the C₁₋₈ hydrocarbyl group may optionally be replaced by O, S, SO, SO₂, NR^{c}, X¹C(X²), C(X²)X¹ or X¹C(X²)X¹;
R^{c} is selected from hydrogen and C₁₋₄ hydrocarbyl; and
X¹ is O, S or NR^{c} and X² is ═O, ═S or ═NR^{c};
Q¹ is a bond or a saturated hydrocarbon linker group containing from 1 to 3 carbon atoms, wherein one of the carbon atoms in the linker group may optionally be replaced by an oxygen or nitrogen atom, or an adjacent pair of carbon atoms may be replaced by CONR^{q} or NR^{q}CO where R^{q} is hydrogen, C₁₋₄ alkyl or cyclopropyl, or R^{q} is a C₁₋₄ alkylene chain that links to R^{1b} or to another carbon atom of Q¹ to form a cyclic moiety; and wherein the carbon atoms of the linker group Q¹ may optionally bear one or more substituents selected from fluorine and hydroxy;
Q² is a bond or a saturated hydrocarbon linker group containing from 1 to 3 carbon atoms, wherein one of the carbon atoms in the linker group may optionally be replaced by an oxygen or nitrogen atom; and wherein the carbon atoms of the linker group may optionally bear one or more substituents selected from fluorine and hydroxy, provided that the hydroxy group when present is not located at a carbon atom α with respect to the NR²R³ group; and provided that when E is aryl or heteroaryl, then Q² is other than a bond;
and further provided that the moiety 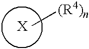
is other than a group (BG1) or (BG2); 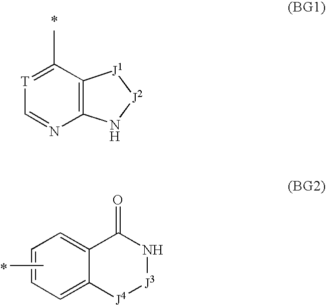
wherein (BG1) and (BG2) are each optionally substituted;
the asterisk (*) represents the point of attachment of the group E;
T is N or a group CR^{z};
J¹-J² represents a group selected from N═C(R^{z}), (R^{z})C═N, (R^{z})N—C(O), (R^{z})₂C—C(O), N═N and (R^{z})C═C(R⁶);
J⁴-J³ is a group N═C(R^{z}) or a group (R^{z})N—CO; and
R^{z} is hydrogen or a substituent.

80. A compound according to claim 79 wherein TG is:
(1) a group:

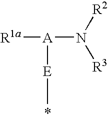

81. A compound according to claim 79 wherein TG is:
(2) a group: 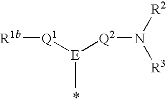
Q¹ is a bond or a saturated hydrocarbon linker group containing from 1 to 3 carbon atoms, wherein one of the carbon atoms in the linker group may optionally be replaced by an oxygen or nitrogen atom, or an adjacent pair of carbon atoms may be replaced by CONR^{q} or NR^{q}CO where R^{q} is hydrogen, C₁₋₄ alkyl or cyclopropyl, or R^{q} is a C₁₋₄ alkylene chain that links to R^{1b} or to another carbon atom of Q¹ to form a cyclic moiety; and wherein the carbon atoms of the linker group Q¹ may optionally bear one or more substituents selected from fluorine and hydroxy.

82. A compound according to claim 81 wherein R^{1b} is a group R^{1a}.

83. A compound according to claim 79 wherein R^{1a} is selected from optionally substituted phenyl, naphthyl, thienyl, furan, pyrimidine and pyridine groups, wherein the optional substituents are selected from hydroxy; C₁₋₄ acyloxy; fluorine; chlorine; bromine; trifluoromethyl; cyano; CONH₂; nitro; C₁₋₄ hydrocarbyloxy and C₁₋₄ hydrocarbyl each optionally substituted by C₁₋₂ alkoxy, carboxy or hydroxy; C₁₋₄ acylamino; benzoylamino; pyrrolidinocarbonyl; piperidinocarbonyl; morpholinocarbonyl; piperazinocarbonyl; five and six membered heteroaryl and heteroaryloxy groups containing one or two heteroatoms selected from N, O and S; phenyl; phenyl-C₁₋₄ alkyl; phenyl-C₁₋₄ alkoxy; heteroaryl-C₁₋₄ alkyl; heteroaryl-C₁₋₄ alkoxy and phenoxy, wherein the heteroaryl, heteroaryloxy, phenyl, phenyl-C₁₋₄ alkyl, phenyl-C₁₋₄ alkoxy, heteroaryl-C₁₋₄ alkyl, heteroaryl-C₁₋₄ alkoxy and phenoxy groups are each optionally substituted with 1, 2 or 3 substituents selected from C₁₋₂ acyloxy, fluorine, chlorine, bromine, trifluoromethyl, cyano, CONH₂, C₁₋₂ hydrocarbyloxy and C₁₋₂ hydrocarbyl each optionally substituted by methoxy or hydroxy.

84. A compound according to claim 79 wherein R² and R³ are independently selected from hydrogen; C₁₋₄ hydrocarbyl and C₁₋₄ acyl wherein the hydrocarbyl and acyl groups are optionally substituted by one or more substituents selected from fluorine, hydroxy, cyano, amino, methylamino, dimethylamino, methoxy and a monocyclic or bicyclic aryl or heteroaryl group.

85. A compound according to claim 79 wherein E is a phenyl ring or a piperazine or piperidine ring.

86. A compound according to claim 79 wherein the moiety 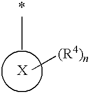
is an optionally substituted bicyclic heteroaryl group, wherein the bicyclic heteroaryl group is a pyridine or pyrimidine ring fused to a 5- or 6-membered carbocyclic or heterocyclic aromatic ring.

87. A compound according to claim 79 wherein the bicyclic group X is selected from:

| | |
|---|---|
| 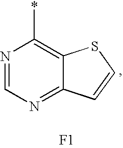 | 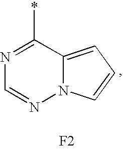 |
| 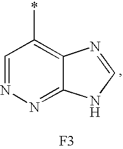 | 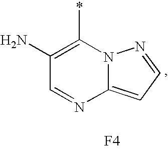 |
| 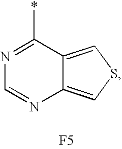 | 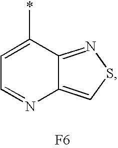 |
| 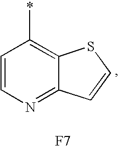 | 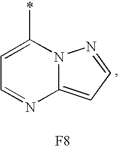 |
| 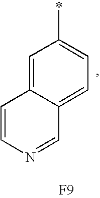 | 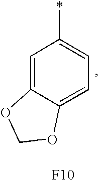 |
| 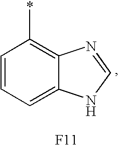 | 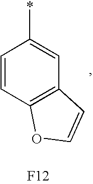 |
| 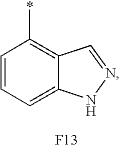 | 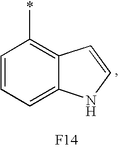 |
| 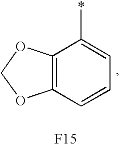 | 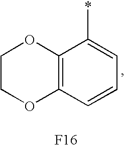 |
| 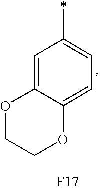 | 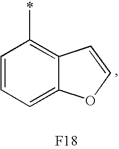 |
| 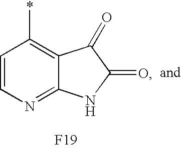 | 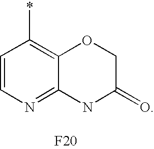 |

88. A compound according to claim 79 having the general formula (II): 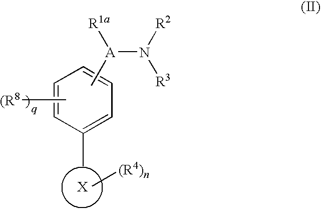
and salts, solvates, tautomers and N-oxides thereof,
wherein the group A is attached to the meta or para position of the benzene ring, q is 0-4; R^{1a}, R², R³ and R⁴ are as defined in claim 79 and R⁸ is selected from hydroxy; halogen; trifluoromethyl; cyano; C₁₋₄ hydrocarbyloxy optionally substituted by C₁₋₂ alkoxy or hydroxy; C₁₋₄ hydrocarbyl optionally substituted by C₁₋₂ alkoxy or hydroxy; and phenyl optionally substituted by halogen, trifluoromethyl, cyano, methyl or methoxy.

89. A compound according to claim 79 having the formula (IIa): 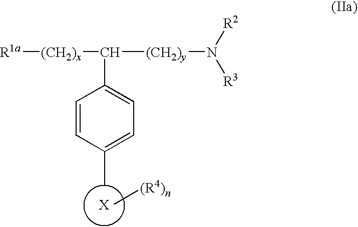
and salts, solvates, tautomers and N-oxides thereof,
wherein X, R^{1a}, R², R³, R⁴ and n are as defined in claim 79, x is 0 or 1 and y is 0, 1 or 2.

90. A compound according to claim 79 having the formula (IIb): 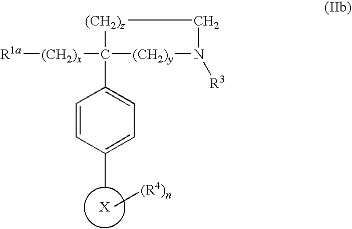
and salts, solvates, tautomers and N-oxides thereof,
wherein X, R^{1a}, R², R³, R⁴, x and y are as defined in claim 79 and z is 0, 1 or 2 provided that the sum of y and z does not exceed 4.

91. A compound according to claim 79 having the general formula (IIc): 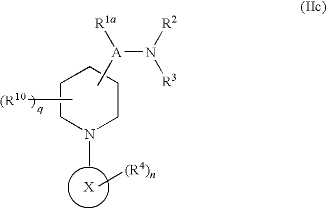
and salts, solvates, tautomers and N-oxides thereof,
wherein the group A is attached to the 3-position or 4-position of the piperidine ring, q is 0-4; X, n, A, R^{1a}, R², R³ and R⁴ are as defined in claim 79 and R¹⁰ is a substituent group selected from halogen, hydroxy, trifluoromethyl, cyano, nitro, carboxy, amino, mono- or di-C₁₋₄ hydrocarbylamino, carbocyclic and heterocyclic groups having from 3 to 12 ring members; a group R^{a}-R^{b} wherein R^{a} is a bond, O, CO, X¹C(X²), C(X²)X¹, X¹C(X²)X¹, S, SO, SO₂, NR^{c}, SO₂NR^{c} or NR^{c}SO₂; and R^{b} is selected from hydrogen, carbocyclic and heterocyclic groups having from 3 to 12 ring members, and a C₁₋₈ hydrocarbyl group optionally substituted by one or more substituents selected from hydroxy, oxo, halogen, cyano, nitro, carboxy, amino, mono- or di-C₁₋₄ hydrocarbylamino, carbocyclic and heterocyclic groups having from 3 to 12 ring members and wherein one or more carbon atoms of the C₁₋₈ hydrocarbyl group may optionally be replaced by O, S, SO, SO₂, NR^{c}, X¹C(X²), C(X²)X¹ or X¹C(X²)X;
R^{c} is selected from hydrogen and C₁₋₄ hydrocarbyl; and
X¹ is O, S or NR^{c} and X² is ═O, ═S or ═NR^{c}.

92. A compound according to claim 79 having the general formula (III): 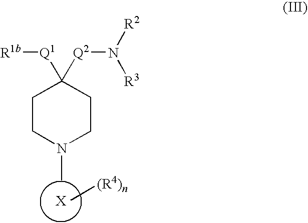
and salts, solvates, tautomers and N-oxides thereof,
wherein X, R^{1b}, R⁴, Q¹, Q² and NR²R³ are as defined in claim 79.

93. A compound according to claim 79 having the formula (IV): 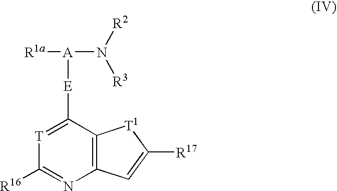
and salts, solvates, tautomers and N-oxides thereof,
wherein T is CH or N, T¹ is S, O or NR¹⁸; R¹⁶ is hydrogen or a group R¹⁰, R¹⁷ is hydrogen or a group R⁴; R¹⁸ is hydrogen or C₁₋₄ alkyl; and A, E, R^{1a} to R⁴ and R¹⁰ are as defined in claim 79.

94. A compound according to claim 92 having the general formula (VI): 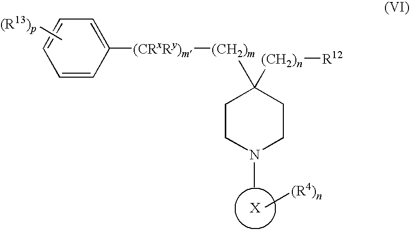
and salts, solvates, tautomers and N-oxides thereof,
wherein m is 0, 1 or 2; m′ is 0 or 1 provided that the sum of m and m′ is in the range 0 to 2; n is 0 or 1; p is 0, 1, 2 or 3; R^{x} and R^{y} are the same or different and each is selected from hydrogen, methyl and fluorine; R¹² is CN or NR²R³ and each R¹³ is independently selected from R¹⁰ wherein X, R², R³, R⁴ and R¹⁰ are as defined in claim 92.

95. A compound according to claim 94 having the formula (VII): 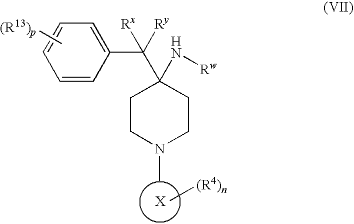
and salts, solvates, tautomers and N-oxides thereof,
wherein R^{x}, R^{y} and R^{w} are each independently hydrogen or methyl, and X, n, p, R⁴ and R¹³ are as defined in claim 94.

96. A compound according to claim 92 having the formula (VIII) 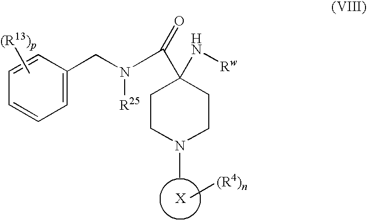
and salts, solvates, tautomers and N-oxides thereof,
wherein R²⁵ is hydrogen or methyl and X, R¹³, R⁴ and R^{w} are as defined in claim 92.

97. A method for prophylaxis or treatment of a disease state or condition mediated by protein kinase A or protein kinase B, which method comprises administering to a subject in need thereof a compound as defined in claim 79 or a salt, solvate, tautomer or N-oxide thereof.

98. A pharmaceutical composition comprising a compound as defined in claim 79 or a salt, solvate, tautomer or N-oxide thereof and a pharmaceutically acceptable carrier.
